(19) United States
(12) Patent Application Publication
(10) Pub. No.: US 2026/0242354 A1
(43) Pub. Date: Aug. 20, 2026

(54) TARGETED THERANOSTIC AGENTS FOR IMAGING AND TREATING CANCER

(71) Applicant: THE BOARD OF REGENTS OF THE UNIVERSITY OF TEXAS SYSTEM, Austin, TX (US)

(72) Inventors: Xiankai Sun, Dallas, TX (US); Guiyang Hao, Dallas, TX (US); Sashi Debnath, Dallas, TX (US)

(21) Appl. No.: 19/149,650

(22) PCT Filed: Jan. 22, 2024
(86) PCT No.: PCT/US2024/012432

§ 371 Date: Jul. 21, 2025

Related U.S. Application Data
(60) Provisional application No. 63/440,234, filed on Jan. 20, 2023.

(51) Int. Cl. C07D 401/14; A61K 51/04

(52) U.S. Cl. CPC C07D 401/14; A61K 51/0497; A61K 2123/00

(57) ABSTRACT

The present invention provides SV2A-targeted theranostic agents for use in imaging and treatment of cancer innervation or neuroendocrine differentiation of cancer.

### CROSS REFERENCE TO RELATED APPLICATION

This application claims priority to U.S. Provisional Patent Application Ser. No. 63/440,234 filed Jan. 20, 2023 and titled “TARGETED THERANOSTIC AGENTS FOR IMAGING AND TREATING CANCER,” which is incorporated by reference herein in its entirety.

### BACKGROUND

### 1. Field

The present disclosure generally relates to SV2A-targeted theranostic agents for use in imaging and treatment of cancer innervation or neuroendocrine differentiation of cancer.

### 2. Discussion of Related Art

Neuroendocrine differentiation (NED) in cancers is defined in general as the presence of neurosecretory granules in neoplastic cells, resembling synaptic vesicles. Histologically, they are characterized by structural patterns and cytological features reminiscent of nonneoplastic neuroendocrine (NE) cells and expression of NE markers. The presence of NED has been recognized in many cancer types, not only the commonly seen neuroendocrine tumors but also prostate cancer, breast cancer, colorectal cancer, etc. In addition, NED has been shown in association with distal metastases and other unfavorable features contributing to poor clinical outcomes. For example, although the 5-year cancer-specific survival rate for patients with localized and regional prostate cancer is nearly 100%, the 5-year cancer-specific survival sharply drops to 31% for metastatic castration-resistant prostate cancer (mCRPC). Classified as neuroendocrine PC (NEPC), a subset of mCRPC with universal neuroendocrine differentiation (NED) features manifests even worse prognosis with overall survival of <1 year. The current therapeutic regimen for NEPC is limited to cisplatin or carboplatin in combination with taxanes but showing minimal effects. The predominant type of PCa (prostate cancer), adenocarcinoma PCa (AdPC), has been documented with focal NED features (10% to 100%). A positive correlation has thus been suggested between NED and the poor prognosis in PCa, indicating that NED can potentially serve as a prognostic indicator for PCa.

In addition, NED has been implicated in neuron-cancer interactions or innervations during cancer development and progression. For example, both epithelial and prostate cancer cells can undergo perineural invasion and adopt a true neural-mimicking phenotype, by which prostate cancer cells circumvent the stressful situations resulting from androgen deprivation therapy (ADT). NE-like cells produce and secrete a cocktail of mediators commonly seen in the nervous system, and these neuropeptides have mitogenic effects that endure the growth and survival of adjacent cancer cells.

Current diagnosis of NED relies on the pathologic assessment of biopsies, but its accuracy is limited by sampling bias and the fact that repeated biopsy procedures are not clinically feasible. In addition, there is always a lag between the underlying molecular initiation and the phenotypic appearance. Given its non-invasive and quantitative features coupled with inherent superior sensitivity, positron emission tomography (PET) has become a molecular imaging tool revolutionizing cancer management. For example, the US FDA approved [⁶⁸Ga]Ga-DOTATATE (NETSPOT™) for PET imaging of somatostatin receptor 2 (SSTR2) positive neuroendocrine tumors (NETs), Choline C-11, and Fluciclovine F-18 (Axumin®) for PET imaging diagnosis of recurrent prostate cancer, and [⁶⁸Ga]Ga-PSMA-11 and [¹⁸F]F-DCFPyL for detection of prostate-specific membrane antigen (PSMA)-positive lesions in men with prostate cancer. However, none of the agents is of potential use to detect NED in cancer as those imaging targets are either independent (SSTR2) or inverted (choline metabolism, decreased proliferation rate, and reduced PSMA expression) with NE progression. Noninvasive assessment of NED in NE-featured cancer types is an unmet clinical need.

In search for a target to develop NED-targeted theranostics, it has been found that synaptic vesicle glycoprotein 2 isoform A (SV2A) can serve as a promising candidate. Currently, NED is characterized pathologically by chromogranin A (CgA) and synaptophysin (SYP) staining with the former being more specific and the latter being more sensitive. SYP represents a family of proteins present in synaptic vesicles that store and release classic neurotransmitters in both the nervous system and neuroendocrine tumors (NETs). An upregulated expression of SYP reflects activated synaptic machinery that may cause or enhance tumor innervation and growth. It has been reported that SV2A can be used for pathological assessment of NED in NETs, interestingly, with greater similarity to SYP than to CgA. In addition, SV2A has been identified as the key membrane receptor for botulinum neurotoxin, which might be of therapeutic potential for the treatment of tumors featuring NE phenotypes, including CRPC. Furthermore, the expression of SV2A was also found in correlation with the ability of colorectal cancer stem cells to produce functional neurons, indicative of its role in cancer innervation. Among the three synaptic vesicle glycoprotein isoforms (SV2A, SV2B, and SV2C), SV2A is the most dominant one found in cancers. Unlike CgA present in both blood and tumors, SV2A and SYP are confined to innervated tumors, which is a desired feature for oncological imaging.

SV2A-targeted conjugates with high tumor contrast, low brain and other background tissue uptake, and efficient renal clearance are desired for imaging and therapy of cancer innervation and NED. Several SV2A-targeting molecules have been reported in recent years. Among them, [¹¹C]UCB-J and [¹⁸F]UCB-J have proven to be excellent PET probes to image and quantify synaptic density in the brain and both have been applied to the studies of a variety of neurodegenerative and neuropsychiatric disorders. Previously multivalent strategy showed improvement in tumor accumulation for a tumor targeting conjugates.

What is needed are SV2A-targeted theranostic agents for imaging and treatment of cancer innervation or neuroendocrine differentiation of cancer.

### FIGURES

FIG. 1A is a general schematic representation of the multistep organic synthesis of CB-TE2A-(PEG₃-AMTP) and CB-TE2A-(PEG₃-AMTP)₂.

FIG. 1B is a schematic representation of the multistep organic synthesis of CB-TE2A-(PEG₃-AMTP).

FIG. 1C is a schematic representation of the multistep organic synthesis of CB-TE2A(^{t}Bu)₂-(PEG₃-AMTP)₂.

FIG. 2 depicts the characterization of (4R)-1-[(3-bromo-4-pyridyl)methyl]-4-(3,4,5-trifluorophenyl)pyrrolidin-2-one C by LC-MS.

FIG. 3 depicts the characterization of (4R)-1-[(3-bromo-4-pyridyl)methyl]-4-(3,4,5-trifluorophenyl)pyrrolidin-2-one C by ¹H NMR.

FIG. 4 depicts the characterization AMTP-PEG₃-NH₂, compound E by LC-MS.

FIG. 5 depicts the characterization AMTP-PEG₃-NH₂, compound E by ¹H NMR.

FIG. 6 depicts the characterization AMTP-PEG₃-NH₂, compound E by ¹³C NMR.

FIG. 7 depicts the characterization CB-TE2A(^{t}Bu)₂-PEG₃-AMTP by LC-MS.

FIG. 8 depicts the characterization CB-TE2A-PEG₃-AMTP by LC-MS.

FIG. 9 depicts the characterization CB-TE2A(^{t}Bu)₂-(PEG₃-AMTP)₂ by LC-MS.

FIG. 10 depicts the characterization CB-TE2A-(PEG₃-AMTP)₂ by LC-MS.

FIG. 11 is a schematic representation of the multistep organic synthesis of CB-TE2A-(PEG₃-AMTP)₂ and ^{nat}Cu-CB-TE2A-(PEG₃-AMTP)₂.

FIG. 12 depicts the characterization ^{nat}Cu-CB-TE2A-PEG₃-(AMTP)₂ by LC-MS.

FIG. 13 depicts the characterization [⁶⁴Cu]Cu-CB-TE2A-PEG₃-AMTP by HPLC.

FIG. 14 depicts the characterization [⁶⁴Cu]Cu-CB-TE2A-(PEG₃-AMTP)₂ by HPLC.

FIGS. 15A-F depict SV2A protein expression analyses in different cell lines with (β-actin as a loading control.

FIG. 15A is a graphical representation of Western blot of different neuroendocrine cancer cell lines with SV2A protein expression.

FIG. 15B is a graphical representation of Western blot of SV2A in prostate cancer (LNCaP, 22RV1, PC3, DU145, and NCI-H660) cells.

FIG. 15C is a graphical representation of Western blot of DU145 prostate cancer cell lines showing elevated expression of SV2A protein.

FIG. 15D is a graphical representation of Western blot of PC3 prostate cancer cell lines showing elevated expression of SV2A protein.

FIG. 15E is a graphical representation of Western blot of LNCaP prostate cancer cell lines showing elevated expression of SV2A protein.

FIG. 15F is a graphical representation of Western blot of IIG5 prostate cancer cell lines showing elevated expression of SV2A protein.

FIGS. 16A & 16B is a graphical representation cell uptake of [⁶⁴Cu]Cu-CB-TE2A-PEG₃-AMTP (16A) and [⁶⁴Cu]Cu-CB-TE2A-(PEG₃-AMTP)₂ (16B) in the absence and presence of blocker SynVesT-1 in PC3-VC (SV2A^{high}) and LnCap (SV2A^{low}) cells.

FIGS. 16C & 16D show normalized SV2A selective cell uptake of [⁶⁴Cu]Cu-CB-TE2A-PEG₃-AMTP (16C) and [⁶⁴Cu]Cu-CB-TE2A-(PEG₃-AMTP)₂ (16D) in PC3-VC (SV2A^{high}) and LnCap (SV2A^{low}) cells (uptake in SV2A^{low} cells was set at 1).

FIGS. 16E & 16F show internalization of [⁶⁴Cu]Cu-CB-TE2A-PEG₃-AMTP (16E) and [⁶⁴Cu]Cu-CB-TE2A-(PEG₃-AMTP)₂ (16F) in PC3-VC (SV2A^{high}). Data presented as average counts±s.d. (n=3). * p=0.0520, ** p=0.0285, * p=0.0006, *** p=0.0007, #p=0.0285, ##p=0.0007 (unpaired t-tests performed by GraphPad Prizm 9.5.1.733).

FIG. 17A shows PET/CT images for [⁶⁴Cu]Cu-CB-TE2A-PEG₃-AMTP in SCID mice bearing SV2A^{high} H720 tumor.

FIG. 17B shows PET/CT images for [⁶⁴Cu]Cu-CB-TE2A-PEG₃-AMTP in SCID mice bearing SV2A^{high} BON1 tumor.

FIG. 17C shows time-activity curves (TAC) of [⁶⁴Cu]Cu-CB-TE2A-PEG₃-AMTP in SV2A^{high} H720 tumor mice.

FIG. 17D shows time-activity curves (TAC) of [⁶⁴Cu]Cu-CB-TE2A-PEG₃-AMTP in SV2A^{high} BON1 tumor mice.

FIGS. 18A & 18B show time-activity curves (TAC) of [⁶⁴Cu]Cu-CB-TE2A-PEG₃-AMTP in SV2A^{high} H720 tumor mice organs (% ID/g, n=3).

FIGS. 19A & 19B show time-activity curves (TAC) of [⁶⁴Cu]Cu-CB-TE2A-PEG₃-AMTP in SV2A^{high} BON1 tumor mice organs (% ID/g, n=3).

FIG. 20A shows PET/CT images for [⁶⁴Cu]Cu-CB-TE2A-(PEG₃-AMTP)₂ in SCID mice bearing SV2A^{high} H720 tumor.

FIG. 20B shows PET/CT images for [⁶⁴Cu]Cu-CB-TE2A-(PEG₃-AMTP)₂ in SCID mice bearing SV2A^{high} DU145-VC, SV2A^{low} DU145_sgPTP1B dual tumors and SV2A^{high} PC3-VC, SV2A^{low} PC3_sgPTP1B dual tumors.

FIG. 20C shows TAC of [⁶⁴Cu]Cu-CB-TE2A-(PEG₃-AMTP)₂ in SV2A^{high} H720 tumors and muscle (% ID/g, n=4).

FIG. 20D shows TAC of [⁶⁴Cu]Cu-CB-TE2A-(PEG₃-AMTP)₂ (n=4) and [⁶⁴Cu]Cu-CB-TE2A-PEG₃-AMTP (n=4) in SV2A^{high} H720 tumors (% ID/g).

FIG. 20E shows TAC of [⁶⁴Cu]Cu-CB-TE2A-(PEG₃-AMTP)₂ in SV2A^{high} DU145-VC, SV2A^{low} DU145_sgPTP1B tumors and muscle (% ID/g, n=4).

FIG. 20F shows TAC of [⁶⁴Cu]Cu-CB-TE2A-(PEG₃-AMTP)₂ in SV2A^{high} PC3-VC, SV2A^{low} PC3_sgPTP1B tumors and muscle (% ID/g).

FIG. 21A shows PET/CT images for [⁶⁴Cu]Cu-CB-TE2A-(PEG₃-AMTP)₂ in SCID mice bearing SV2A^{high} H720 tumor.

FIG. 21B shows time-activity curves (TAC) of [⁶⁴Cu]Cu-CB-TE2A-(PEG₃-AMTP)₂ in SV2A^{high} H720 tumors and muscle (% ID/g, n=3).

FIG. 21C shows time-activity curves (TAC) of [⁶⁴Cu]Cu-CB-TE2A-(PEG₃-AMTP)₂ in SV2A^{high} H720 (n=4) and H727 (n=3) tumors (% ID/g).

FIGS. 22A-22B show time-activity curves (TAC) of [⁶⁴Cu]Cu-CB-TE2A-(PEG₃-AMTP)₂ in SV2A^{high} H720 tumor mice organs (% ID/g, n=4).

FIGS. 23A-23B show time-activity curves (TAC) of [⁶⁴Cu]Cu-CB-TE2A-(PEG₃-AMTP)₂ in SV2A^{high} DU145-control, SV2A^{low} DU145_sgPTP1B tumor mice organs (% ID/g, n=4).

FIGS. 24A-24B show time-activity curves (TAC) of [⁶⁴Cu]Cu-CB-TE2A-(PEG₃-AMTP)₂ in SV2A^{high} PC3-control, SV2A^{low} PC3_sgPTP1B tumor mice organs (% ID/g, n=4).

FIGS. 25A-25E show the immunostaining of SV2A in NET tumors. The red bar in each image represents scale of 100 μm (upper panel) and 200 μm (lower panel).

FIG. 26 is a graphical representation of the multistep organic synthesis of DOTA-PEG₃-AMTP and ^{nat}Ga-DOTA-PEG₃-AMTP.

FIGS. 27A-27B depict the characterization of DOTA-PEG₃-AMTP and ^{nat}Ga-DOTA-PEG₃-AMTP by LC-MS.

FIG. 28 depicts the radiochemical purity of [⁶⁸Ga]Ga-DOTA-PEG₃-AMTP by radio-HPLC to determine the radiochemical purity (>99%).

FIG. 29 depicts the PET/CT images for SV2A⁺ NCI-H660 tumors were observable at 10-20 min time duration using [⁶⁸Ga]Ga-DOTA-PEG₃-AMTP.

FIG. 30 is a graphical representation showing the quantitative uptake analysis of [⁶⁸Ga]Ga-DOTA-PEG₃-AMTP.

### SUMMARY OF THE INVENTION

One aspect of the present disclosure encompasses the compound of Formula (I):

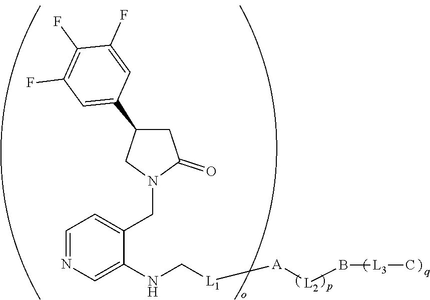

wherein:
L₁, L₂, and L₃ are linkers and are independently selected from a group consisting of at least one peptide linker, at least one polyethylene glycol (PEG) linker, a disulfide linker, an albumin binding entity, absent, and a combination thereof,
A is a metal chelator or a chemotherapeutic agent,
B is a heterocyclic ring, a polyamidoamine dendrimer, a peptide, a disulfide, or absent, C is a chemotherapeutic agent,

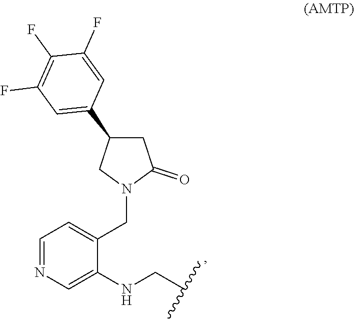 [SMILES: *CNc1cnccc1CN1C[C@@H](c2cc(F)c(F)c(F)c2)CC1=O]

or absent,
o is an integer from 0 to 6,
p is an integer from 0 or 1,
q is an integer from 1 to 6, and
wherein when o is 0, C is AMTP.

Another aspect of the present disclosure encompasses a pharmaceutical composition comprising a composition of the compound of Formula (I) and at least one pharmaceutically acceptable excipient or carrier.

In still another aspect of the present disclosure encompasses a method of treating and/or detecting a cancer, the method comprising administering an effective amount of a composition of the compound of Formula (I) or a pharmaceutical composition comprising a composition of the compound of Formula (I) and at least one pharmaceutically acceptable excipient or carrier to a subject in need thereof, wherein the subject in need thereof has or is suspected of one or more cancers.

In yet another aspect of the present disclosure encompasses a method of imaging at least one region of a subject's body, the method comprising: administering to the subject at least one composition compound of Formula (I) or a pharmaceutical composition comprising a composition of the compound of Formula (I) and at least one pharmaceutically acceptable excipient or carrier; and detecting the at least one compound by nuclear medicine imaging in at least one region of the body of the subject; thereby generating an image of the at least one region of the body of the subject.

In still another aspect of the present disclosure encompasses a method of monitoring and/or evaluating the effectiveness of treatment or therapy for cancer in a subject's body, the method comprising: administering to the subject compound of Formula (I) or a pharmaceutical composition comprising a composition of the compound of Formula (I) and at least one pharmaceutically acceptable excipient or carrier; detecting the at least one compound by PET, SPECT, CT, MRI, or a combination thereof in at least one region of the body of the subject;

Other features and iterations of the invention are described in more detail below.

### DETAILED DESCRIPTION OF THE INVENTION

The present disclosure provides the compounds of Formula (I), pharmaceutical compositions comprising the compound of Formula (I) and at least one pharmaceutical excipient or carrier; methods of treating and/or detecting a cancer; methods of imaging at least one region in a subject's body; and a method of monitoring and/or evaluating effectiveness of treatment or therapy for a cancer in a subject's body.

### I. Compounds of Formula (I)

An aspect of the present disclose encompasses compound of:

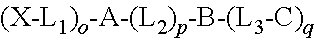

wherein X is

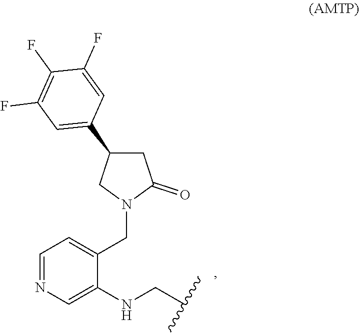 [SMILES: *CNc1cnccc1CN1C[C@@H](c2cc(F)c(F)c(F)c2)CC1=O]

a conformational isomer of AMTP, or an analog of AMTP,
L₁, L₂, and L₃ are linkers and L₁, L₂, and L₃ are independently selected from a group consisting of at least one peptide linker, at least one polyethylene glycol (PEG) linker, a disulfide linker, an albumin binding entity, absent, and a combination thereof,
A is a metal chelator or a chemotherapeutic agent,
B is a heterocyclic ring, a polyamidoamine dendrimer, a peptide, a disulfide, or absent,
C is a chemotherapeutic agent, (AMTP), or absent,
o is an integer from 0 to 6,
p is 0 or 1,
q is an integer from 1 to 6, and
wherein when o is 0, C is AMTP or an analog of AMTP.

The AMTP used in this disclosure may be AMTP, or an analog of AMTP. For example, suitable analogs may include the 2,3,5-trifluoro substituted phenyl ring. Other analogs of the AMTP may have one or two fluorine atoms or an alkyl group on the phenyl ring, or an alkyl group or a fluorine atom on the 2-position of the pyridine ring. These AMTP or analogs of AMTP are known to act as ligands to target the SV2A protein.

One aspect of the present disclosure encompasses the compounds of Formula (I) or Formula (Ia):

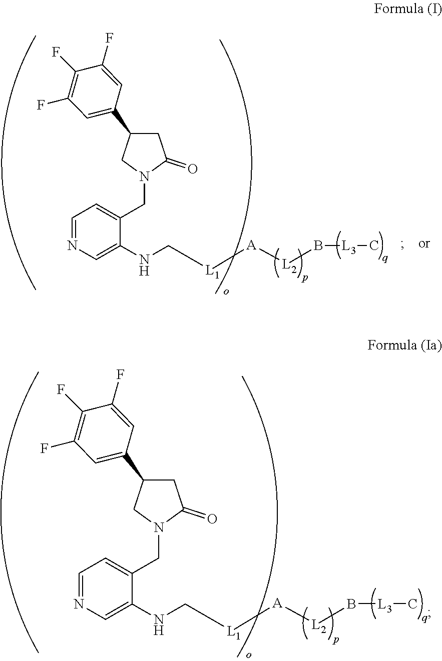

wherein:
x is a halogen;
L₁, L₂, and L₃ are linkers and are independently selected from a group consisting of at least one peptide linker, at least one polyethylene glycol (PEG) linker, a disulfide linker, an albumin binding entity, absent, and a combination thereof;
A is a metal chelator or a chemotherapeutic agent,
B is a heterocyclic ring, a polyamidoamine dendrimer, a peptide, a disulfide, or absent,
C is a chemotherapeutic agent,

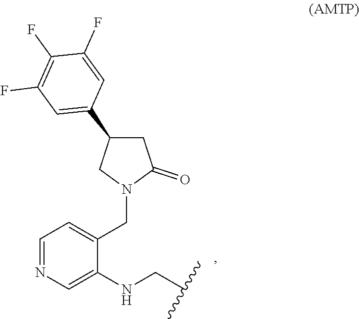 [SMILES: *CNc1cnccc1CN1C[C@@H](c2cc(F)c(F)c(F)c2)CC1=O]

or absent,
o is an integer from 0 to 6,
p is 0 or 1,
q is an integer from 1 to 6, and
wherein when o is 0, C is AMTP.

In some embodiments, L₁, L₂, and L₃ are independently selected from a group consisting of at least one peptide linker, at least one polyethylene glycol (PEG) linker, a disulfide linker, an albumin binding entity, absent, and a combination thereof.

In other embodiments, L₁, L₂, and L₃ are independently selected from a group consisting of at least one peptide linker comprising about 2 to about 10 amino acid residues, a polyethylene glycol linker comprising from 1 to 12 ethylene glycol repeating units, a disulfide linker, an albumin binding entity, absent, and a combination thereof.

In some embodiments, L₁, is a polyethylene glycol link comprising from 1 to 12 ethylene glycol repeating units, and L₂, and L₃ are absent; L₁ is absent, L₂ is a peptide linker comprising about 2 to about 10 amino acid residues wherein one of the amino acid residues is lysine, and L₃ is a polyethylene glycol linker comprising from 1 to 12 ethylene glycol repeating units; L₁ is absent; L₂ and L₃ are polyethylene glycol linkers comprising from 1 to 12 ethylene glycol repeating units; L₁ is absent, L₂ is a peptide linker comprising 2 to about 10 amino acid residues wherein one of the amino acid residues is lysine and an albumin binding entity and L₃ is a peptide linker comprising 2 to about 10 amino acid residues wherein one of the amino acid residues is lysine; L₁ is an albumin binding entity and an polyethylene glycol linker comprising from 1 to 12 ethylene glycol repeating units and L₂ and L₃ are absent; L₁, L₂, and L₃ are polyethylene glycol linkers comprising from 1 to 12 ethylene glycol repeating units; L₁ is a polyethylene glycol linker comprising from 1 to 12 ethylene glycol repeating unit and a peptide and L₂ and L₃ are absent; L₁ is a polyethylene glycol linker comprising from 1 to 12 ethylene glycol repeating units and a peptide and L₂ and L₃ are absent; L₁ is a polyethylene glycol linker comprising from 1 to 12 ethylene glycol repeating units, L₂ is a polyethylene glycol linker comprising from 1 to 12 ethylene glycol repeating units, and L₃ is a disulfide linker; L₁ is absent, L₂ is a polyethylene glycol linker comprising from 1 to 12 ethylene glycol repeating units and a disulfide group, and L₃ is a polyethylene glycol linker comprising from 1 to 12 ethylene glycol repeating units; or L₁ is absent, L₂ is a disulfide group and a peptide linker comprising 2 to about 10 amino acid residues wherein one of the amino acid residues is lysine or a peptide linker comprising 2 to about 10 amino acid residues wherein one of the amino acid residues is lysine, an albumin binding entity, and a disulfide group, and L₃ is a peptide linker comprising 2 to about 10 amino acid residues wherein one of the amino acid residues is lysine.

In some embodiments, A is a metal chelator. In some embodiments, A is a metal chelator selected from a group consisting of 1,4,7-triazacyclononane-1,4,7-triacetic acid (NOTA), 5-(8-methyl-3,6,10,13,16,19-hexaaza-bicyclo[6.6.6]icosan-1-ylamino)-5-oxopentanoic acid (MeCOSar), 1,4,7,10-tetraazacyclododecane-1,4,7,10-tetraacetic acid (DOTA), 4,11-bis(carboxymethyl)-1,4,8,11tetraazabicyclo[6.6.2]hexadecane (CB-TE2A), and N,N′-bis-[2-hydroxy-5-(carboxyethyl)benzyl]ethylenediamine-N, N′-diacetic acid (HBED-CC. In some embodiments, A is a selected from the group consisting of MeCOSar, DOTA, CB-TE2A, NOTA, and HBED-CC.

In general, the metal chelator is incorporated to label the compounds with one radionuclide. Non-limiting examples of suitable radionuclides may be ⁶⁷Ga, ¹¹¹In, ^{99m}Tc, ¹³¹I, ¹²³I, ¹²⁵I, ¹⁰³Pd, ⁶⁸Ga, ¹⁸F, ⁶⁰Cu, ⁶¹Cu, ⁶²Cu, ⁶⁴Cu, ⁹⁰Y, ⁶⁷Cu, ¹⁸⁶Re, ¹⁸⁸Re, ¹⁹⁸Au, ¹⁵³Sm, ¹⁷⁷Lu, ²¹³Bi, ²¹²Pb, ²²³Ra, ²¹¹At, or ²²⁵Ac. In some embodiments, the radionuclide may be ⁶⁷Ga, or ⁶⁴Cu.

In some embodiments, A is a chemotherapeutic agent. In other embodiments, the chemotherapeutic agent is a highly potent chemotherapeutic drug (chemo). In general, the highly potent chem drug molecule may be an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof. Non-limiting examples of suitable highly potent chem drug include but are not limited to benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lipoplatin, lomustine (CCNU), mafosfamide, mannosulfan, mechlorethamine, mechlorethamine oxide hydrochloride, melphalan, meturedopa, mustine (mechlorethamine), mitobronitol, nimustine, novembichin, oxaliplatin, phenesterine, piposulfan, prednimustine, ranimustine, satraplatin, semustine, temozolomide, thiotepa, treosulfan, triaziquone, triethylenemelamine, triethylenephosphoramide (TEPA), triethylenethiophosphaoramide (thiotepa), trimethylolomelamine, trofosfamide, uracil mustard and uredopa, aminopterin, ancitabine, azacitidine, 8-azaguanine, 6-azauridine, capecitabine, carmofur (1-hexylcarbomoyl-5-fluorouracil), cladribine, clofarabine, cytarabine (cytosine arabinoside (Ara-C)), decitabine, denopterin, dideoxyuridine, doxifluridine, enocitabine, floxuridine, fludarabine, 5-fluorouracil, gemcetabine, hydroxyurea (hydroxycarbamide), leucovorin (folinic acid), 6-mercaptopurine, methotrexate, nafoxidine, nelarabine, oblimersen, pemetrexed, pteropterin, raltitrexed, tegofur, tiazofurin, thiamiprine, tioguanine (thioguanine), and trimetrexate, aclacinomysin, aclarubicin, actinomycins, adriamycin, aurostatin (for example, monomethyl auristatin E), authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, epoxomicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, sparsomycin, streptonigrin, streptozocin, tubercidin, valrubicin, ubenimex, zinostatin, and zorubicin, cabazitaxel, colchicines, demecolcine, docetaxel, epothilones, ixabepilone, macromycin, omacetaxine mepesuccinate, ortataxel, paclitaxel (for example, DHA-paclitaxel), taxane, tesetaxel, vinblastine, vincristine, vindesine, and vinorelbine, amsacrine, etoposide (VP-16), irinotecan, mitoxantrone, RFS 2000, teniposide, topotecan, aminoglutethimide, antiestrogen, aromatase inhibiting 4(5)-imidazoles, bicalutamide, finasteride, flutamide, fluvestrant, goserelin, 4-hydroxytamoxifen, keoxifene, leuprolide, LY117018, mitotane, nilutamide, onapristone, raloxifene, tamoxifen, toremifene, trilostane, alemtuzumab, cartumaxomab, edrecolomab, epratuzumab, gemtuzumab, gemtuzumab ozogamicin, glembatumumab vedotin, ibritumomab tiuxetan, reditux, rituximab, tositumomab, and trastuzumab; protein kinase inhibitors such as bevacizumab, cetuximab, crizonib, dasatinib, erlotinib, gefitinib, imatinib, lapatinib, mubritinib, nilotinib, panitumumab, pazopanib, sorafenib, sunitinib, toceranib, vandetanib; angiostatin, bevacizumab, denileukin diftitox, endostatin, everolimus, genistein, interferon alpha, interleukin-2, interleukin-12, pazopanib, pegaptanib, ranibizumab, rapamycin (sirolimus), temsirolimus, thalidomide, bortazomib, erythropoietin, interleukins (e.g., IL-1, IL-2, IL-3, IL-6), leukemia inhibitory factor, interferons, romidepsin, thrombopoietin, TNF-α, CD30 ligand, 4-1BB ligand, Apo-1 ligand, aminolevulinic acid, methyl aminolevulinate, retinoids (alitretinon, tamibarotene, tretinoin), temoporfin, anagrelide, arsenic trioxide, asparaginase, bexarotene, bropirimine, celecoxib, chemically linked Fab, efaproxiral, etoglucid, ferruginol, lonidamide, masoprocol, miltefosine, mitoguazone, talapanel, trabectedin, vorinostat, vinblastine, cyclophosphamide/cytophosphane, laulimalide, mytansine, methotrexate, mitomycin C, bleomycin, dacarbazine, etoposide, epirubicin, capecitabine, daunorubicin/daunomycin, carboquone, carmustine, chlorambucil, chlorzoxazone, ifosfamide, gleostine/lomustine/semustine, mafosfamide, melphalan, nimustine, prednimustine, ranimustine, temozolomide, uramustine/uracil mustard, aminopterin, azacitidine, carmofur (1-hexylcarbomoyl-5-fluorouracil), cladribin, clofarabine, decitabine, dideoxyuridine, floxuridine, fludarabine, gemcetabine, leucovorin (folinic acid), nelarabine, pemetrexed, tioguanine, doxorubicin/adriamycin, monomethyl auristatin E, dactinomycin, idarubicin, cabazitaxel, docetaxel, ixabepilone, omacetaxine mepesuccinate, paclitaxel, mitoxantrone, topotecan, laulimalide, maitansine/maytansine, fingolimod, and mertansine. In one embodiment, the highly potent chem drug is Fingolimod (FTY720). In another embodiment, the highly potent chem drug is mertansine (DM1).

In some embodiments, B is a heterocyclic ring, a polyamidoamine dendrimer, a peptide, a disulfide, or absent. In other embodiments, B is a heterocyclic ring selected from a group consisting of triazine, a pyrimidine, a piperazine, a pyrazine, an imidazole, a pyrazole, or a pyridine; an albumin binding entity; a polyamidoamine dendrimer (PAMAM dendrimer) selected from a group consisting of a generation 0 (G0) polyamidoamine dendrimer, a generation 1 (G1) polyamidoamine dendrimer, or a generation 2 (G2) polyamidoamine dendrimer; a peptide; a disulfide; or absent. In some embodiments, B is absent, a peptide, or a triazine.

In some embodiments, C is a chemotherapeutic agent, AMTP, or absent. In other embodiments, C is a highly potent chem drug molecules as described above; AMTP; or absent. In some embodiments, C is absent, AMTP, DM1, or FTY720.

In some embodiments, the AMTP is AMTP, or an analog of AMTP. Analogs of AMTP include, but are not limited to, those disclosed in Mercier, J; et al., ChemMedChem 2014, 9, 693 and Mercier, J; Provins, L; Valade, A; Drug Discovery Today: Technologies, Imaging Technologies in Drug Discovery, Volume 25, No. 2017, 45, the disclosures of which are hereby incorporated by reference in their entirety.

In some embodiments, o is an integer from 0 to 6; p is an integer from 0 or 1; and q is an integer from 1 to 6 wherein when o is 0, C is AMTP. In some embodiments, o is 3; p is 0; and q is 0; o is 0; p is 1; and m is 2, o is 0; p is 1; and q is 2; o is 0; p is 1; and q is an integer from 1 to 6; o is 1; p is 0; and q is 0; o is 2; p is 1; and q is 2; o is 1; p is 0; and q is 0; o is 2; p is 0; and q is 0; o is 1; p is 0; and q is 0; o is 1; p is 0; and q is 0; o is 2; p is 1; and q is 1; o is 2; p is 1; and q is 2; and o is 0; p is 1; q is an integer from 1 to 6.

In an embodiment, A is NOTA; B is absent; C is absent; L₁ is a polyethylene glycol linker comprising from 1 to 12 ethylene glycol repeating units; L₂ and L₃ are absent; o is 3; p is 0; and q is 0; as shown in the compound of Formula (II):

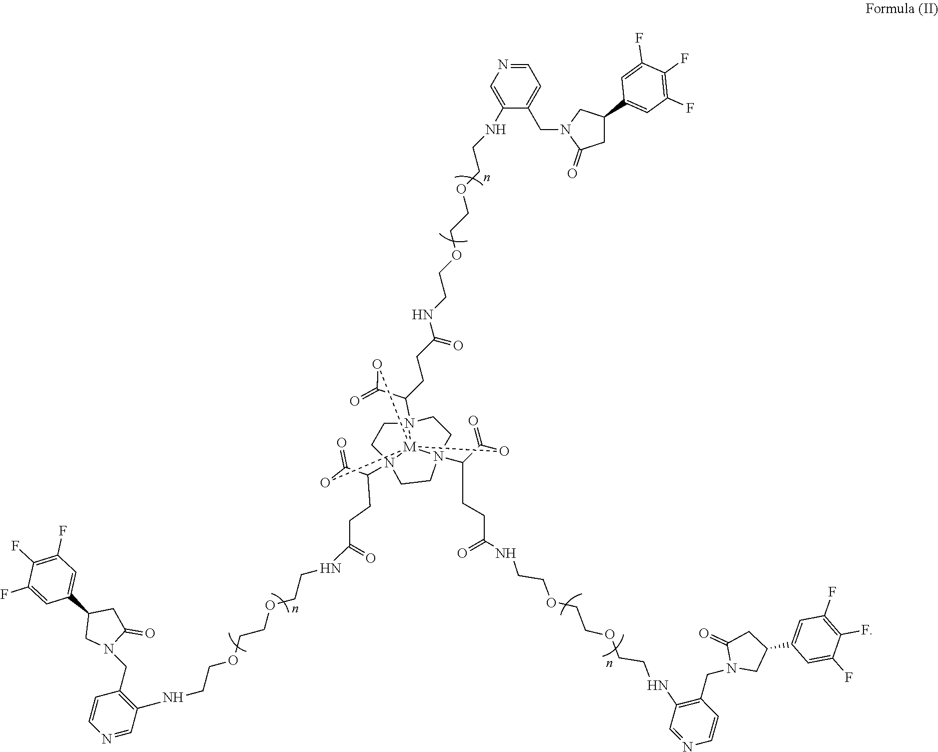

In another embodiment, A is a metal chelator selected from the group consisting of MeCOSar, DOTA, CB-TE2A, and NOTA; B is a peptide wherein one of the amino acid residues is lysine; C is AMTP; L₁ is absent; L₂ is a peptide linker comprising 2 to about 10 amino acid residues wherein one of the amino acid residues is lysine; L₃ is a polyethylene glycol linker comprising from 1 to 12 ethylene glycol repeating units; o is 0; p is 1; and m is 2; as shown in the compound of Formula (III):

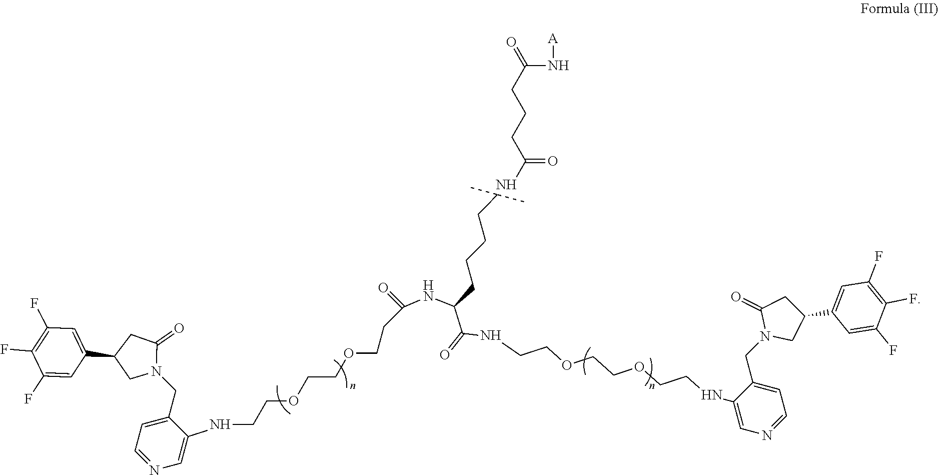

In yet another embodiment, A is a metal chelator selected from the group consisting of MeCOSar, DOTA, CB-TE2A, and NOTA; B is triazine; C is AMTP; L₁ is absent; L₂ and L₃ are polyethylene glycol linkers comprising from 1 to 12 ethylene glycol repeating units; o is 0; p is 1; and q is 2; as shown in the compound of Formula (IV):

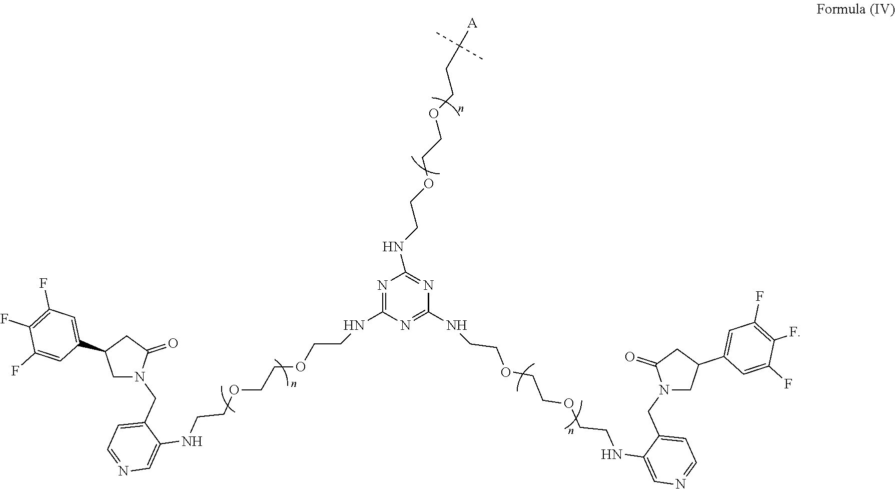

In still another embodiment, A is a metal chelator selected from the group consisting of MeCOSar, DOTA, CB-TE2A, and NOTA; B is a peptide; C is AMTP; L₁ is absent; L₂ is a peptide linker comprising 2 to about 10 amino acid residues wherein one of the amino acid residues is lysine and an albumin binding entity; L₃ is a peptide linker comprising 2 to about 10 amino acid residues wherein one of the amino acid residues is lysine; o is 0; p is 1; q is an integer from 1 to 6; and R₁ is H or an albumin-binding entity (p-iodophenyl derivative or evans blue moiety) as shown in the compound of Formula (V):

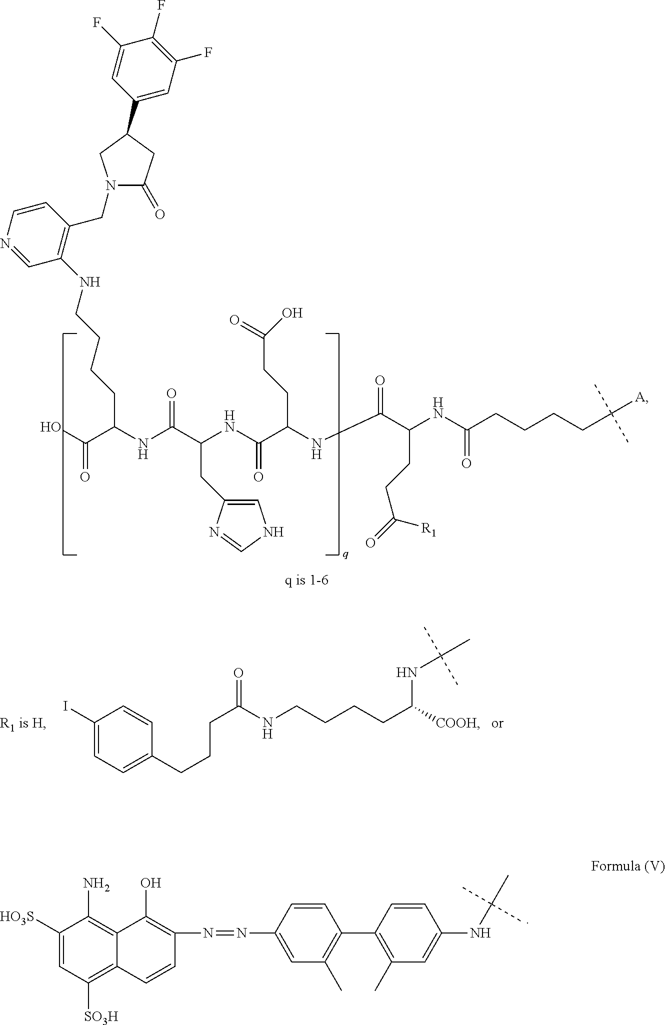

In yet another embodiment, A is a metal chelator selected from the group consisting of MeCOSar, DOTA, CB-TE2A, and NOTA; B is a peptide; C is AMTP; L₁ is absent; L₂ is a peptide linker comprising 2 to about 10 amino acid residues wherein one of the amino acid residues is lysine and an albumin binding entity; L₃ is a peptide linker comprising 2 to about 10 amino acid residues wherein one of the amino acid residues is lysine; o is 0; p is 1; q is an integer from 1 to 6; and R₁=H, an albumin-binding entity (p-iodophenyl derivative or evans blue moiety); as shown in the compound of Formula (VI):

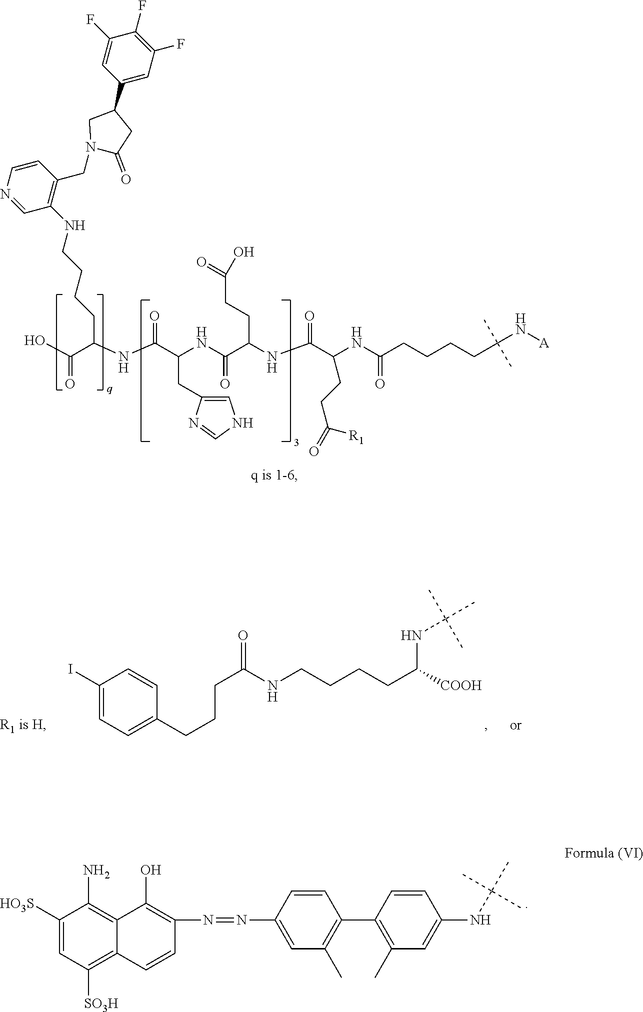

In another embodiment, A is a metal chelator selected from the group consisting of MeCOSar, DOTA, CB-TE2A, and NOTA; B is absent; C is absent; L₁ is an albumin binding entity and an ethylene glycol linker comprising from 1 to 12 ethylene glycol repeating units (depicted with 3 units); L₂ and L₃ are absent; o is 1; p is 0; and q is 0; as shown in the compound of Formula (VII):

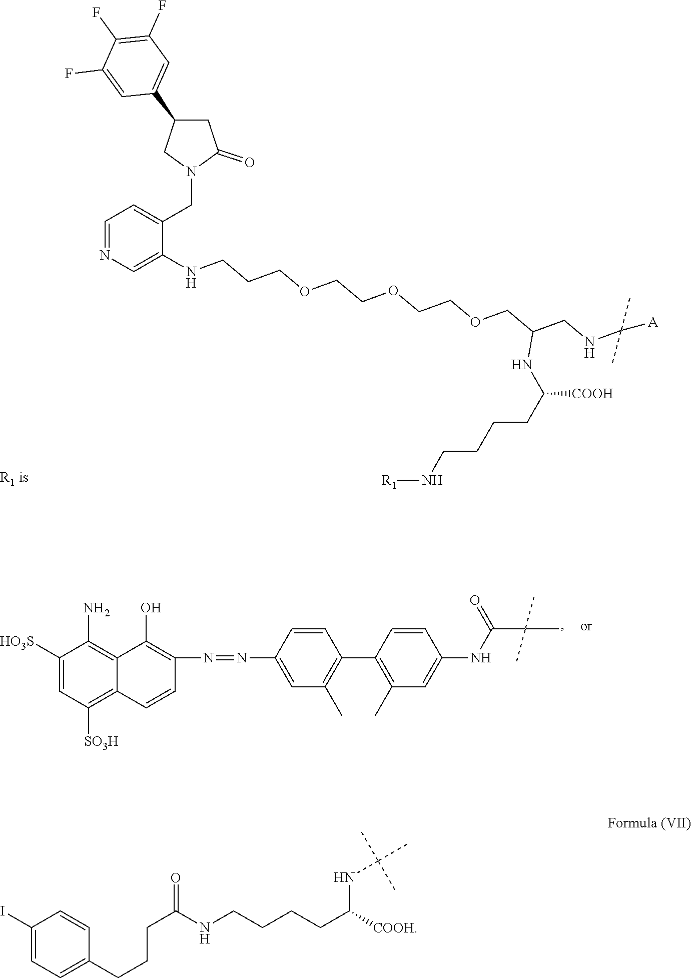

In still another embodiment, A is NOTA; B is triazine; C is AMTP; and L₁, L₂, and L₃ are linkers comprising from 1 to 12 ethylene glycol repeating units (depicted with 3 units); o is 2; p is 1; and q is 2; as shown in the compound of Formula (VIII):

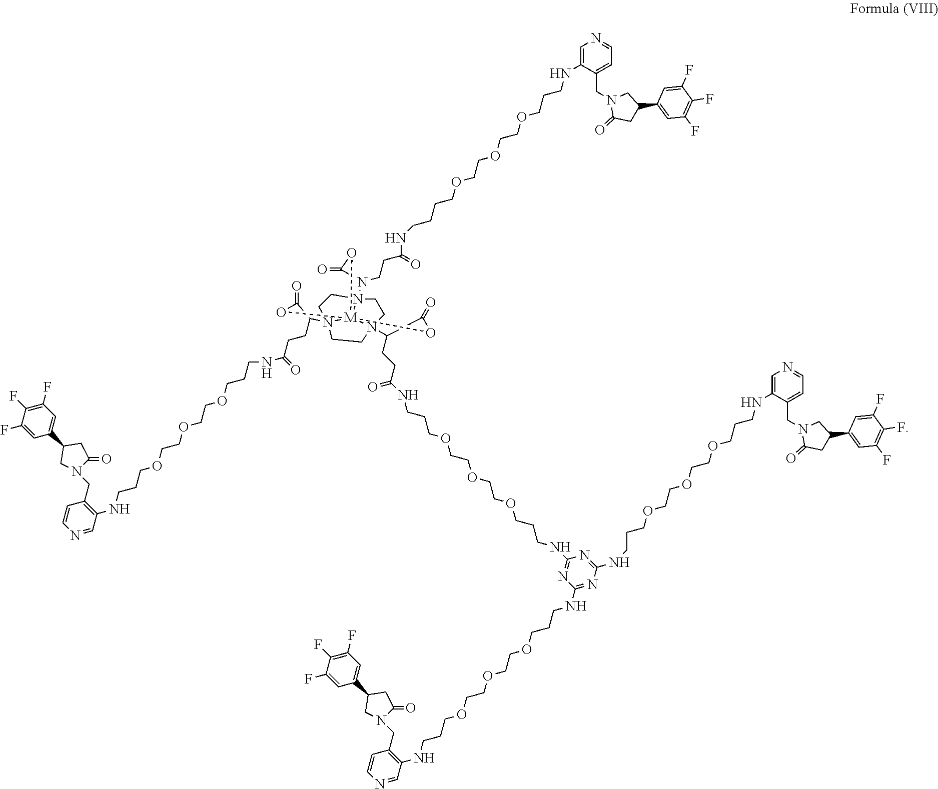

In yet another embodiment, A is HBED-CC; B is absent; C is absent; L₁ is a linker comprising from 1 to 12 ethylene glycol repeating unit (depicted with 3 units) and a peptide; L₂ and L₃ are absent; and o is 1; p is 0; and q is 0; as shown in the compound of Formula (IX):

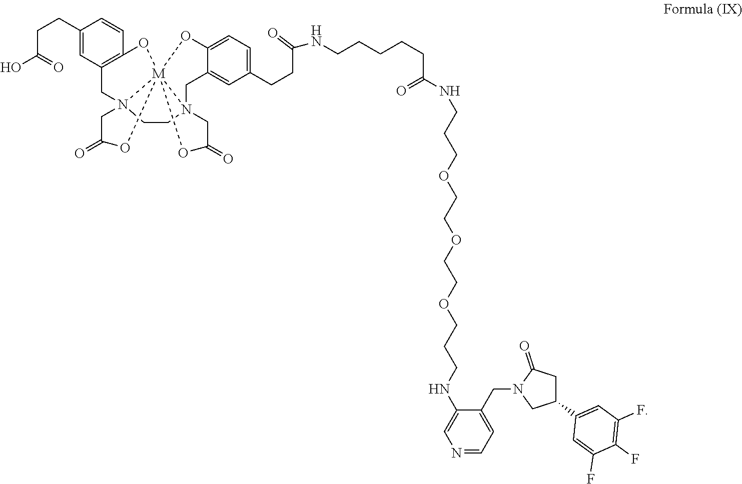

In still another embodiment, A is HBED-CC; B is absent; C is absent; L₁ is a linker comprising from 1 to 12 ethylene glycol repeating units and a peptide; L₂ and L₃ are absent; o is 2; p is 0; and q is 0; as shown in the compound of Formula (X):

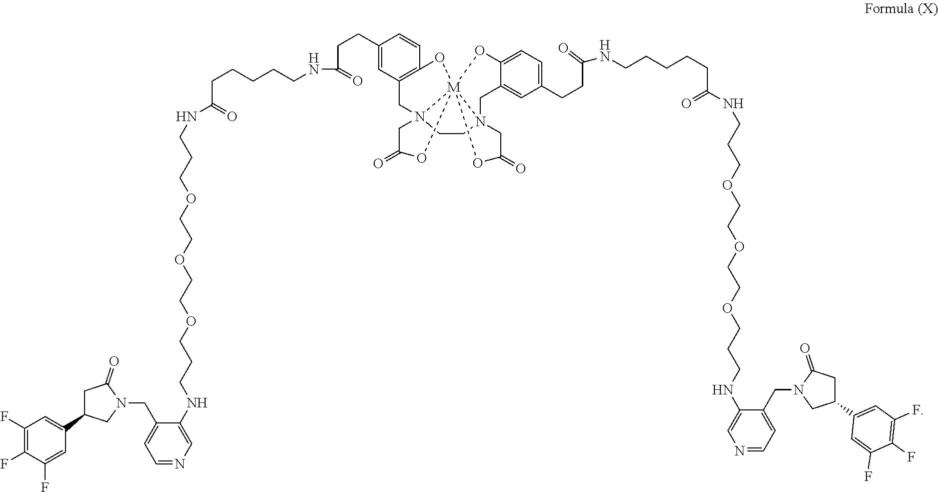

In yet another embodiment, A is DM1; B is absent; C is absent; L₁ is a linker comprising from 1 to 12 ethylene glycol repeating units (depicted with 3 units) and a disulfide group; L₂ and L₃ are absent; o is 1; p is 0; and q is 0; as shown in the compound of Formula (XI):

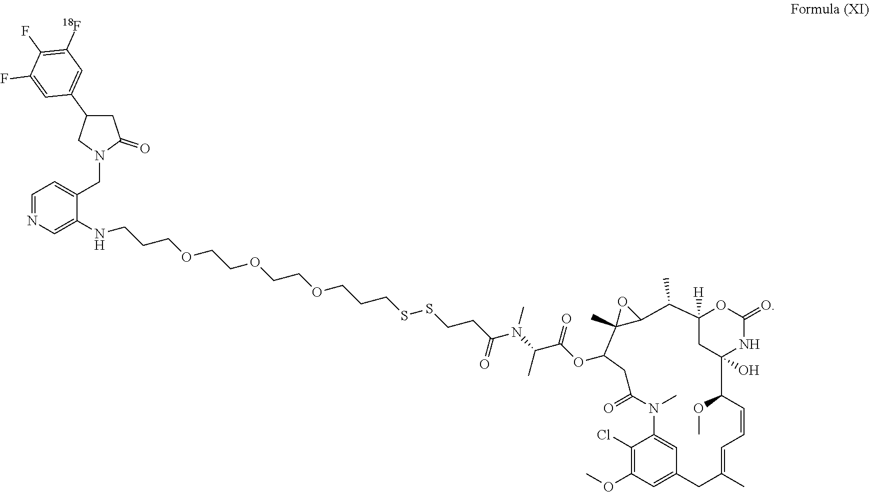

In still another embodiment, A is FTY720; B is absent; C is absent; L₁ is a polyethylene glycol linker comprising from 1 to 12 ethylene glycol repeating units (depicted with 3 units) and a disulfide group; L₂ and L₃ are absent; o is 1; p is 0; and q is 0; as shown in the compound of Formula (XII):

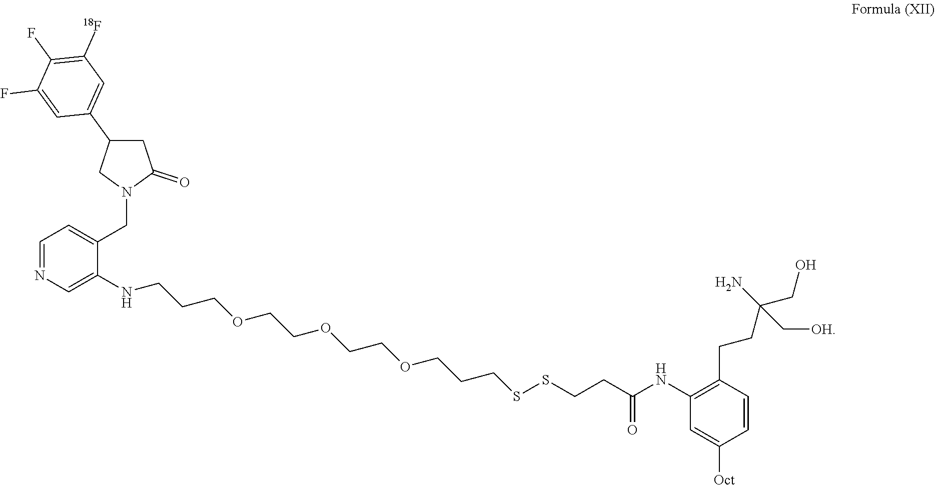

In another embodiment, A is NOTA; B is a peptide; C is DM1 or FTY720; L₁ is a polyethylene glycol linker comprising from 1 to 12 ethylene glycol repeating units; L₂ is a linker comprising from 1 to 12 ethylene glycol repeating units; L₃ is a disulfide linker; o is 2; p is 1; and q is 1; as shown in the compound of Formula (XIII):

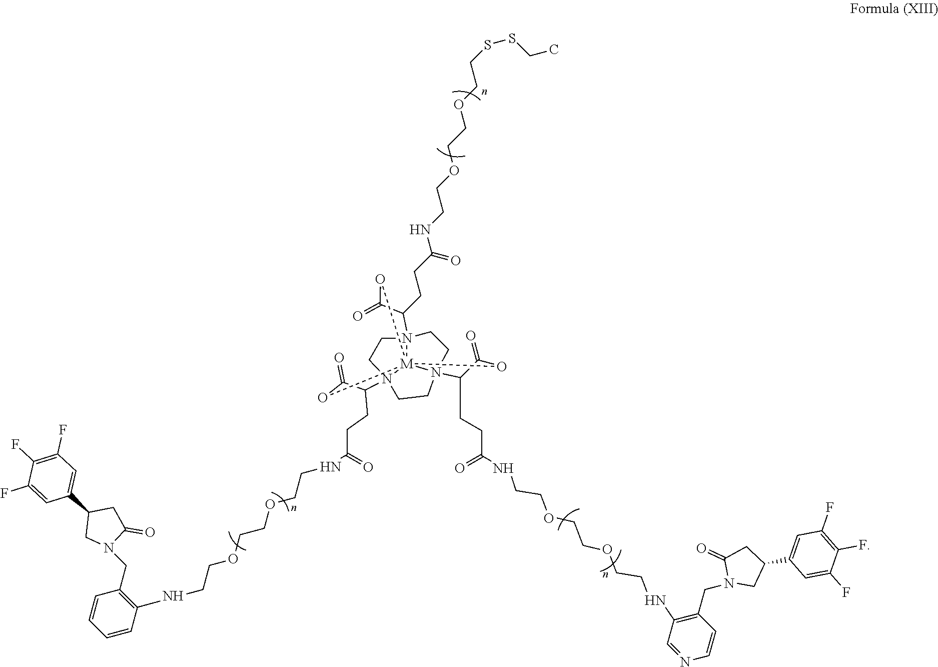

In yet another aspect, A is DM1 or FTY720; B is triazine; C is [¹⁸F]AMTP; L₁ is absent; L₂ is a linker comprising from 1 to 12 ethylene glycol repeating units and a disulfide group; L₃ is a linker comprising from 1 to 12 ethylene glycol repeating units; o is 2; p is 1; and q is 2; as shown in the compound of Formula (XIV):

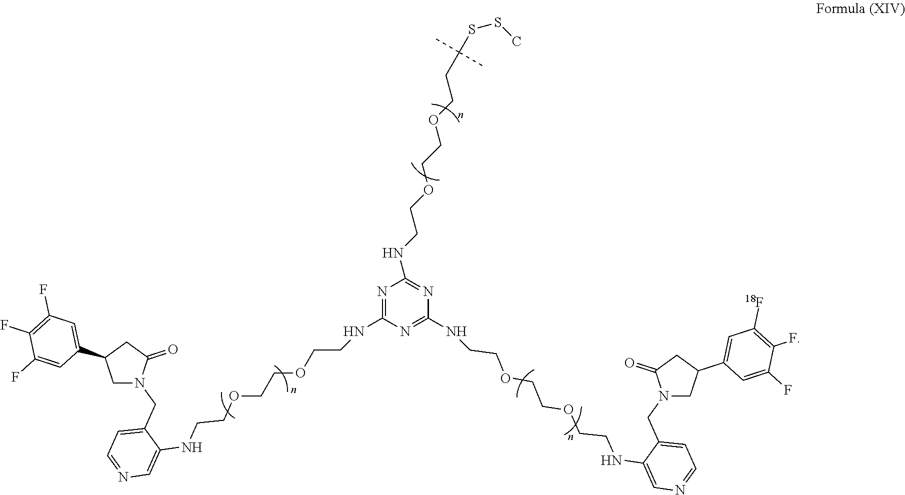

In yet another aspect, wherein the A is DM1 or FTY720; B is a peptide; C is AMTP; L₁ is absent; L₂ is a disulfide group and a peptide linker or a peptide linker, an albumin binding entity, and a disulfide group; L₃ is a peptide linker; o is 0; p is 1; q is an integer from 1 to 6; as shown in the compound of Formula (XV):

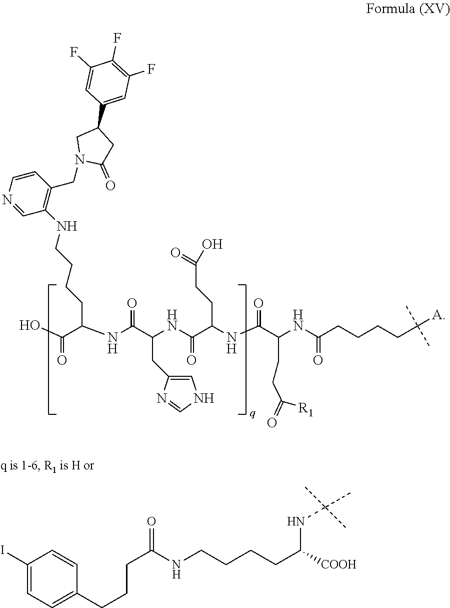

In general, the albumin binding moiety may comprise a fragment consisting of an azo dye Evans Blue fragment, a 4-(p-iodophenyl)butyryl fragment, a naphthalene acyl sulfonamide fragment, a diphenylcyclohexanol phosphate ester fragment, a 9-fluorenylmethooxycarbonyl fragment, a Fmoc derivative linked to a 16-sulfanylhexadecanoic acid, a dicoumarol fragment, a divalent diflunisal-indomethacin moiety linked through a γGlu-Lys dipeptide coupled to a unit of 8-amino-3,6-dioxaoctanoic acid (O2Oc) fragment, a lithocholic acid coupled to a γGlu linker fragment, a lithocholic acid coupled to a γGlu fragment, a 6-(4-(4-iodophenyl)butanamido)hexanoate coupled to carboxyfluorescein through a d-Lys fragment, a A083/B134 fragment, a A099/B344 fragment, a linear peptide 89D03: Ac-WWEQDRDWDFDVFGGGTP-NH₂ fragment, an acylated heptapeptide F-tag: fluorescein-EYEK(palmitate)EYE-NH2 fragment; a disulfide cyclized peptide SA21: Ac-RLIEDICLPRWGCLWEDD-NH₂ fragment; a head-to-tail cyclized peptide HSA-1: AK*K*PGK*AK*PG with variable lysine (K*) fragment, a bacterial ABD scaffold, human neonatal Fc receptor (FcRn), a bacterial protein Sso7d, a DARPin protein domain, a single-domain, fab domain, a nanobody (or a VNAR domain.

Generally, the polyamidoamine dendrimer is a generation 0 (G0) polyamidoamine dendrimer, a generation 1 (G1) polyamidoamine dendrimer, or a generation 2 (G2) polyamidoamine dendrimer.

In general, the metal chelator is incorporated to label the compounds with one radionuclide. Non-limiting examples of suitable radionuclides may be ⁶⁷Ga, ¹¹¹In, ^{99m}Tc, ¹³¹I, ¹²³I, ¹²⁵I, ¹⁰³Pd, ⁶⁸Ga, ¹⁸F, ⁶⁰Cu, ⁶¹Cu, ⁶²Cu, ⁶⁴Cu, ⁹⁰Y, ⁶⁷Cu, ¹⁸⁶Re, ¹⁸⁸Re, ¹⁹⁸Au, ¹⁵³Sm, ¹⁷⁷Lu, ²¹³Bi, ²¹²Pb, ²²³Ra, ²¹¹At, or ²²⁵Ac. In some embodiments, the radionuclide may be ⁶⁷Ga, or ⁶⁴Cu.

Generally, at least one fluorine of the compound of Formula (I) may comprise ¹⁸F.

The compound comprising Formula (I) may be a free form or a salt. When the compound is in a salt form, the salt is preferably a pharmaceutically acceptable salt. Pharmaceutically acceptable salts may include, without limitation, hydrochloride, hydrobromide, phosphate, sulfate, methanesulfonate, acetate, formate, tartaric acid, bitartrate, stearate, phthalate, hydroiodide, lactate, monohydrate, mucate, nitrate, phosphate, salicylate, phenylpropionate, isobutyrate, hypophosphite, maleic, malic, citrate, isocitrate, succinate, lactate, gluconate, glucuronate, pyruvate, oxalate, fumarate, propionate, aspartate, glutamate, benzoate, terephthalate, and the like. In other embodiments, the pharmaceutically acceptable salt includes an alkaline or alkaline earth metal ion salt. In particular, sodium, potassium or other pharmaceutically acceptable inorganic salts are used. The salt forms may be amorphous or in various polymeric forms including hydrates or solvates with alcohols or other solvents.

In general, the compounds of Formula (I), as disclosed above, are targeted to Synaptic Vesicle Glycoprotein 2A (SV2A). As such, the compounds of Formula (I) do not cross the blood brain barrier and maintains >90% intact in human serum at 37° C. for at least 4 hours.

II. Pharmaceutical Compositions comprising the Compound of Formula (I)

Another aspect of the present disclosure provides a pharmaceutical composition comprising the compound of Formula (I) and at least one pharmaceutically acceptable excipient or carrier.

A pharmaceutical composition of the disclosure comprises at least one pharmaceutically acceptable excipient. The pharmaceutically acceptable excipients are selected by those of skill in the art based upon the type of formulation.

In some embodiments, the compounds described herein may be administered intravenously (i.e., as a solution, suspension, or emulsion in a carrier).

In some embodiments, pharmaceutical compositions can include pharmaceutically acceptable carriers, excipients, and/or stabilizers are nontoxic to recipients at dosages and/or concentrations used to practice the methods disclosed herein. In some embodiments, pharmaceutically acceptable carriers, excipients, and/or stabilizers can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, pharmaceutical compositions herein formulated for intravenous administration can include one or more sterile liquids as pharmaceutically acceptable carriers. Non-limiting examples of sterile liquids suitable for use as pharmaceutically acceptable carriers herein can be water and oil, including those of petroleum, animal, vegetable, or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. In some embodiments, pharmaceutical compositions disclosed herein can be packaged in single unit dosages or in multi-dosage forms.

In some embodiments, pharmaceutical compositions herein suitable for intravenous administration can include aqueous and non-aqueous sterile injection solutions which can further contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH range from 3 to 9). The preparation of suitable intravenous formulations under sterile conditions is readily accomplished by standard pharmaceutical techniques well known to those skilled in the art.

In some embodiments, pharmaceutical compositions described herein can further include an anti-microbial agent. In accordance with these embodiments, the anti-microbial agent can, in an example, be an anti-viral, bactericidal agent, anti-fungal, or anti-bacterial agent. For example, the anti-microbial agent can be an anti-bacterial agent (antibiotic) such as doxycycline or other antibiotics such as a general antibiotic.

### III. Processes to Prepare Compounds Comprising Formula (I)

In another aspect, the present disclosure provides processes to prepare compounds of Formula (I). The processes commence by converting the (4R)-4-(3,4,5-trifluorophenyl)pyrrolidin-2-one to the (4R)-1-[(3-bromo-4-pyridyl)methyl]-4-(3,4,5-trifluorophenyl)pyrrolidin-2-one. (4R)-1-[(3-bromo-4-pyridyl)methyl]-4-(3,4,5-trifluorophenyl)pyrrolidin-2-one is coupled to a linker using an aqueous solution comprising copper powder producing a precursor. The precursor is coupled to either a metal chelator or highly potent chem drug through standard acyl coupling techniques known in the art. After deprotection, the SV2A-targeted theranostic agents for imaging and treatment of cancer innervation or neuroendocrine differentiation of cancer are produced. These processes are disclosed and known in the arts. These processes may utilize an acyl coupling agent, a proton acceptor, and at least one solvent. These processes may be conducted at various temperatures and pressures. Numerous processes are known by the skilled artisan and disclosed in the arts.

IV. Methods of Detecting and/or Treating Cancer

Another aspect of the present disclosure provides methods of treating and/or detecting cancer. The method comprises administering an effective amount of the compounds of Formula (I) or a composition comprising the compound of Formula (I) to a subject in need thereof wherein the subject in need thereof has or is suspected of one or more cancers.

As used herein, the term “detecting” refers to identifying the presence of a cancer. Various characteristics of the cancer may be measured (i.e., detected, determined, etc.). For example, the prevalence, volume, size, location, shape, position, etc., of the cancer may, but need not be, measured (i.e., detected, determined) using a variety of methods standard in the art.

As used herein, the term “treating” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein, “onset” or “occurrence” of a target disease or disorder includes initial onset and/or recurrence.

In certain embodiments, methods disclosed herein may be used for detecting cancer. Thus, in some aspects, the present disclosure provides methods for detecting cancer.

In certain embodiments, methods disclosed herein may be used for preventing, alleviating and/or treating cancer. Thus, in some aspects, the present disclosure provides methods for alleviating one or more symptoms and/or for treating cancer in a subject in need thereof by administration of any of the compounds disclosed herein, as well as a pharmaceutical composition comprising such.

In certain embodiments, methods disclosed herein may be used for detecting, preventing, alleviating and/or treating cancer. Thus, in some aspects, the present disclosure provides methods for detecting, alleviating one or more symptoms, and/or for treating cancer in a subject in need thereof by administration of any of the compounds disclosed herein, as well as a pharmaceutical composition comprising such.

To perform the methods disclosed herein, an effective amount of the compounds or compositions disclosed herein may be administered to a subject who needs treatment or detection of cancer via a suitable route (e.g., intravenous) at a suitable amount as disclosed herein or as would be appreciated by one of skill in the art.

The compounds disclosed herein may be administered as primary therapy, or as adjunct therapy, either following local intervention (surgery, radiation, local chemotherapy) or in conjunction with at least one other chemotherapeutic agent.

Suitable subjects may include, without limit, humans, as well as companion animals such as cats, dogs, rodents, and horses; research animals such as rabbits, sheep, pigs, dogs, primates, mice, rats, and other rodents; agricultural animals such as cows, cattle, pigs, goats, sheep, horses, deer, chickens, and other fowl; zoo animals; and primates such as chimpanzees, monkeys, and gorillas. The subject can be of any age without limitation. In an embodiment, the subject may be a human.

Generally, the compound of Formula (I) will be administered in a therapeutically effective amount which includes prophylactic amounts or lower dosages for example, when combined with another agent. As used herein, “an effective amount” refers to doses of compound sufficient to provide circulating or local concentrations high enough to impart a beneficial effect on the recipient thereof. The precise amount to be administered can be determined by the skilled practitioner in view of desired dosages, side effects, and medical history of the patient.

In some embodiments, a compound disclosed herein may be administered to a subject intravenously at least once a day, at least twice a day, at least three times a day or more.

The one or more cancers comprise one or more innervated cancers, one or more primary metastases, one or more cancers with neuroendocrine differentiation, or any combination thereof.

The one or more innervated cancers or one or more primary metastases comprises breast cancers, cervical cancers, colon cancer, gastric cancers, gliomas, head-and-neck cancers, melanomas, ovarian cancers, pancreatic cancers, prostate cancers, thyroid cancers, or any combination thereof.

The one or more cancers or one or more primary metastases with neuroendocrine differentiation comprises small-cell carcinomas, neoplasms, carcinoid, neuroendocrine carcinoma, large cell neuroendocrine carcinomas, prostate cancers, or any combination thereof.

### V. Methods of Imaging at Least on Region of the Subject's Body

In still another aspect of the present disclosure provides a method of imaging at least one region of a subject's body. The method comprises administering to the subject the compound of Formula (I) or the composition comprising the compound of Formula (I) and at least one pharmaceutically acceptable excipient or a carrier.

Suitable subjects are described in more detail in Section (IV). In one embodiment, the subject may be a human.

The method for detecting the compound of Formula (I) utilizes nuclear medicine imaging in at least one region of the body of the subject. Nuclear medicine imaging comprises the utilization of Single Photon Emission Computed Tomography (SPECT), Positron Emission Tomography (PET), CT, MRI, or a combination thereof. By using these types of nuclear medicine imaging, an accurate location and the extent of the cancer can be understood.

VI. Methods of Monitoring and/or Evaluating the Effectiveness of Treatment or Therapy for Cancer in a Subject's Body

In yet another aspect of the present disclosure provides a method of monitoring and/or evaluating the effectiveness of treatment or therapy for a cancer in a subject's body. The method comprises administering to the subject at least one compound of the compound of Formula (I) or a pharmaceutical composition comprising at least one pharmaceutically acceptable excipient or a carrier; detecting at least one compound of Formula (I) by PET or SPECT in at least one region of the body of the subject; and determining the level of SV2A in the at least one region of the body of the subject, and comparing it to a control reference from the subject before receiving the therapy or treatment; wherein, if the SV2A level in the at least one region of the body of the subject is lower than in the control reference, the treatment or therapy is at least partially effective for the subject.

Suitable subjects are described in more detail in Section (IV). In one embodiment, the subject may be a human.

The one or more innervated cancers, one or more primary metastases, one or more cancers with neuroendocrine differentiation, or any combination thereof are described in more detail in Section (IV).

The methods for detecting the one or more compounds of Formula (I) are described in more detail in Section (V). These methods further comprise performing a computed tomography (CT) scan.

The treatment or therapy for a cancer and/or primary and distal metastases comprises administration of one or more somatostatin analogs, chemotherapy, targeted therapy, immunotherapy, peptide receptor radionuclide therapy (PRRT), radiotherapy, or any combination thereof.

### V. Kits

In certain embodiments, kits are provided herein for use in detecting and/or treating cancer by use of a compound disclosed herein. In some embodiments, kits herein can include instructions for use in accordance with any of the methods described herein. In other embodiments, instructions can include a description of administering a compound and/or pharmaceutical composition disclosed herein to a subject at risk of cancer. In certain embodiments, kits disclosed herein can include instructions for using the components of the kit, for example relating to the use of a compound and/or pharmaceutical composition disclosed herein. In accordance with embodiments herein, kits can include instructions that provide information as to dosage, dosing schedule, and route of administration for the intended treatment.

In some embodiments, kits disclosed herein can include at least one container. In accordance with embodiments herein, containers can be any container such as tubes, vials, bottles, syringe, such as unit doses, bulk packages (e.g., multi-dose packages) or sub-unit doses. Instructions supplied in the kits of the invention can be written instructions on a label or package insert (e.g., a paper sheet included in the kit), but machine-readable instructions (e.g., instructions carried on a magnetic or optical storage disk) are also acceptable. The label or package insert indicates that the composition is used for detecting and/or treating cancer. Instructions can be provided for practicing any of the methods described herein.

Kits disclosed herein can include suitable packaging. Suitable packaging includes, but is not limited to, vials, bottles, jars, flexible packaging (e.g., sealed Mylar or plastic bags), and the like. Also contemplated herein are packages for use in combination with a specific device, such as an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle). At least one active agent in the composition can be a compound disclosed herein.

Kits can optionally provide additional components such as buffers and interpretive information. Normally, the kit includes a container and a label or package insert(s) on or associated with the container. In some embodiments, the invention provides articles of manufacture including contents of the kits described above.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described methods without departing from the scope of the invention, it is intended that all matter contained in the above description and in the examples given below, shall be interpreted as illustrative and not in a limiting sense.

### EXAMPLES

### Example 1: Conjugates Design of Radiotheranostics for Innervated Cancer by Repurposing Neuroimaging Agents on a Versatile Bifunctional Chelator Scaffold

Abstract: The SV2A targeting property of a neuroimaging agent for innervated cancer oncology was repurposed by incorporating lipophilic modification and overall structural modification. The radiotheranostic conjugates contain a metal chelating unit for imaging/therapy (e.g., ⁶⁴Cu/⁶⁷Cu) and an SV2A targeting ligand with a multivalent strategy for tumor targeting. The SV2A targeting ligand, (R)-1-((3-aminopyridin-4-yl)methyl)-4-(3,4,5-trifluorophenyl)pyrrolidin-2-one (AMTP), was conjugated with chelator 2,2′-(1,4,8,11-Tetraazabicyclo[6.6.2]hexadecane-4,11-di-yl)diacetic acid (CB-TE2A) through polyethylene glycol (PEG₃) linker to produce monovalent CB-TE2A-PEG₃-AMTP and bivalent CB-TE2A-(PEG₃-AMTP)₂ precursor conjugates. Small animal PET imaging in mice bearing SV2A^{high} H720 xenografts showed the tumor uptake of [⁶⁴Cu]Cu-CB-TE2A-PEG₃-AMTP peaked at 10 min post-injection (p.i.), followed by a rapid decline. In contrast, a gradually increasing profile was observed for [⁶⁴Cu]Cu-CB-TE2A-(PEG₃-AMTP)₂ until 40 min p.i., with a tumor to muscle ratio of 2.33. Further PET/CT images for [⁶⁴Cu]Cu-CB-TE2A-(PEG₃-AMTP)₂ in severe combined immunodeficiency (SCID) mice bearing dual tumor models (model 1: SV2A^{high} DU145-VC, SV2A^{low} DU145_sgPTP1B and model 2: SV2A^{high} PC3-VC, SV2A^{low} PC3_sgPTP1B) showed the SV2A selective tracer accumulation with extended tumor retention. The tumor to muscle ratios were 3.83 and 3.57 for model 1 and model 2, respectively, at 60 min p.i. The multivalent strategy revealed an augmented ligand-receptor binding reinforced by lipophilic modification to develop true chemically identical SV2A-targeted theranostic agents readily achievable by swapping ⁶⁴Cu with ⁶⁷Cu radionuclide.

Introduction: It is known that the heterogenous nature of tumor microenvironment (TME) provides a milieu of complex and inter-dependent elements, which serve the needs for tumor progression, angiogenesis, and metastasis, and in the meantime hijack the survival mechanisms of normal cells/organs to escape from immune surveillance and conventional treatments. To add another layer of complexity to the TME, a wealth of current literature reports has led to the premise that there exists a tumor-nerve crosstalk in the TME, which can activate nerve development and regenerative pathways to stimulate or enhance tumor growth.¹ While cancer innervation is often clinically presented in more aggressive or late-stage tumor phenotypes with poor prognosis,²⁻³ it also provides a novel target for us to develop paradigm-shifting therapies, which are in a dire need to enhance or replace the current treatments. As a matter of fact, the crosstalk between nerves, immune cells, and cancer cells within the TME likely underlies the resistance of tumors to current targeted therapies.¹

Radiopharmaceuticals that target neurotransmitter receptors/transporters, amyloid plaques, and neurofibrillary tangles have long been exploited for noninvasive assessment of neurodegenerative diseases via single photon emission tomography (SPECT) or positron emission tomography (PET).^{4, 5, 6} To date, a few radiopharmaceuticals have been approved by United States Food and Drug Administration (US-FDA) for neuropathological diagnosis (e.g., [¹⁸F]flortaucipir ([¹⁸F]-AV-1451),⁷ [¹⁸F]florbetaben ([¹⁸F]AV-1),⁸ [¹⁸F]florbetapir ([¹⁸F]AV-45),⁹ [¹⁸F]flutemetamol ([¹⁸F]FPIB)¹⁰) and many are in clinical trials (e.g., [¹¹C]PiB,¹¹ [¹⁸F]flutafuranol ([¹⁸F]AZD4694),¹² [¹⁸F]MK-3328,¹³ [¹⁸F]FIBT¹⁴). Given the wealth of these brain imaging agents in clinical practice and literature reports, targeted radiotheranostics were developed by presenting the well-validated targeting moieties on a versatile bifunctional chelator reported by us for innervated cancer, whose diagnosis and treatment represents an urgent unmet clinical need. In this work, a well-validated targeting moiety was chosen for synaptic vesicle glycoprotein 2 isoform A (SV2A) that has been reported in several PET imaging agents (e.g., ¹¹C-UCB-A,^{15 11}C-UCB-J,^{16 18}F-UCB-H,¹⁷ and ¹⁸F-SDM-8/SynVesT-1/2¹⁸⁻¹⁹) for noninvasive assessment of synaptic density, an essential functional indicator of the central nervous system. Indeed, SV2A has been reported in innervated cancers.²⁰⁻²¹ As such, a proof-of-concept study using ¹⁸F-SynVesT-1 was performed and demonstrated that SV2A-targeted PET imaging can be potentially used to detect neuroendocrine differentiation (NED) during the course of prostate cancer progression.²²

However, due to the physicochemical nature of ¹⁸F-SynVesT-1 as a neuroimaging agent, understandably, it cannot used without chemical modifications for PET imaging of NED in cancer. To repurpose a SV2A targeting neuroimaging agent for innervated cancer oncology while maintaining the SV2A targeting property the in vivo distribution of the radiopharmaceutical was rerouted to target the NED cancerous location over brain neurons. Therefore, a conjugate system was designed to endure the SV2A targeting moiety, (R)-1-((3-aminopyridin-4-yl)methyl)-4-(3,4,5-trifluorophenyl)pyrrolidin-2-one (AMTP, close structural similarity with UCB-J) on a bifunctional chelator scaffold, 2,2′-(1,4,8,11-Tetraazabicyclo[6.6.2]hexadecane-4,11-di-yl)diacetic acid (CB-TE2A). The major structural features of the conjugate include: (1) The bifunctional chelator scaffold (BFCS) enables labeling the conjugate with ⁶⁴Cu or ⁶⁷Cu, thus creates a chemically identical pair of radiotheranostics, (2) multivalent strategy²³ to have more than one AMTP ligand for augmented ligand-receptor binding, (3) a functionalized polyethylene glycol (PEG₃) linker for adaptable lipophilicity (Scheme 1).

Scheme 1 shows design of chemically identical pairs of radiotheranostic monovalent [^{64/67}Cu]Cu-CB-TE2A-PEG₃-AMTP and bivalent [^{64/67}Cu]Cu-CB-TE2A-(PEG₃-AMTP)₂ for SV2A-targeted PET imaging of cancer neuroendocrine differentiation when labeled with ⁶⁴Cu, and radiotherapy of innervated cancer when labeled with ⁶⁷Cu. Molecular moiety targeting synaptic vesicle glycoprotein 2 isoform A (SV2A): (R)-1-((3-aminopyridin-4-yl)methyl)-4-(3,4,5-trifluorophenyl)pyrrolidin-2-one (AMTP). PEG: polyethylene glycol, UCB-J: 2-Pyrrolidinone, 1-[(3-methyl-4-pyridinyl)methyl]-4-(3,4,5-trifluorophenyl)-, (4R)-, CB-TE2A: 2,2′-(1,4,8,11-Tetraazabicyclo[6.6.2]hexadecane-4,11-di-yl)diacetic acid.

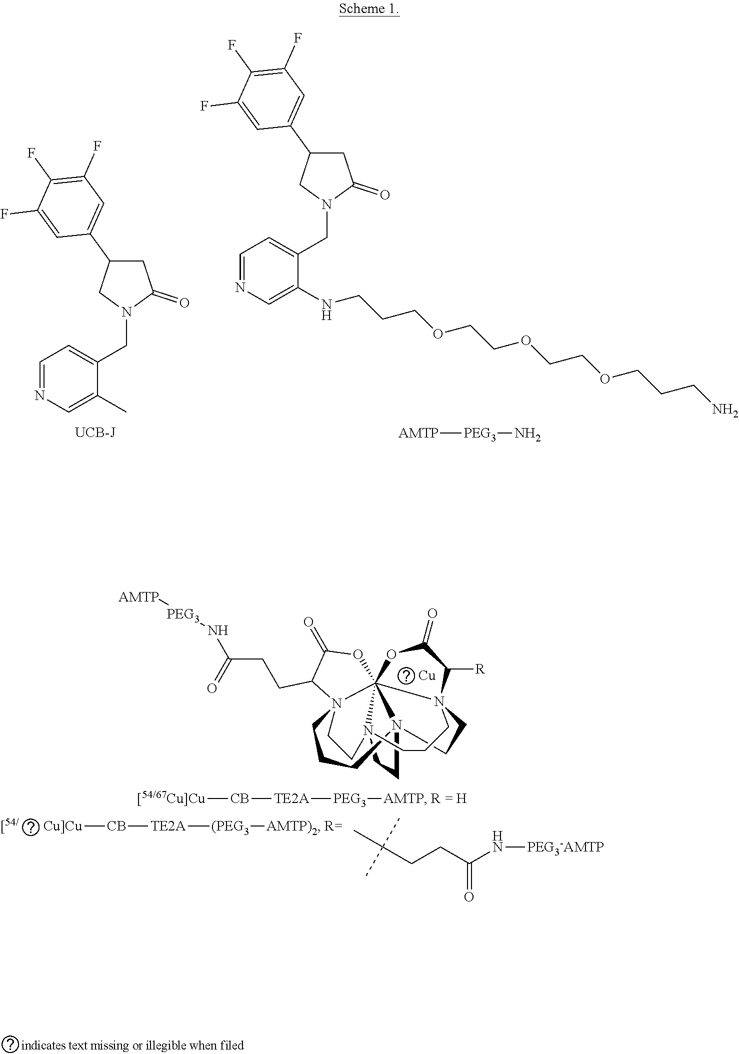

### Methods and Materials

General Materials and Procedures: All chemical reagents and solvents were obtained commercially (Fisher Scientific, Hampton, NH, USA; Sigma-Aldrich, St. Louis, MO, USA). Macrocyclics, Inc. supplied DOTA-mono-NHS tris (t-Bu ester). Unless otherwise specified, all reagents were used identically as received. A Millipore Gradient Milli-Q water system (Burlington, MA, USA) provided Milli-Q water for the preparation of all aqueous solutions. The synthesized conjugates and intermediate products were characterized by using an Agilent 6540 Accurate-Mass Quadrupole Time-of-Flight Liquid Chromatography-Mass Spectrometry (LC-MS) apparatus in combination with an Agilent 1290 ultra-performance liquid chromatography (UPLC) system (Santa Clara, CA, USA). A Varian 400 MHz spectrometer (Palo Alto, CA, USA) was used to record nuclear magnetic resonance (NMR) spectra. Purification of synthesized compounds was accomplished with an Agilent 1260 Infinity preparative high-performance liquid chromatography (HPLC) system outfitted with a 1260 photodiode array detector (PDA) and an Agilent Prep-C18 column (150×21.2 mm, 5 μm) (Santa Clara, CA, USA). An Agilent 1220 Infinity II analytical HPLC system (76337 Waldbronn, Germany) equipped with a 1220 LC diode array detector (DAD), an in-line Eckert & Ziegler radio detector (Eckert & Ziegler Radiopharma, Inc. Hopkinton, MA, USA), and a C18 Atlantis® T3 column (4.6×250 mm, 5 μm) (Waters, Ireland) were utilized in order to characterize the radiolabeled compounds.

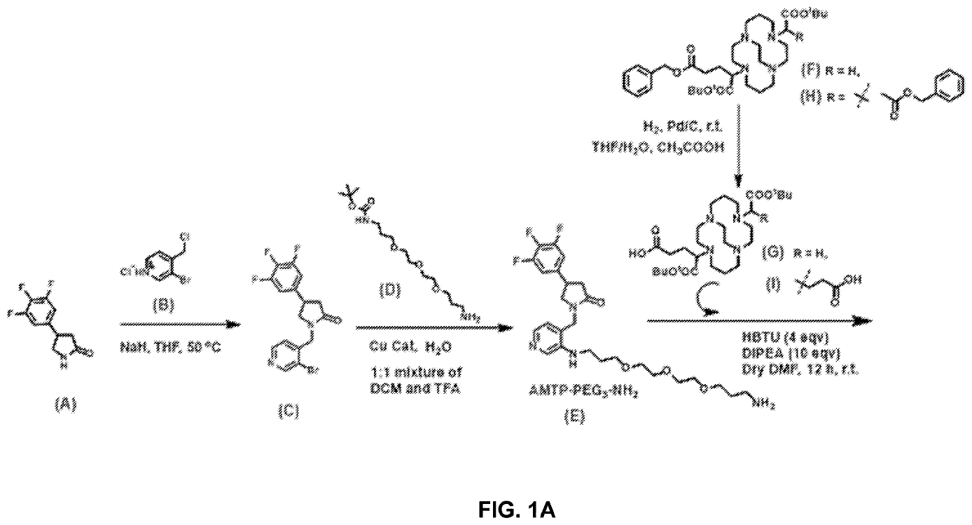

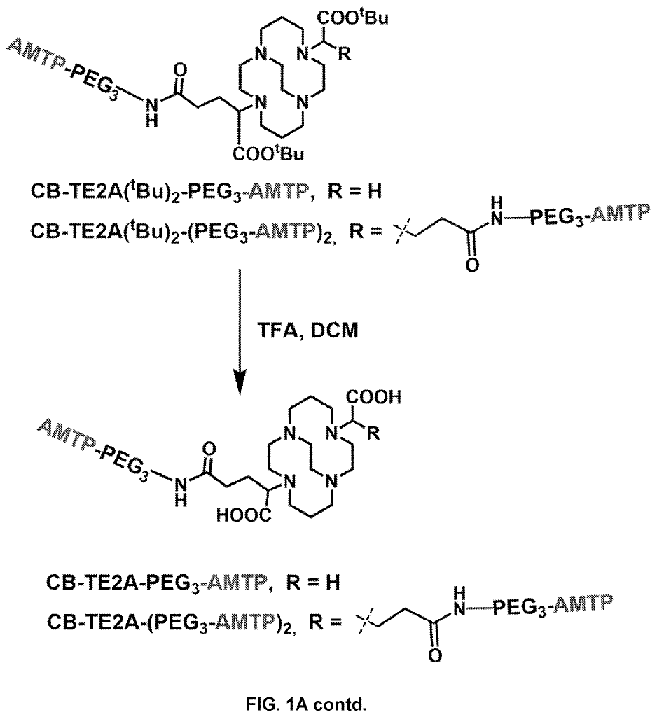

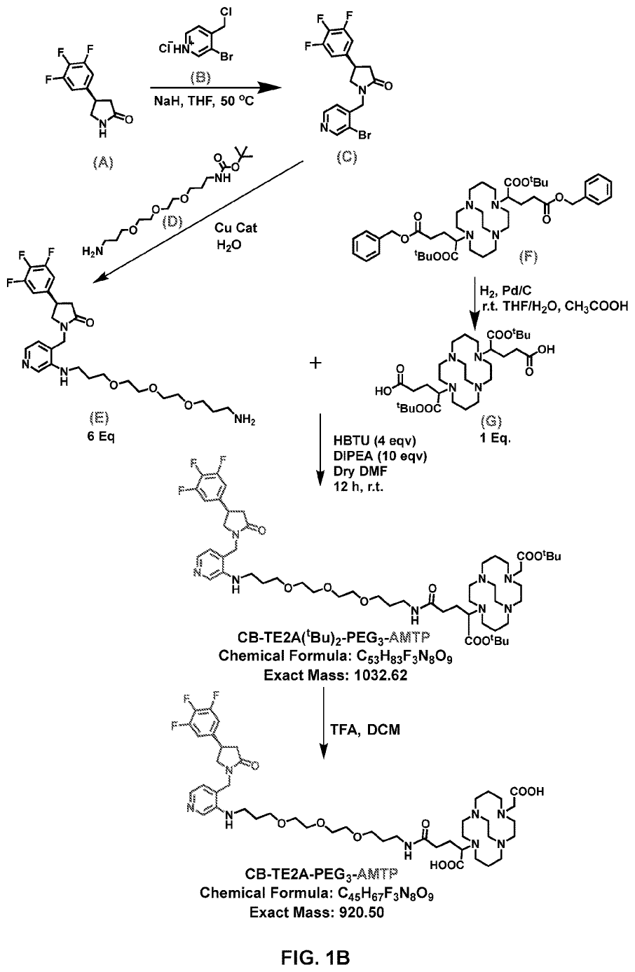

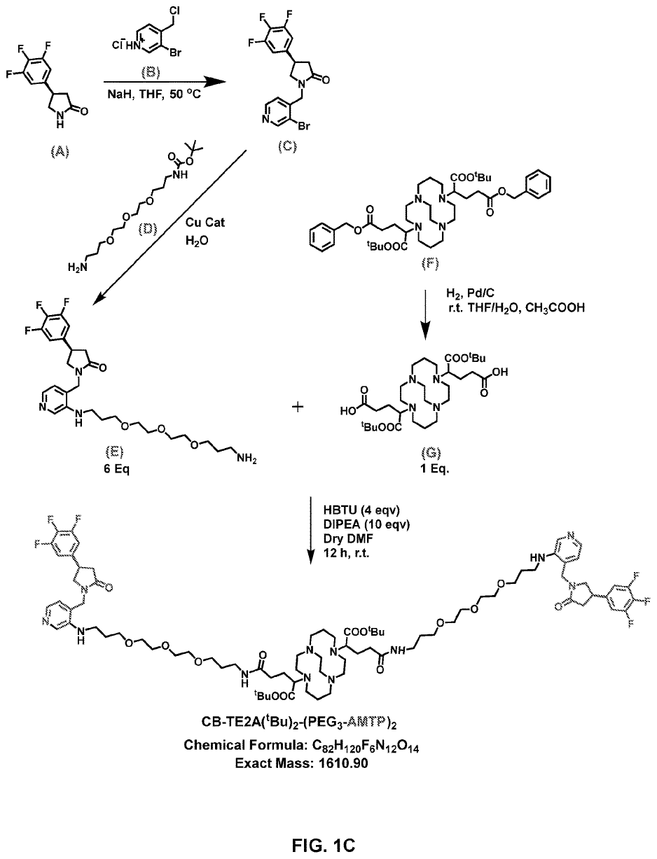

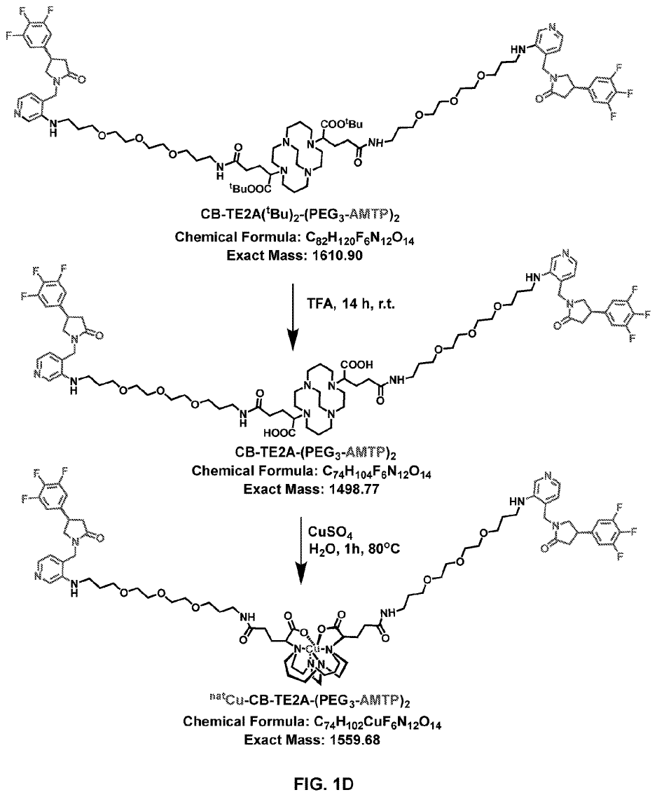

Synthesis of the Monovalent and Bivalent Conjugates: Among all the reported SV2A-specific PET radiotracers, [¹¹C]UCB-J¹⁶ and [¹⁸F]UCB-J²⁴ have shown excellent performance in synaptic density imaging. Both tracers have been employed to investigate a variety of neurodegenerative and neuropsychiatric disorders. To explore the UCB-J core further, its targeting was leveraged behavior into a small molecule drug conjugate (SMDC) platform. The methyl group in the UCB-J core was replaced with an amine to enable linker functionalization and regulate lipophilicity for uninterrupted or improved SV2A targeting properties irrespective of tumor location. The linker modification allows us to incorporate a chelating ligand for radiometalation (^{64/67}Cu) to wing up the theranostic efficacy. Synthesis of the precursor molecules has been accomplished according to FIG. 1A and described below.

In brief, commercially available (4R)-4-(3,4,5-trifluorophenyl)pyrrolidin-2-one (compound A) was coupled with 3-bromo-4-(chloromethyl)pyridine hydrochloride in the presence of sodium hydride at 50° C. to produce (4R)-1-[(3-bromo-4-pyridyl)methyl]-4-(3,4,5-trifluorophenyl)pyrrolidin-2-one (compound C) with 36% yield. Tert-butyl (3-(2-(2-(3-aminopropoxy) ethoxy) ethoxy) propyl) carbamate (compound D) was then treated with compound C in presence of aqueous copper powder at 96° C. for 36 hrs. The reaction mixture was then filtered and concentrated under vacuum. The crude mixture was treated with 200 μL 1:1 mixture of DCM and TFA for 10 minutes at room temperature to produce AMTP-PEG₃-NH₂ (compound E) with 49% yield. A significant amount of dibrominated and unreacted compound C (m/z 307.12 and 385.02) was isolated in the crude reaction mixture, confirmed by mass spectrometry. No significant alteration in product formation was observed after 72 hrs. of reaction at 96° C. followed by TFA treatment. Increasing temperature resulted in an increased rate of debromination.

The synthesis of compounds G and I were accomplished according to our previously reported procedure.²⁵ The Intermediate AMTP-PEG₃-NH₂ was reacted with corresponding chelators catalyzed by HBTU followed by TFA-mediated deprotection to produce monovalent CB-TE2A-PEG₃-AMTP and bivalent CB-TE2A-(PEG₃-AMTP)₂ conjugates. Although the bivalent conjugate CB-TE2A-(PEG₃-AMTP)₂ showed a meager yield (~6% combined steps 3 and 4) compared to monovalent CB-TE2A-PEG₃-AMTP (14%), it could be enhanced by further optimizing the reaction conditions and by increasing the stoichiometry of the reactants. This adaptable approach could synthesize several distinct theranostics, depending on the connecting linker and the tumor-specific targeting ligands.

The details of the syntheses are provided below.

### Preparation of CB-TE2A-(PEG₃-AMTP)₂ SV2A Conjugates

The preparation of CB-TE2A-(PEG₃-AMTP)₂ SV2A conjugates was accomplished through a multi-step synthesis as shown in FIGS. 1B and 1C.

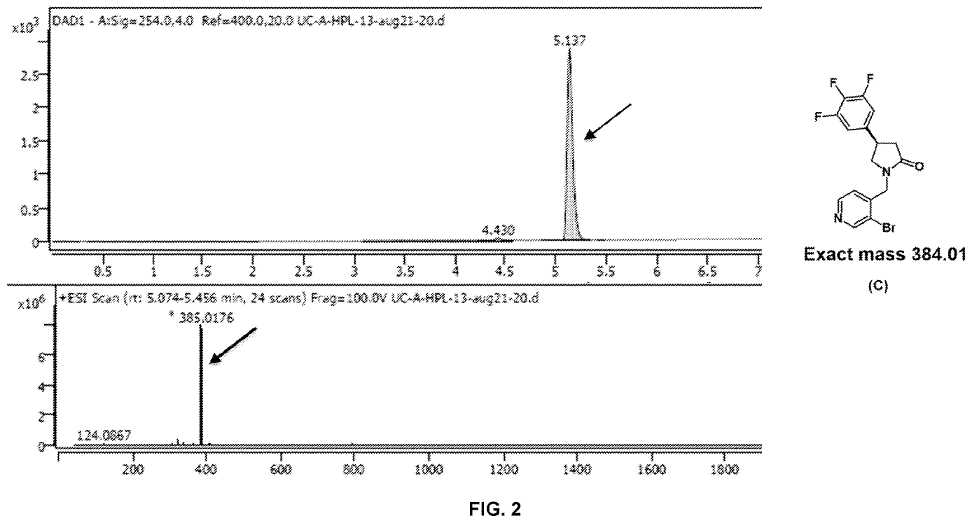

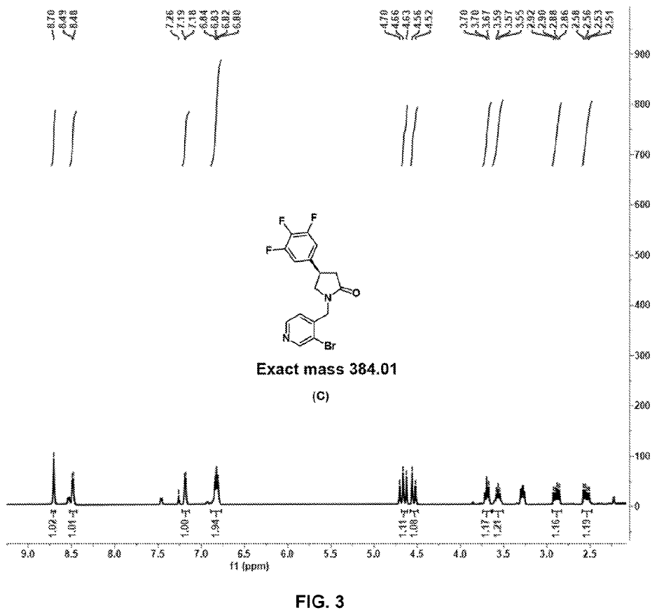

Synthesis of (4R)-1-[(3-bromo-4-pyridyl)methyl]-4-(3,4,5-trifluorophenyl)pyrrolidin-2-one, Compound C. Compound C was synthesized according to the previously reported procedure.⁵² In brief, in a round-bottom flask, (4R)-4-(3,4,5-trifluorophenyl)pyrrolidin-2-one (compound A, 60 mg, 0.28 mmol, 1 eqv.) was added to a solution of 3-bromo-4-(chloromethyl)pyridine hydrochloride (compound B, 81 mg, 0.34 mmol, 1.2 eqv.) in anhydrous THF (4 ml). After cooling down to 0° C., sodium hydride, 60% dispersion in mineral oil (28 mg, 0.84 mmol, 3 eq.) was added in small portions and the reaction mixture was stirred for 16 hrs. at 50° C. until complete conversion. The reaction mixture was then diluted with ethyl acetate (50 ml), brine (20 ml), followed by addition of charcoal, and the resulting mixture was filtered through a celite bed. Extracted organic layer was dried over anhydrous sodium sulfate and then concentrated under vacuum. The resulting crude product was purified by silica gel column chromatography to afford (4R)-1-[(3-bromo-4-pyridyl)methyl]-4-(3,4,5-trifluorophenyl)pyrrolidin-2-one C (38 mg, 36% yield). MS (ESI) m/z calcd for C₁₆H₁₂BrF₃N₂O: 384.01; found: 385.01 ([M+H]⁺ (FIG. 2). ¹H NMR (400 MHz, CDCl₃): δ 8.70 (s, 1H), 8.49 (d, 1H), 7.19 (d, 1H), 6.87-6.75 (m, 2H), 4.66 (d, 1H), 4.56 (d, 1H) 3.72-3.66 (m, 1H), 3.60-3.52 (m, 1H), 2.92-2.85 (m 1H), 2.59-2.50 (m, 1H), FIG. 3.

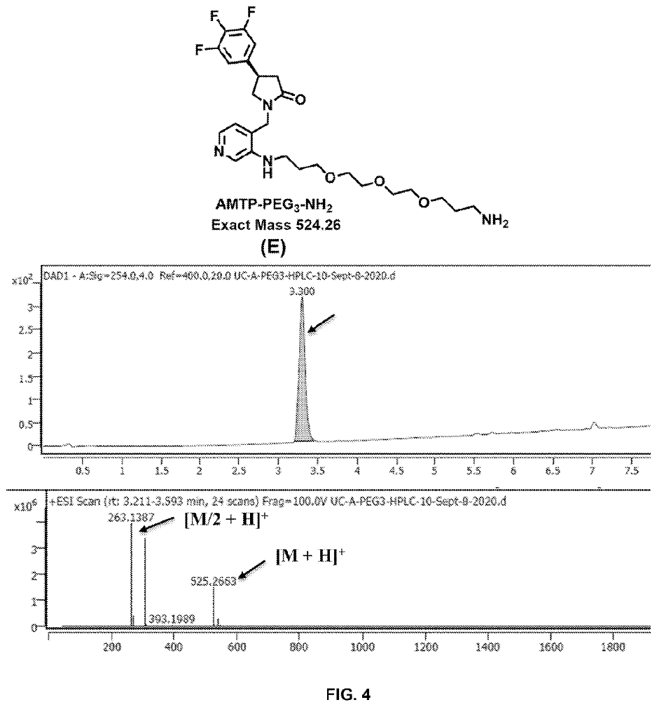

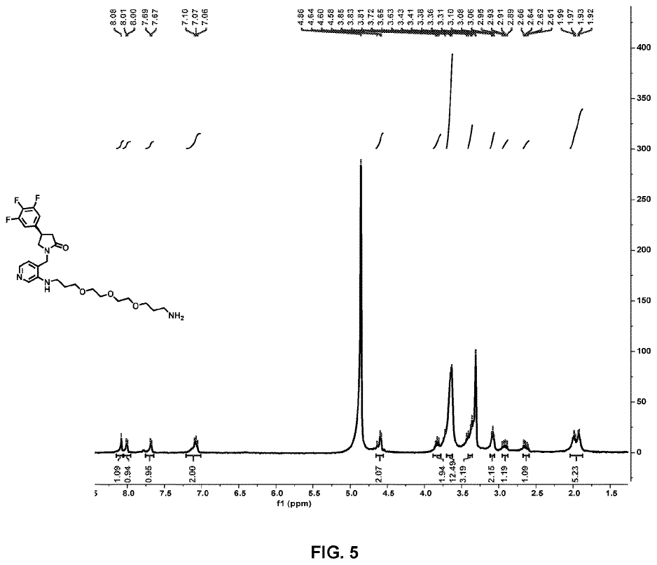

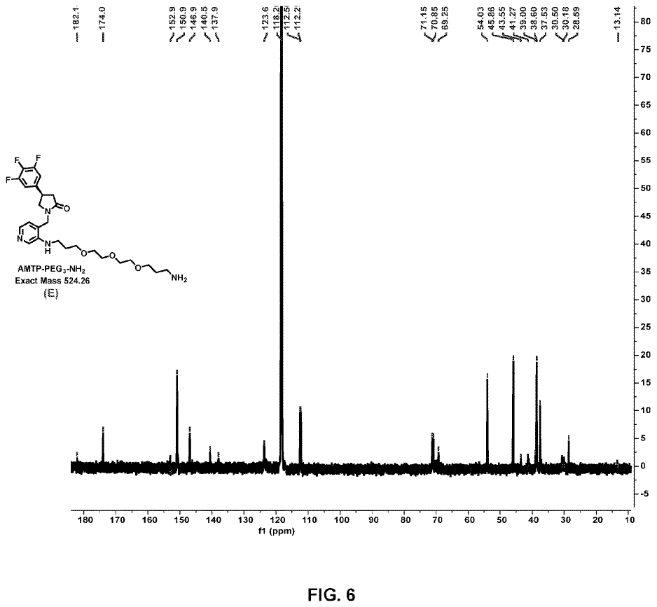

Synthesis of AMTP-PEG₃-NH₂ Compound E: In a round-bottom flask, compound C (38 mg, 0.1 mmol 1 eqv.), tert-butyl (3-(2-(2-(3-aminopropoxy) ethoxy) ethoxy) propyl) carbamate D (640 mg liquid, 2 mmol, 20 eqv.), Cu powder (1 mg) and water (400 μL) were heated at 96° C. for 36 hrs. The reaction mixture was then filtered through a nylon syringe filter (target2 nylon syringe filter 0.2 μm, 17 mm) and concentrated under vacuum. The concentrated reaction mixture was treated with 200 μL 1:1 mixture of DCM and TFA for 10 minutes at room temperature. The product AMTP-PEG₃-NH₂ formation was confirmed by mass spectrometry and further purified by HPLC (0 min: 20% CH₃CN, 80% H₂O; 15 min: 70% CH₃CN, 30% H₂O; flow of 20 mL/min; all solvents contained 0.1% TFA) to produce 26 mg pure sticky liquid, yield 49%. MS (ESI) m/z calcd for C₂₆H₃₅F₃N₄O₄: 524.26; found: 525.26 ([M+H]⁺, 263.13 [M/2+H]⁺) (FIG. 4). ¹H NMR (400 MHz, CD₃OD): δ 8.08 (s, 1H), 8.04-7.96 (m, 1H), 7.74-7.64 (m, 1H), 7.19-7.01 (m, 2H), 4.64-4.55 (m, 2H), 3.88-3.78 (m, 2H), 3.70-3.61 (m, 12H), 3.41-3.35 (m, 3H) 3.12-3.05 (m, 2H), 2.95-2.87 (m, 1H), 2.67-2.59 (m 1H), 2.04-1.87 (m, 5H), FIG. 5. ¹³C NMR (100 MHz, CD₃CN) δ 182.14, 174.01, 152.93, 150.91, 146.91, 140.58, 137.95, 123.67, 112.50, 112.29, 71.15, 70.85, 69.25, 54.03, 45.86, 43.55, 41.27, 39.00, 38.60, 37.53, 30.50, 30.18, 28.59, 13.14, FIG. 6.

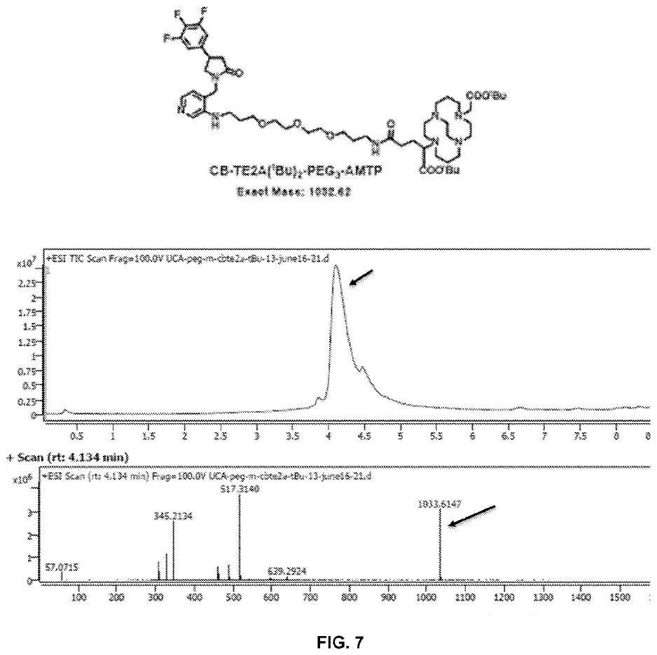

Synthesis of CB-TE2A(^{t}Bu)₂-PEG₃-AMTP: 5-(tert-butoxy)-4-(11-(2-(tert-butoxy)-2-oxoethyl)-1,4,8,11-tetraazabicyclo[6.6.2]hexadecan-4-yl)-5-oxopentanoic acid (3 mg, 0.006 mmol) was dissolved in THF (200 μL) and stirred for 5 min under nitrogen atmosphere. Compound E (2.5 mg, 0.005 mmol) was dissolved in DMF (300 μL) and added to the former reaction vial at room temperature. DIPEA (10 μL) and HBTU (7 mg) were added to the reaction mixture and agitated for 12 hrs. The crud mixture was purified by HPLC (0 min: 20% CH₃CN, 80% H₂O; 18 min: 60% CH₃CN, 40% H₂O; flow of 20 mL/min; all solvents contained 0.1% TFA) to produce CB-TE2A(^{t}Bu)₂-PEG₃-AMTP, 2 mg, yield 40%. MS (ESI) m/z calcd for C₅₃H₈₃F₃N₈O₉: 1032.62; found: 1033.64 ([M+H]⁺, 517.33 [M/2+H]⁺, 345.23 [M/3+H]⁺) (FIG. 7).

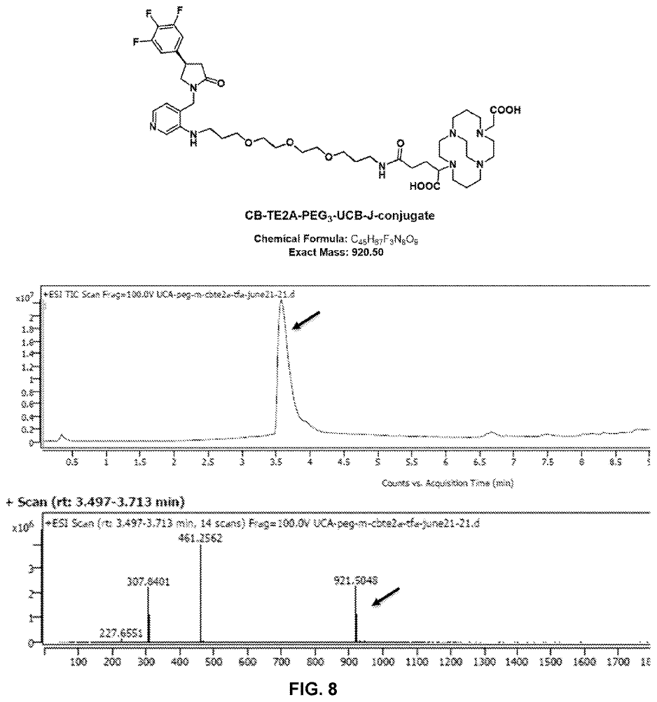

Synthesis of CB-TE2A-PEG₃-AMTP: Conjugate CB-TE2A(^{t}Bu)₂-PEG₃-AMTP (2 mg, 0.002 mmol) was treated with 80% trifluoroacetic acid (400 μL TFA with 100 μl DCM) for 12 hrs. to produce the CB-TE2A-PEG₃-AMTP precursor molecule and was purified by HPLC (0 min: 10% CH₃CN, 90% H₂O; 20 min: 50% CH₃CN, 50% H₂O; flow of 20 mL/min; all solvents contained 0.1% TFA). 0.6 mg lyophilized product was obtained with 34% reaction yield. MS (ESI) m/z calcd for C₄₅H₆₇F₃N₈O₉: 920.50; found: 921.50 [M+H]⁺, 461.25 [M/2+H]⁺, 307.84 [M/3+H]⁺ (FIG. 8). FIG. 1B shows the specific reaction scheme to prepare CB-TE2A-PEG₃-AMTP.

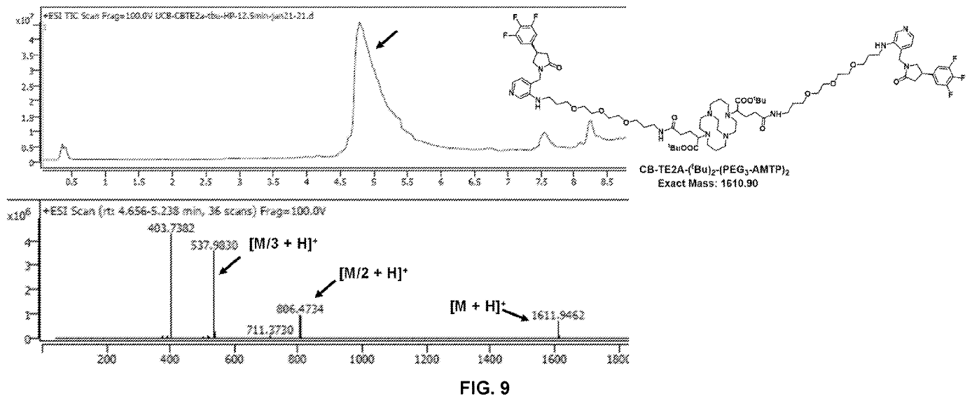

Synthesis of CB-TE2A(^{t}Bu)₂-(PEG₃-AMTP)₂: 4,4′-(1,4,8,11-tetraazabicyclo[6.6.2]hexadecane-4,11-diyl)bis(5-tert-butoxy-5-oxopentanoic acid) (3 mg, 0.005 mmol) was dissolved in THF (200 μL) and stirred for 5 min under nitrogen atmosphere. Compound E (10.5 mg, 0.02 mmol) was dissolved in DMF (400 μL) and added to the former reaction vial at room temperature. DIPEA (10 μL) and HBTU (8 mg) were added to the reaction mixture and agitated for 12 hrs. The crud mixture was purified by HPLC (0 min: 20% CH₃CN, 80% H₂O; 18 min: 60% CH₃CN, 40% H₂O; flow of 20 mL/min; all solvents contained 0.1% TFA) to produce CB-TE2A(^{t}Bu)₂-(PEG₃-AMTP)_{2, 4} mg, yield 49%. MS (ESI) m/z calcd for C₈₂H₁₂₀F₆N₁₂O₁₄: 1610.90; found: 1611.94 [M+H]⁺, 806.47 [M/2+H]⁺, 537.98 [M/3+H]⁺ (FIG. 9).

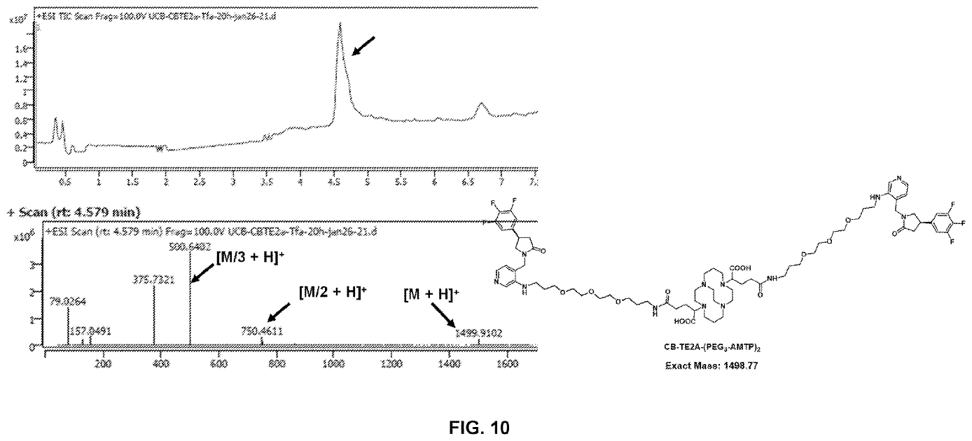

Synthesis of CB-TE2A-(PEG₃-AMTP)₂: Conjugate CB-TE2A(^{t}Bu)₂-(PEG₃-AMTP)₂ (4 mg, 0.0025 mmol) was treated with 90% trifluoroacetic acid (900 μL TFA with 100 μl DCM) for 16 hrs. to produce the CB-TE2A-(PEG₃-AMTP)₂ precursor molecule and was purified by HPLC (0 min: 10% CH₃CN, 90% H₂O; 20 min: 50% CH₃CN, 50% H₂O; flow of 20 mL/min; all solvents contained 0.1% TFA). 0.5 mg lyophilized product was obtained with 13% reaction yield. MS (ESI) m/z calcd for C₇₄H₁₀₄F₆N₁₂O₁₄: 1498.77; found: 1499.91 [M+H]⁺, 750.46 [M/2+H]⁺, 500.64 [M/3+H]⁺, 375.73 [M/4+H]⁺ (FIG. 10). FIG. 1C shows the specific reaction scheme to prepare CB-TE2A-(PEG₃-AMTP)₂.

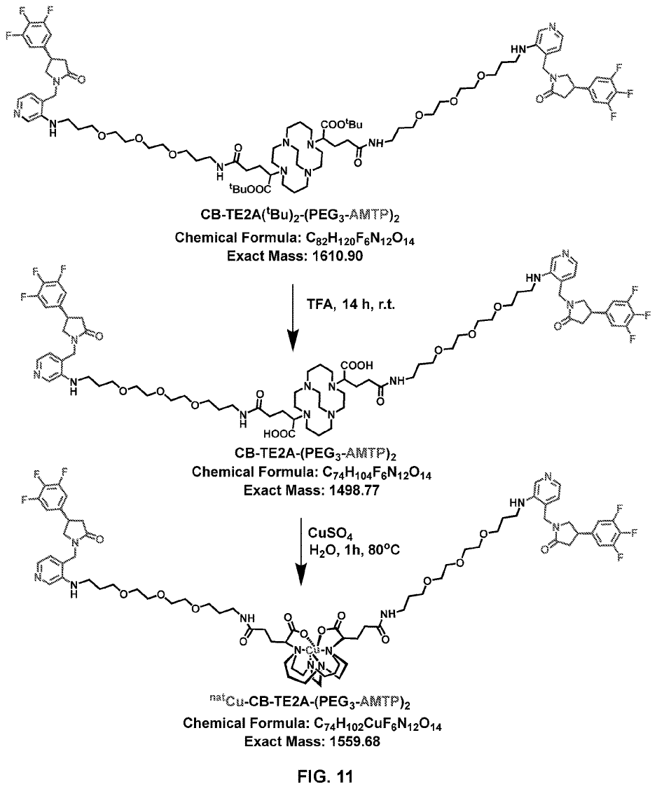

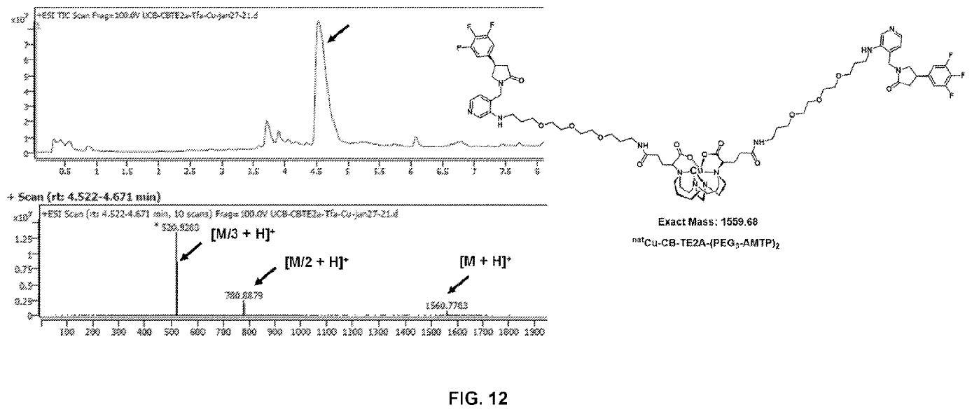

Synthesis of ^{nat}Cu-CB-TE2A-(PEG₃-AMTP)₂: CB-TE2A-(PEG₃-AMTP)₂ (0.5 mg, 0.001 mmol) was treated with aqueous CuSO₄ solution at room temperature for 30 min and the product formation was confirmed by mass spectrometry. The crud mixture was purified by HPLC (0 min: 15% CH₃CN, 85% H₂O; 15 min: 45% CH₃CN, 55% H₂O; flow of 20 mL/min; all solvents contained 0.1% TFA) to produce ^{nat}Cu-DOTA-(PEG₃-AMTP)₂ as white solid, 0.6 mg, yield 38%. MS (ESI) m/z calcd for C₇₄H₁₀₂CuF₆N₁₂O₁₄: 1559.68; found: 1560.77 [M+H]⁺, 780.88 [M/2+H]⁺, 520.92 [M/3+H]⁺ (FIG. 12). FIG. 11 shows the specific reaction scheme to prepare CB-TE2A-(PEG₃-AMTP)₂.

Radiochemistry: Production of ⁶⁴Cu was accomplished according to our previously reported procedure at cyclotron and radiochemistry facility at UTSouthwestern medical center.⁵³

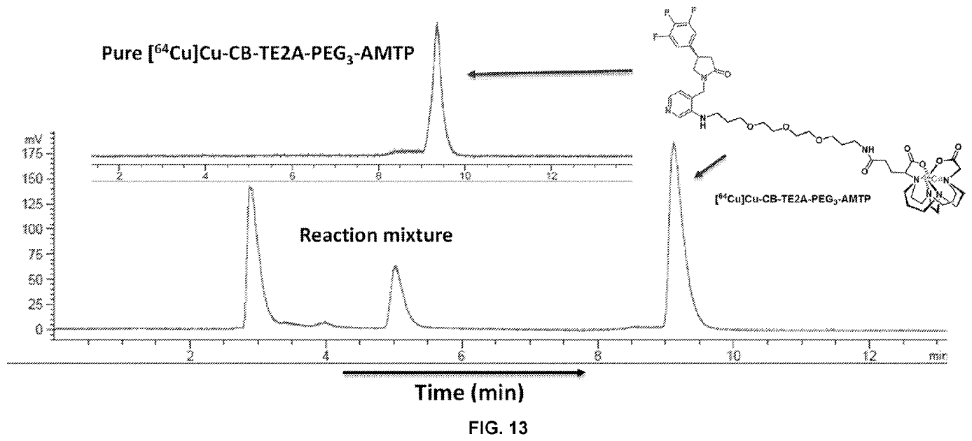

Preparation of [⁶⁴Cu]Cu-CB-TE2A-PEG₃-AMTP and [⁶⁴Cu]Cu-CB-TE2A-(PEG₃-AMTP)₂ by radiolabeling of CB-TE2A-PEG₃-AMTP and CB-TE2A-(PEG₃-AMTP)₂ with ⁶⁴Cu: For the synthesis of [⁶⁴Cu]Cu-CB-TE2A-PEG₃-AMTP, 12 μg of monovalent conjugate in 150 μL of 0.4 M NH₄OAc (pH=6.5) buffer and 17 mCi of ⁶⁴CuCl₂ were incubated at 75° C. for 0.5 h. To this reaction mixture, 5 μL of 5 mM EDTA solution was added and incubated for another 5 min (EDTA was used to remove non-specifically bound ⁶⁴Cu from the reaction mixture). The reaction mixture was diluted to 30 mL with milli-Q water and the purification of [⁶⁴Cu]Cu-CB-TE2A-PEG₃-AMTP was performed by passing the mixture through a Sep-Pak C-18 light cartridge. After rinsing the cartridge two times with 5 mL water, [⁶⁴Cu]Cu-CB-TE2A-PEG₃-AMTP (FIG. 11) was eluted with 1 mL ethanol. The product was analyzed by radio-HPLC to determine the radiochemical purity (>94%, FIG. 13). 9 mCi pure product was obtained at the end of synthesis with molar activity 736 mCi/μmol and non-decay corrected RCY ~53% (Table 1).

The partition coefficient log P for [⁶⁴Cu]Cu-CB-TE2A-PEG₃-AMTP was-0.49 (n=3), determined by the ratio of decay-corrected radioactivity concentrations in n-octanol and PBS. The in vitro stability of [⁶⁴Cu]Cu-CB-TE2A-PEG₃-AMTP was analyzed with human serum which showed ~7% decomposition at 24 h.

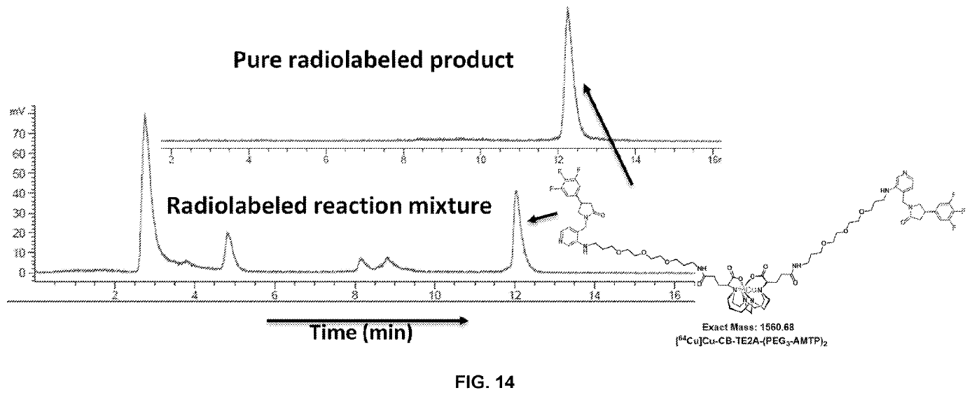

For the synthesis of [⁶⁴Cu]Cu-CB-TE2A-(PEG₃-AMTP)₂, 10 μg of CB-TE2A-(PEG₃-AMTP)₂ bivalent conjugate in 10 μL DMSO was added to 150 μL of 0.4 M NH₄OAc (pH=6.5) buffer containing 12 mCi of ⁶⁴CuCl₂ in 0.1 M HCl. The reaction mixture was incubated at 75° C. for 0.5 h. 5 μL of 5 mM EDTA was then added to the reaction mixture, which was allowed to incubate for another 5 min and followed the same procedure mentioned for monovalent conjugate. The reaction mixture was diluted to 30 mL (with milli-Q water) and the [⁶⁴Cu]Cu-CB-TE2A-(PEG₃-AMTP)₂ was purified by passing the mixture through a Sep-Pak tC-18 light cartridge. After rinsing the cartridge two times with 5 mL water, the ⁶⁴Cu-labeled product [⁶⁴Cu]Cu-CB-TE2A-(PEG₃-AMTP)₂ was eluted by pure 1 mL ethanol. The ⁶⁴Cu-labeled 4determine the radiochemical purity (>99%, FIG. 14). 3 mCi pure product was obtained at the end of synthesis with molar activity 467 mCi/umol and non-decay corrected RCY ~25% (Table 1). In comparison to [⁶⁴Cu]Cu-CB-TE2A-PEG₃-AMTP, the RCY for [⁶⁴Cu]Cu-CB-TE2A-(PEG₃-AMTP)₂ decreased by half, probably because of the generation of additional polar byproducts and intrinsic steric hindrance spurred due to the presence of two AMTP units. By altering the reaction temperature, initial activity of ⁶⁴Cu and stoichiometry of conjugates, it may be feasible to improve the RCY and molar activity further.

The partition coefficient log P for [⁶⁴Cu]Cu-CB-TE2A-(PEG₃-AMTP)₂ was 0.38 (n=3), determined by the ratio of decay-corrected radioactivity concentrations in n-octanol and PBS. The in vitro stability of [⁶⁴Cu]Cu-CB-TE2A-(PEG₃-AMTP)₂ was analyzed with human serum. For this purpose, [⁶⁴Cu]Cu-CB-TE2A-(PEG₃-AMTP)₂ (50 μCi) was mixed with 100 μL of human serum in a 5 mL quartz glass vial (n=3) and incubated at 37° C. for 1 and 24 h. 50 μL solution from each vial was diluted in 500 μL ethanol, centrifuged for 5 minutes (high speed centrifugation) and filtered out the supernatant by 0.2 μm filter. HPLC analysis of the supernatant solution showed no decomposition up to 24 h.

Measurement of Partition Coefficients: The partition coefficient (log P) of [⁶⁴Cu]Cu-CB-TE2A-PEG₃-AMTP and [⁶⁴Cu]Cu-CB-TE2A-(PEG₃-AMTP)₂ were-0.49 and 0.37 (n=3), respectively, measured by the ratio of decay-corrected radioactivity in n-octanol and PBS (Table 1). However, given that log P values range for the majority of drugs between-0.05 and 6.0, the optimal range should be 1.5-2.5 for neuroimaging agents, indicating that lipophilicity is necessary for BBB permeability.²⁶ The oncological SV2A agents for innervated cancers need to have a significantly distinct in vivo kinetics than their neuroimaging equivalents while preserving their excellent specificity and affinity for SV2A. The modified log P values of [⁶⁴Cu]Cu-CB-TE2A-PEG₃-AMTP and [⁶⁴Cu]Cu-CB-TE2A-(PEG₃-AMTP)₂ indicates its hydrophilic nature beneficial for SV2A specific oncological agents for innervated cancers.

TABLE 1
Radiosynthesis and physical properties of [⁶⁴Cu]Cu-CB-
TE2A-PEG₃-AMTP and [⁶⁴Cu]Cu-CB-TE2A-(PEG₃-AMTP)₂

| Conjugates | RCY | RCP | Specific Activity | logP |
|---|---|---|---|---|
| [⁶⁴Cu]Cu-CB-TE2A-PEG₃- | ~53% | >94% | 736′ mCi/μmol | −0.49 |
| AMTP | | | | |
| [⁶⁴Cu]Cu-CB-TE2A-(PEG₃- | ~25% | >99% | 467 mCi/μmol | 0.37 |
| AMTP)₂ | | | | |

* RCY: Radiochemical yield,
* RCP: Radiochemical purity

Cell Culture and Animal Models: The Institutional Animal Care and Use Committee (IACUC) at the University of Texas Southwestern Medical Center (Dallas, TX, USA) certified all animal experiments in this research. Tumor models were developed according to the animal protocol number APN 2017-102131 (approval date: 29 Dec. 2022; expiration date: 29 Dec. 2025). The mouse imaging experiments were carried out in accordance with APN 2020-102851 (approval date: May 26, 2020; expiration date: May 26, 2023). American Type Culture Collection (ATCC, Manassas, VA, USA, CRL-1435) supplied the neuroendocrine (TT, BON1, H727, H720, and H835) and prostate cancer (LNCaP, 22RV1, NCI-H660, PC3, and DU145) cells. The isogenic cell lines PC3_VC (SV2A^{high}) and PC3_sgPTP1B (SV2A^{low}) were produced by CRISPR-vector and CRISPR-PTP1B gene knockout, respectively. DU145_VC (SV2A^{high}) and DU145_sgPTP1B (SV2A^{low}) cells were also created in a similar manner. All cell lines have been cultured in RPMI medium supplemented with 10% fetal bovine serum, 1% penicillin/streptomycin, and 2 mM L-glutamine. The cells were harvested at 37° C. under 5% CO₂ environment. For tumor accumulation, tumor cells (1.0×10⁶) cells in 100 μL of phosphate buffered saline containing 30% Matrigel) were subcutaneously injected into the shoulders of severe combined immunodeficient (SCID) mice (NOD.CB17-Prkdcscid/NCrHsd, 6-8 weeks). The mice were given unfettered access to autoclaved water and commercial diets and were carefully monitored for tumor development and growth curve.

Cell Uptake Assay: The SV2A-selective cell uptake experiment was carried out using PC3-VC (SV2A^{high}), LnCap (SV2A^{low}) cells with monovalent [⁶⁴Cu]Cu-CB-TE2A-PEG₃-AMTP and bivalent [⁶⁴Cu]Cu-CB-TE2A-(PEG₃-AMTP)₂. Cells were placed in a 24-well plate coated through polylysine (Nalgene, Rochester, New York, USA) (~1.0×10⁶ cells per well, n=3) and kept inside a humidified incubator set at 37° C. with 5% CO₂ for 24 hours. The cells were subsequently incubated for 1 hour at room temperature with [⁶⁴Cu]Cu-CB-TE2A-PEG₃-AMTP or [⁶⁴Cu]Cu-CB-TE2A-(PEG₃-AMTP)₂ (~5.0×10⁵ CPM in each well) in 500 μL of binding buffer (20 mM tris, 150 mM NaCl, pH 7.4). After that, the solution was removed and the cells were meticulously washed three times with 500 μL of cold binding buffer before being solubilized with 500 μL of 1 M NaOH. A PerkinElmer 2480 gamma counter was used to count the radioactivity of the solutions (Richmond, CA, USA).

Internalization Assay: PC3-VC (SV2A^{high}) cells were used in the internalization assay. Nearly 3.0×10⁵ PC3-VC (SV2A^{high}) cells were seeded through each well of a 6-well plate and cultured for 24 hours in a humidified incubator at 37° C. with 5% CO₂. Upon gently rinsing the cells with the binding buffer (20 mM tris, 150 mM NaCl, pH 7.4), each well was treated with [⁶⁴Cu]Cu-CB-TE2A-PEG₃-AMTP or [⁶⁴Cu]Cu-CB-TE2A-(PEG₃-AMTP)₂ (~2×10⁵ CPM) diluted with 400 μL of the binding buffer. After incubating the cells for 3, 10, 30, 60, 90, 120, 150, and 180 minutes (n=3), they were adequately rinsed with cold binding buffer to extract the unbound [⁶⁴Cu]Cu-CB-TE2A-PEG₃-AMTP or [⁶⁴Cu]Cu-CB-TE2A-(PEG₃-AMTP)₂. The cells were then incubated with 0.5 mL of cold stripping buffer (150 mM NaCl, 50 mM glycine, pH 3.0) for 5 minutes to accumulate the radioconjugate from the cell surface. The stripping buffer was collected and then the cells were lysed in 500 μL of 1 M NaOH for 15 minutes at 37° C. to accumulate the internalized radioconjugate. The radioactivity of the solutions was determined with a PerkinElmer 2480 automatic gamma counter (Richmond, CA, USA). The triplicated data were represented as mean±standard deviation (s.d).

Small Animal PET/CT Imaging: The mice imaging experiments were carried out on a Mediso NanoScan PET/CT system (Mediso Medical Imaging Systems, Budapest, Hungary) outfitted with a 4-mouse bed. Tumor-bearing mice received anesthesia prior to each scan and were kept under 2% isoflurane during imaging. Each radiotracer (~3.5-4.5 MBq) was formulated in 150 UL of phosphate-buffered saline (PBS) and intravenously injected. A real-time dynamic (~0-60 minutes) and subsequent 20 minutes of static PET data acquisition were performed, followed by an anatomical reference 7 minutes CT scan. For quantitative imaging analysis, the regions of interest (ROIs) were precised manually. The uptake of [⁶⁸Ga]Ga-DOTA-PEG₃-AMTP, [⁶⁴Cu]Cu-CB-TE2A-PEG₃-AMTP or [⁶⁴Cu]Cu-CB-TE2A-(PEG₃-AMTP)₂ in ROIs was calculated as a percentage of injected dose per gram of tissue (% ID/g). PET/CT imaging of severe combined immunodeficiency (SCID) mice bearing H720 and BON1 tumor xenografts were evaluated with [⁶⁴Cu]Cu-CB-TE2A-PEG₃-AMTP for 0-60 min real-time dynamic PET scan. An additional 30 min PET scan was performed at 5 h p.i. (for H720) and 4 h p.i. (for BON1). PET/CT imaging with [⁶⁴Cu]Cu-CB-TE2A-(PEG₃-AMTP)₂ for SCID mice bearing SV2A^{high} H720 tumor xenografts were evaluated by 10-70 min real-time dynamic PET scan followed by 7 min CT. An additional 20 min PET scan was performed at 4.1 h p.i.

Western Blot Analysis: Cell homogenates were produced by lysing the cells on ice for 30 min utilizing freshly formulated buffer solution, which contained 150 mM NaCl, 50 mM Tris-HCl (pH 7.5), 0.1% Triton X-100, 2 mM phenylmethyl-sulfonyl fluoride, 1 mM sodium fluoride, 1 mM sodium orthovanadate, 1 mM sodium pyrophosphate, 10 mg/mL leupeptin, 10 mg/mL aprotinin and 1 mM EDTA. The cell lysates were centrifuged at 4° C. for 30 min at 14,000 rpm and the extracted proteins were loaded for sodium dodecyl sulfate-polyacrylamide gel electrophoresis (SDS-PAGE) using Bolt 4-12% NuPAGE gels (Life Technologies, Carlsbad, CA, USA). After that, the sample was blotted onto a nitrocellulose membrane using the Trans-Blot Turbo Transfer System (BIO-RAD, Hercules, CA, USA). After incubating the membrane at 4° C. for 15-20 hours in a solution of specific antibodies against PTP1B (ProteinTech, Rosemont, IL, USA) and SV2A (Abcam, Cambridge, UK), it was thoroughly cleaned and then incubated with horseradish-peroxidase-conjugated secondary antibodies for 2 hours. An Advansta ECL chemiluminescent detector (San Jose, CA, USA) was utilized to interpret the blotting outcomes. Actin (Santa Cruz Biotechnology, Dallas, TX, USA) was used as a loading control.

Immunohistochemistry (IHC): Tumors were excised after the experiments and immediately fixed in 10% neutral-buffered formalin for 48 h. Tissue samples were processed before being embedded in paraffin blocks. The paraffined slides were de-paraffinized, rehydrated and the antigen was retrieved using citrate buffer. Slides were blocked by intelliPATH™ Background Punisher (IP974G20, BioCare Medical, USA), followed by peroxidase inhibitor (IPB5000G20, BioCare Medical, USA) and mouse antigen killer (RBM961G, BioCare Medical, USA). The slides were stained by anti-SV2A antibody (HPA007863, Sigma, USA) for 1 hr at room temperature and stained with a universal secondary antibody conjugated with HRP (M2U522G, BioCare Medical, USA). The SVA2 expression was visualized by DAB staining with hematoxylin to stain the cytosol and nucleus. The representative photograph was taken with a Nikon microscope.

Human Serum Stability of [⁶⁴Cu]Cu-CB-TE2A-PEG₃-AMTP and [⁶⁴Cu]Cu-CB-TE2A-(PEG₃-AMTP)₂: The in vitro stability of [⁶⁴Cu]Cu-CB-TE2A-PEG₃-AMTP and [⁶⁴Cu]Cu-CB-TE2A-(PEG₃-AMTP)₂ were analyzed with human serum. For this purpose, 50 μCi of each radiotracer was added with 100 μL of human serum in a 5 mL quartz glass vial (n=3) and incubated at 37° C. for 1 and 24 h. From each vial, 50 μL solution was diluted in 500 μL ethanol, centrifuged for 5 minutes (high-speed centrifugation) and filtered out the supernatant with a 0.2 μm filter. HPLC analysis of the supernatant solution showed ~7% decomposition for [⁶⁴Cu]Cu-CB-TE2A-PEG₃-AMTP and no decomposition for [⁶⁴Cu]Cu-CB-TE2A-(PEG₃-AMTP)₂ at 24 h. The prolonged human serum stability of both radioconjugates could promote the innervated cancer imaging and treatment results by reducing the metabolite formation during in vivo circulation.

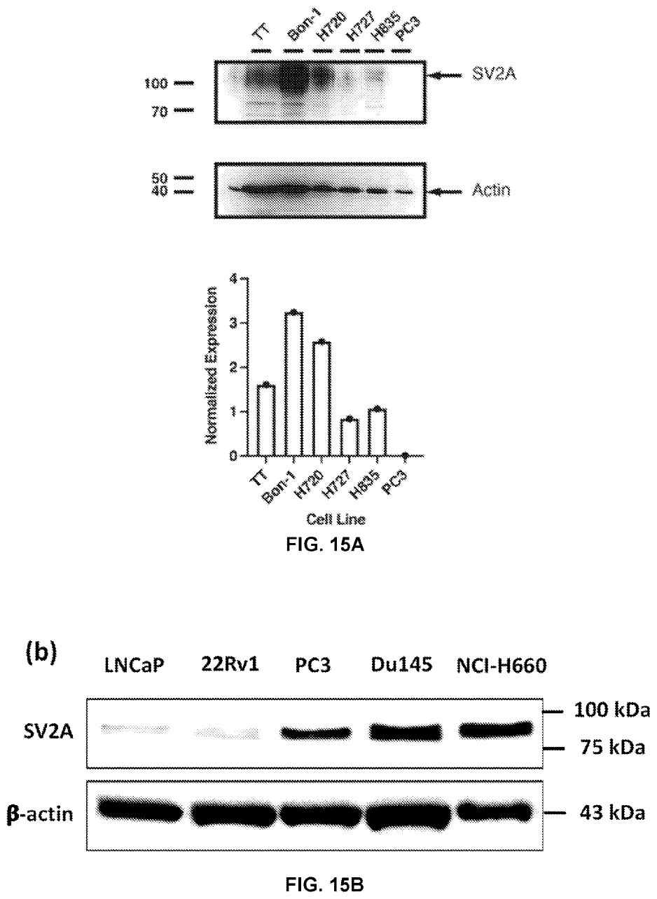

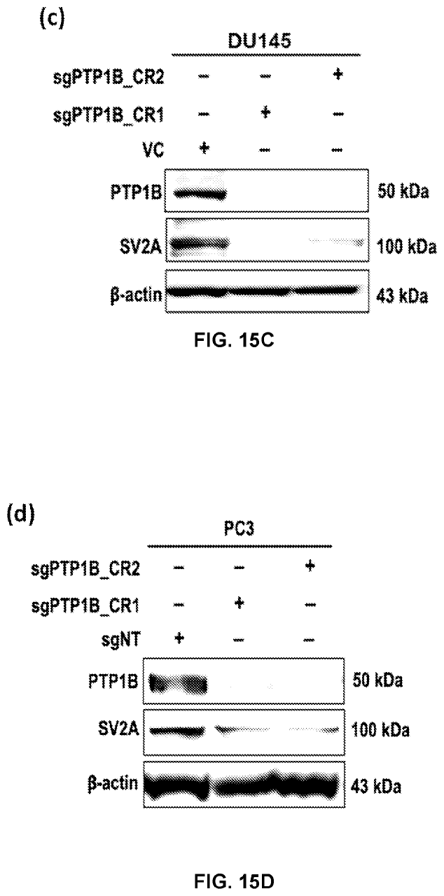

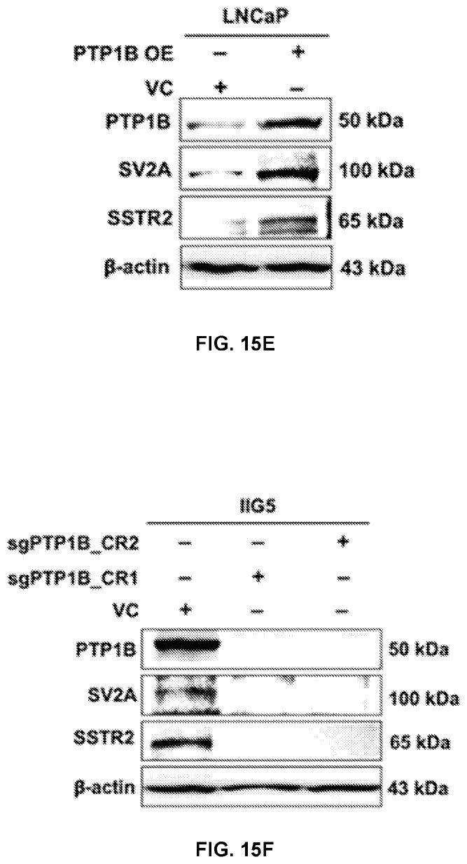

SV2A Protein Expression in Cell Lines: It has been established that several neuroendocrine prostate cancer (NEPC) cell lines (DU145, PC-3, NCI-H660) exhibited significantly elevated SV2A expressions than non-NEPC cell lines (LNCaP, 22RV1).²² To further investigate the prospective relevance of SV2A as a target biomarker for innervated cancer, an SV2A protein expression assay using several neuroendocrine lung cancer cell lines, including TT, BON1, H727, H720, and H835 was conducted. Distinctly higher SV2A expressions in TT, BON1, and H720 and low SV2A expressions in H727 and H835 cells were identified (FIGS. 15A-D). On the other hand, CRISPR-PTP1B gene knockout DU145_sgPTP1B (CR1 and CR2) and PC3_sgPTP1B (CR1 and CR2) showed a significant reduction of SV2A expressions compared to the DU145-VC and PC3-VC, generated by CRISPR-vector from parental DU145 and PC3 cells (FIGS. 15A-D). Most of the selected cell lines displayed higher levels of SV2A expression, which implies that SV2A would be a valuable biomarker for innervated cancer theranostics.

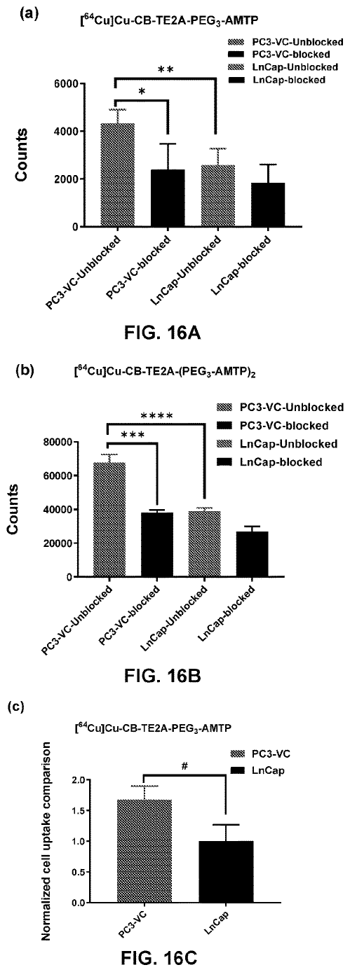

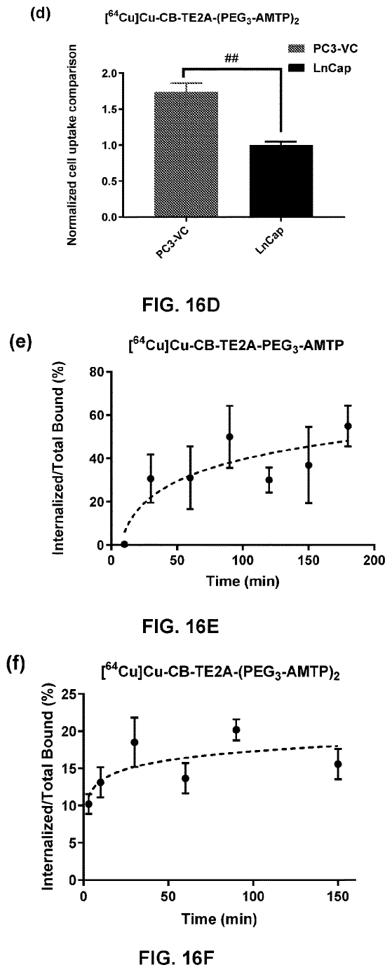

SV2A-specific Binding Assays: The incorporation of the PEG₃ linker and CB-TE2A chelator in the embedded molecular framework was intended to preserve the desired SV2A-selective cell binding and SV2A-mediated cell internalization with modified lipophilicity. The SV2A specific uptake of [⁶⁴Cu]Cu-CB-TE2A-PEG₃-AMTP and [⁶⁴Cu]Cu-CB-TE2A-(PEG₃-AMTP)₂ was measured in PC3-VC (SV2A^{high}) and LnCap (SV2A^{low}) cells. As shown in FIGS. 16A-D, the uptake of [⁶⁴Cu]Cu-CB-TE2A-PEG₃-AMTP and [⁶⁴Cu]Cu-CB-TE2A-(PEG₃-AMTP)₂ demonstrated a 1.68 and 1.74 times higher accumulation in PC3-VC cells than that in LnCap cells after 1 h of incubation, confirming the desired SV2A-selective binding of the conjugates. The SV2A binding blockade of 1 mM of SynVesT-1, resulting in a 1.80 and 1.77 times reduction in SV2A-binding of [⁶⁴Cu]Cu-CB-TE2A-PEG₃-AMTP and [⁶⁴Cu]Cu-CB-TE2A-(PEG₃-AMTP)₂ respectively in PC3-VC cells. Furthermore, time-dependent incubation with PC3-VC cells showed that approximately 40% of [⁶⁴Cu]Cu-CB-TE2A-PEG₃-AMTP and 15% of [⁶⁴Cu]Cu-CB-TE2A-(PEG₃-AMTP)₂ were internalized into the cells after 160 min (FIGS. 16E-F). Although the binding intensity paved the way toward further in vivo evaluation for both the radioconjugates, additional linker modification to modify the lipophilicity could enhance the cell binding and internalization measures.

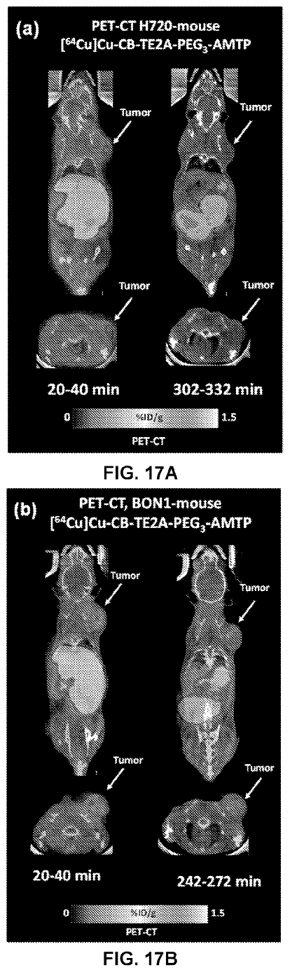

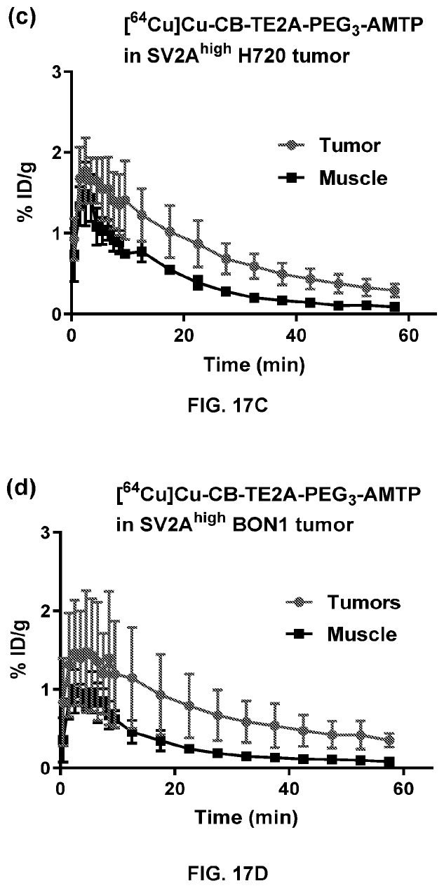

Comparative Imaging Studies of the Two Labeled Conjugates in Various Mouse Models: In vivo evaluation of monovalent [⁶⁴Cu]Cu-CB-TE2A-PEG₃-AMTP: Although SV2A was validated as a promising biomarker for PET imaging of NEPC tumors with ¹⁸F-SynVesT-1,²² here, the metal radionuclide-based theranostics conjugates were widely investigated for in vivo physiognomies in various innervated tumor types. Encouraged with the SV2A protein expression assay in several cell lines, H720 and BON1 (high-SV2A expressing) lung cancer cell lines were elected to develop severe combined immunodeficiency (SCID) mice tumor xenografts and further imaged with [⁶⁴Cu]Cu-CB-TE2A-PEG₃-AMTP. The PET/CT images showed that both H720 and BON1 tumors were clearly observable with distinguishable tumor contrast at 20-40 min time duration (FIGS. 17a and 17b). Quantitative uptake analysis (FIGS. 17c and 17d) showed that the conjugate [⁶⁴Cu]Cu-CB-TE2A-PEG₃-AMTP has reasonably higher tumor uptake (1.02±0.32% ID/g for H720 and 0.93±0.34% ID/g for BON1) then muscle at 20 min p.i. with a tumor to muscle ratios of 1.85 and 2.68 respectively.

PET/CT imaging of SCID mice bearing SV2A⁺ H720 and BON1 (high-SV2A expressing) tumor xenografts were evaluated by 0-60 min real-time dynamic PET scan followed by 7 min CT. An additional 30 min PET scan was performed at 5 h P.I. for SV2A⁺ H720 and 4 h P.I for SV2A⁺ BON1 tumor xenografts. The PET/CT images showed that SV2A⁺ H720 and BON1 tumors were observable with distinguishable tumor contrast at 10-40 min time duration (FIG. 17A and FIG. 17B). Quantitative uptake analysis (FIG. 17C and FIG. 17D) showed that [⁶⁴Cu]Cu-CB-TE2A-PEG₃-AMTP has moderately higher tumor uptake than muscle at all-time points throughout the scans. The larger and hydrophilic molecular construction reduces the BBB penetration and showed minimal brain uptake in dynamic time points.

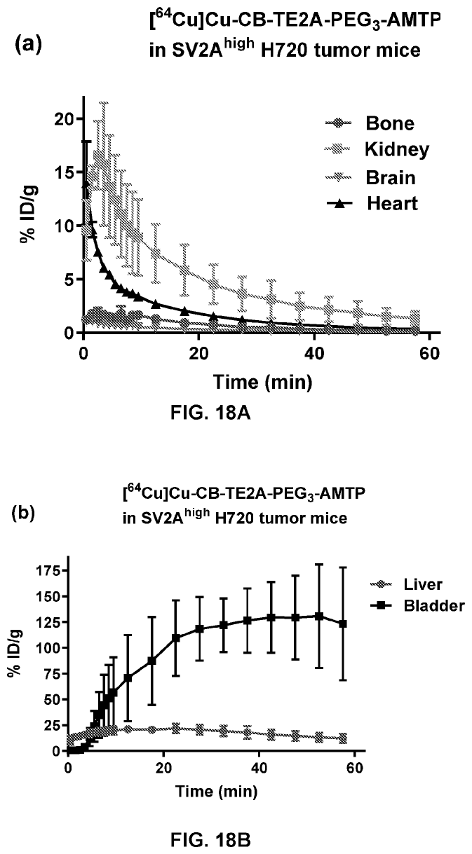

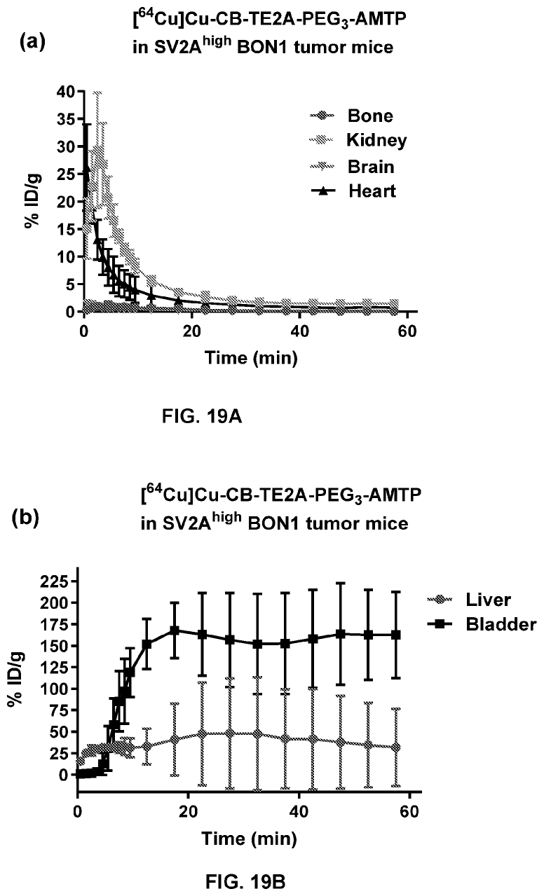

Despite the PET imaging of ¹⁸F-SynVesT-1 was executed with NCI-H660 tumor xenografts, monovalent [⁶⁴Cu]Cu-CB-TE2A-PEG₃-AMTP showed relatively superior tumor uptake and tumor-to-muscle ratio at earlier time points in H720 and BON1 tumor mice. This behavior could be attributed to the lipophilic modification by regulating the log P value from 2.32 (¹⁸F-SynVesT-1)²² to −0.49 ([⁶⁴Cu]Cu-CB-TE2A-PEG₃-AMTP). The macromolecular design (exact mass 981.41) and elevated hydrophilicity of [⁶⁴Cu]Cu-CB-TE2A-PEG₃-AMTP contributed to the reduction of BBB permeability and demonstrated a noteworthy decline in brain uptake of 0.35±0.04% ID/g for H720 and 0.30±0.11% ID/g for BON1 tumor mice at 20 min p.i. (FIG. 18a and FIG. 19a). The substantially lower brain uptake of [⁶⁴Cu]Cu-CB-TE2A-PEG₃-AMTP than ¹⁸F-SynVesT-1²² and ¹¹C/¹⁸F-UCB-J²⁷ allied with our repurposing strategy of targeting SV2A for innervated cancer oncology rather than neurological dysfunction. Both the tumor xenografts demonstrated rapid radiotracer excretion via kidneys and an increasing trend of bladder accumulation, indicating significant renal clearance of hydrophilic [⁶⁴Cu]Cu-CB-TE2A-PEG₃-AMTP (FIGS. 18 and 19). On the other hand, the significant abdominal accumulation indicates the necessity of further structural optimization to upgrade in vivo kinetics and decrease the generation of lipophilic liver metabolites.

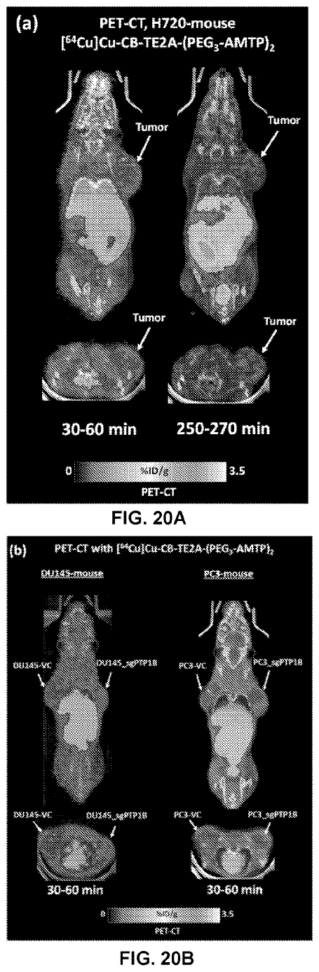

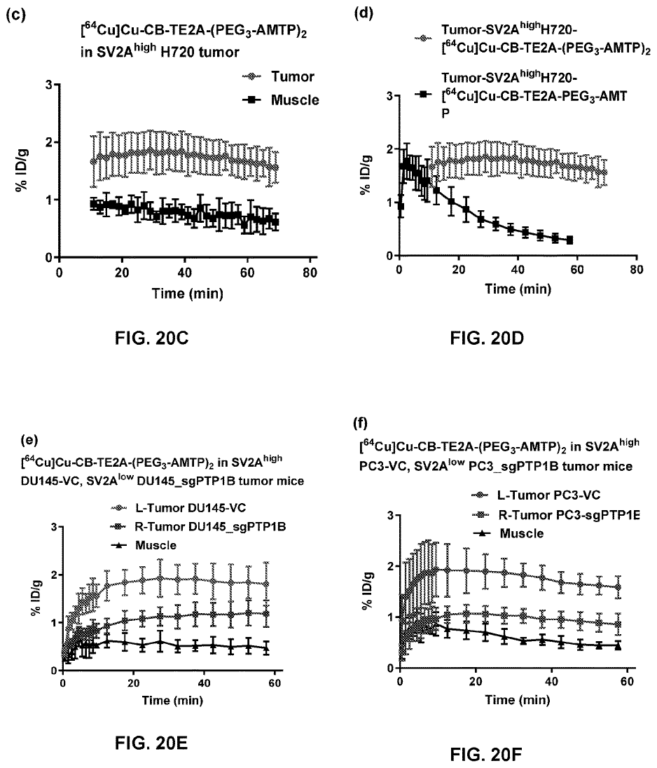

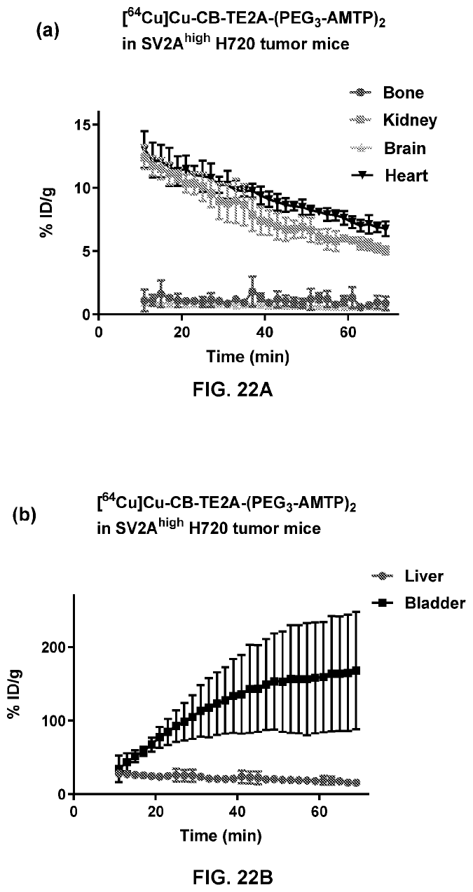

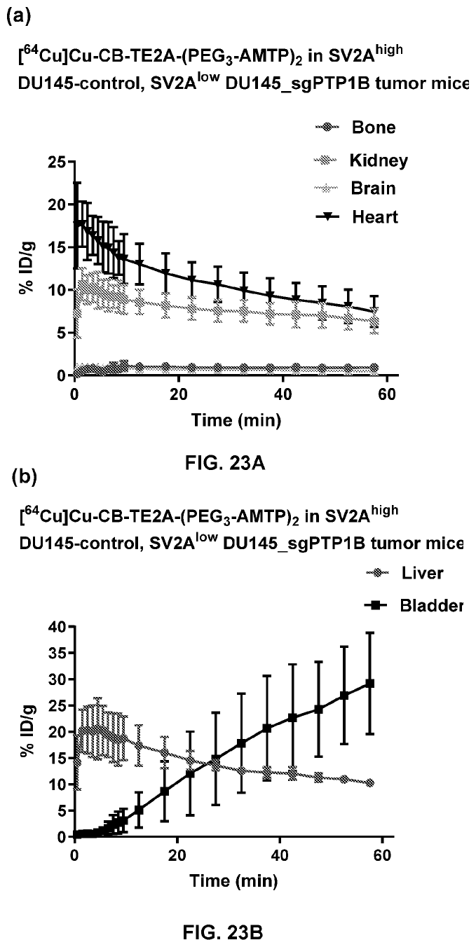

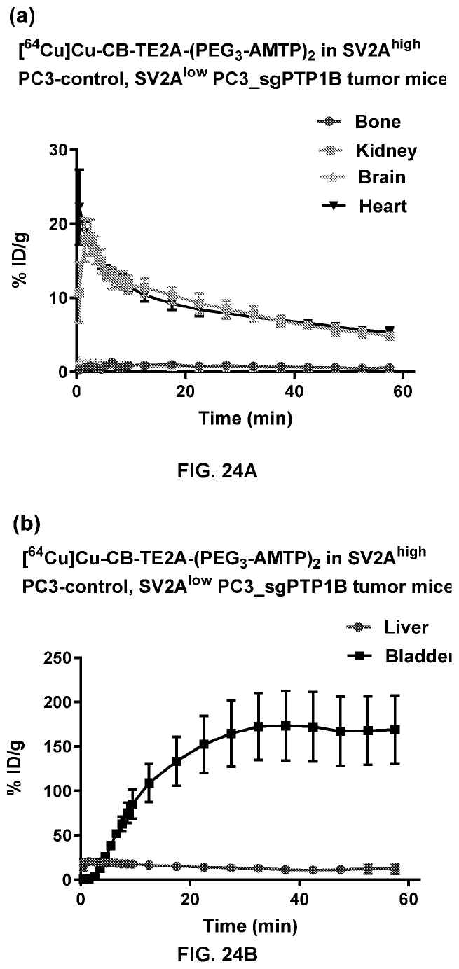

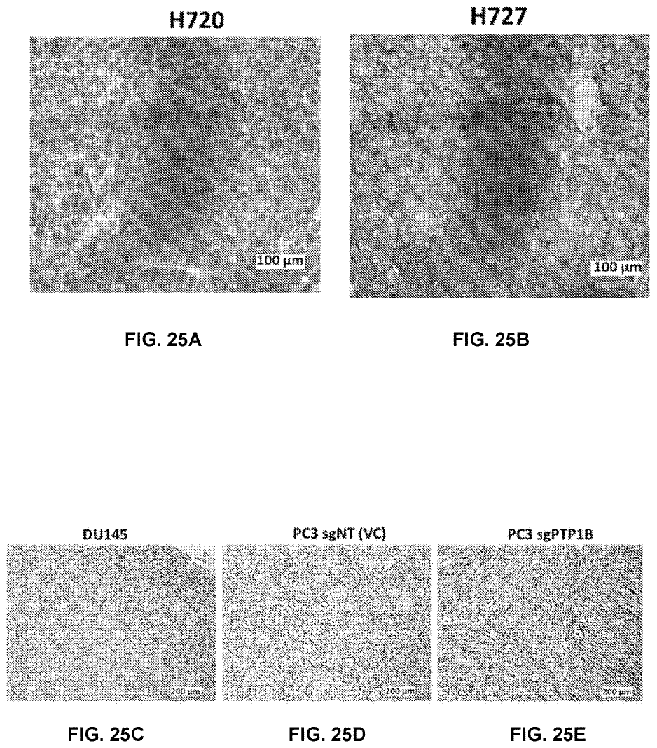

In vivo evaluation of bivalent [⁶⁴Cu]Cu-CB-TE2A-(PEG₃-AMTP)₂: Rapid tumor clearance of the monovalent conjugate (~80% in 50 minutes) limits its utility for the repurposing strategy concerning inefficient radiotherapy. The bivalent conjugate [⁶⁴Cu]Cu-CB-TE2A-(PEG₃-AMTP)₂ with two SV2A targeting cores could elongate tumor retention to enhance the therapeutic output. The SV2A selective tumor retention of [⁶⁴Cu]Cu-CB-TE2A-(PEG₃-AMTP)₂ was evaluated in SCID mice bearing the SV2A^{high} (H720, H727, DU145-VC, PC3-VC) and SV2A^{low} (DU145_sgPTP1B, PC3_sgPTP1B) tumor xenografts. The PET/CT images showed that SV2A^{high} H720 tumors were clearly observable (FIG. 20A) with relatively higher tumor uptake (1.7±1.4% ID/g) than muscle (0.8±1.2% ID/g) at 40 min p.i. and tumor to muscle ratio was 2.33. Interestingly, the tumor uptake was retained in a similar range at later time points (e.g., 1.56±0.26% ID/g at 70 min p.i. with tumor to muscle ratio of 2.55). The multivalence approach for the radioconjugate uplifted the SV2A binding affinity and extended the tumor retention at later time points, crucial for radionuclide therapy. The lower brain uptake of 0.76±0.11% ID/g at 40 min p.i. was supported by the macromolecular structure (MW 1559.68) and low lipophilicity (log P: 0.37) of [⁶⁴Cu]Cu-CB-TE2A-(PEG₃-AMTP)₂, which reduces the BBB penetration (FIG. 22A). Additionally, the renal clearance of [⁶⁴Cu]Cu-CB-TE2A-(PEG₃-AMTP)₂ (FIGS. 22A and 22B) was observed at a moderate rate compared to monovalent conjugate due to the lipophilic modification.

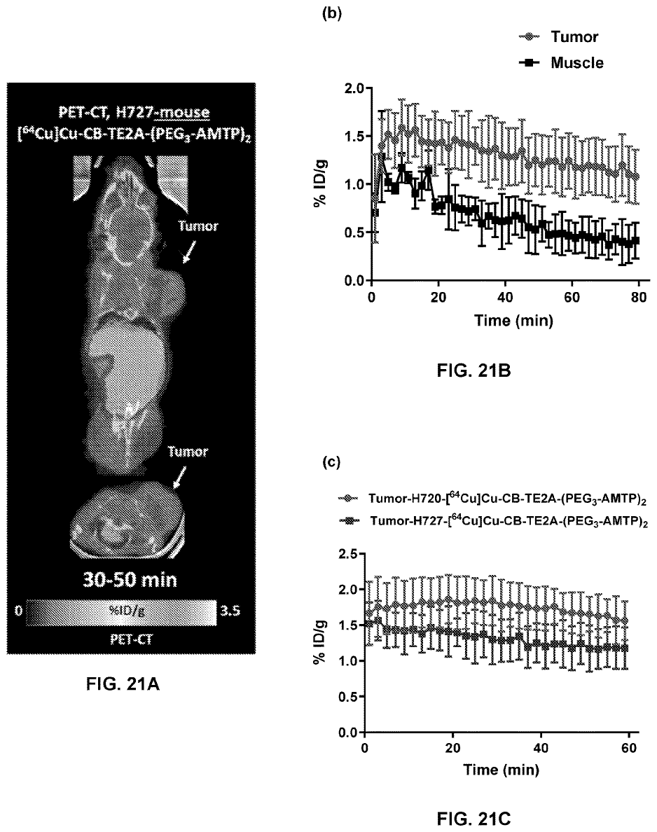

In a head-to-head comparison of the quantitative uptake analysis for [⁶⁴Cu]Cu-CB-TE2A-(PEG₃-AMTP)₂ and [⁶⁴Cu]Cu-CB-TE2A-PEG₃-AMTP in H720 tumors (FIG. 20D) showed that the bivalent radioconjugate has significantly higher tumor retention throughout the scans. The bivalent to monovalent tumor uptake ratio was 3.72 at 40 min p.i., which imply the significance of multivalent structural modification. PET/CT imaging of SCID mice bearing SV2A medium H727 tumor xenografts showed reasonable uptake of 1.29±0.34% ID/g at 20 min p.i. with a tumor to muscle ratios of 2.11 (FIGS. 21A and 21B). A head-to-head comparison of the quantitative uptake analysis (FIG. 21C) showed that the conjugate [⁶⁴Cu]Cu-CB-TE2A-(PEG₃-AMTP)₂ has relatively higher tumor retention in H720 than H727 tumor, which validates the results of the SV2A protein expression assay.

Additional PET/CT imaging of SCID mice bearing SV2A^{high} DU145-VC (left shoulder) and SV2A^{low} DU145_sgPTP1B (right shoulder) dual tumor xenografts were evaluated to know the SV2A selectivity of [⁶⁴Cu]Cu-CB-TE2A-(PEG₃-AMTP)₂. The PET/CT images showed that Du145-VC tumors were clearly observable with significant tumor contrast at 30-60 min then DU145_sgPTP1B indicating SV2A selective tumor uptake (FIG. 20B, left). [⁶⁴Cu]Cu-CB-TE2A-(PEG₃-AMTP)₂ showed relatively high accumulation in DU145-VC tumor (1.91±0.31% ID/g) than DU145_sgPTP1B tumor (1.17±0.25% ID/g) at 40 min p.i (FIG. 20E). PET/CT imaging of SCID mice bearing SV2A^{high} PC3-VC (left shoulder) and SV2A^{low} PC3_sgPTP1B tumor (right shoulder) dual tumor xenografts likewise exhibited SV2A selective tumor uptake of the radioconjugate (FIG. 20B, right). Quantitative uptake analysis (FIG. 20F) showed that the conjugate [⁶⁴Cu]Cu-CB-TE2A-(PEG₃-AMTP)₂ has 1.86 times higher uptake in PC3-VC compare to PC3_sgPTP1B at 40 min p.i. Lower brain uptake of [⁶⁴Cu]Cu-CB-TE2A-(PEG₃-AMTP)₂ was observed in both the dial tumor models due to the higher molecular weight and reduced lipophilicity. The IHC staining of SV2A on the bisected tumors demonstrated the localization of the SV2A protein in tumor microenvironment. The expression SV2A in DU145 tumor was observed in both cytosol and the cell membrane while the expression was mostly in the cell membrane in H720 and H727. The optical density of the DAB staining fit the trend of the trace uptake in the tumors.

The organ uptake and tumor targeting potency of [⁶⁴Cu]Cu-CB-TE2A-(PEG₃-AMTP)₂ was evaluated in severe combined immunodeficiency (SCID) mice bearing the SV2A⁺ H720, H727, DU145-control, PC3-control and SV2A DU145_sgPTP1B, PC3_sgPTP1B subcutaneous tumor xenografts. [⁶⁴Cu]Cu-CB-TE2A-(PEG₃-AMTP)₂ (~80 μCi, in 120 μL) was intravenously injected via the tail vein in tumor-bearing SCID mice for small animal PET/CT imaging. Scans were executed with a Mediso PET/CT multimodality system and the mouse was sedated with 1.8% isoflurane anesthesia (100% O₂ as carrier) throughout the acquisition. Real-time dynamic PET scans were conducted for suitable image contrast followed by a 7 min CT acquisition. All the PET and CT data were reconstructed, and regions of interest (ROIs) were marked as displayed by CT to quantify the tracer uptake as percent injected dose per gram of tissue (% ID/g).

PET/CT imaging of SCID mice bearing SV2A⁺ H720 tumor xenografts were evaluated by 10-70 min real-time dynamic PET scan followed by 7 min CT. An additional 20 min PET scan was performed at 4 h p.i. The PET/CT images showed that SV2A positive H720 tumors were clearly observable with maximum tumor uptake at 30-50 min time duration (FIG. 20A). Quantitative uptake analysis (FIG. 20C) showed that [⁶⁴Cu]Cu-CB-TE2A-(PEG₃-AMTP)₂ has relatively higher tumor uptake (1.7±1.4% ID/g) than muscle (0.8±1.2% ID/g) at 40 min p.i. The larger and hydrophilic molecular construction (MW 1559.68) reduces the BBB penetration and showed minimal brain uptake. Comparison of the quantitative uptake analyses for [⁶⁴Cu]Cu-CB-TE2A-(PEG₃-AMTP)₂ and [⁶⁴Cu]Cu-CB-TE2A-PEG₃-AMTP in SV2A⁺ H720 tumors (FIG. 20D) showed that [⁶⁴Cu]Cu-CB-TE2A-(PEG₃-AMTP)₂ has significantly higher tumor contrast and tumor retention, which implies the value of multivalent structural modification.

PET/CT imaging of SCID mice bearing SV2A⁺ H727 expressing tumor xenografts were evaluated by 0-80 min real-time dynamic PET scan followed by 7 min CT. The PET/CT imaging data showed that SV2A⁺ H727 tumors had moderate uptake at 30-50 min time duration (FIG. 21A) compared to H720 tumors. Quantitative uptake analysis (FIG. 21B) showed that [⁶⁴Cu]Cu-CB-TE2A-(PEG₃-AMTP)₂ has higher tumor uptake than muscle area. The larger and hydrophilic molecular construction reduces the BBB penetration and showed minimal brain uptake in dynamic time points. Comparison of the quantitative uptake analyses in SV2A⁺ H720 and SV2A⁺ H727 tumors (FIG. 21C) showed that [⁶⁴Cu]Cu-CB-TE2A-(PEG₃-AMTP)₂ has higher tumor retention in SV2A⁺ H720, which suggests that SV2A⁺ H720 has higher SV2A expression than SV2A⁺ H727 tumors, which is in agreement with the IHC analysis.

PET/CT imaging of SCID mice bearing SV2A⁺ DU145-control (left shoulder) and SV2A-DU145_sgPTP1B (right shoulder) tumor xenografts were evaluated by 0-60 min real-time dynamic PET scan followed by 7 min CT. The PET/CT imaging data showed that SV2A⁺ Du145-control tumors were clearly observable with maximum tumor uptake at 30-60 min time duration (FIG. 20B). Quantitative uptake analysis (FIG. 20E) showed that [⁶⁴Cu]Cu-CB-TE2A-(PEG₃-AMTP)₂ has relatively higher SV2A⁺ DU145-control tumor uptake than muscle and SV2A-DU145_sgPTP1B tumor at 25 min P.I. PET/CT imaging of SCID mice bearing SV2A⁺ PC3-control (left shoulder) and SV2A-PC3_sgPTP1B tumor (right shoulder) tumor xenografts were evaluated by 0-60 min real-time dynamic PET scan followed by 7 min CT. The PET/CT imaging data showed that SV2A⁺ PC3-control tumors were clearly observable with maximum tumor uptake at 30-60 min time duration. Quantitative uptake analysis (FIG. 20F) showed that [⁶⁴Cu]Cu-CB-TE2A-(PEG₃-AMTP)₂ has relatively higher SV2A⁺ PC3-control tumor uptake than muscle and SV2A-PC3_sgPTP1B tumor at 10 min p.i. Lower brain uptake represents the reduced BBB penetration due to the larger molecular construction.

Recent findings have improved our understanding of how diseases, including cancer, can affect normal neuron development and disrupt communication within the nervous system. Although there were early reports in the 20^{th} century of nerves invading tumors, it had been widely accepted that these tumor microenvironment (TME) elements were silent bystanders. Research on tumor innervation has lately attracted attention since cancer cells, on purpose, interact intimately with the nerves that penetrate the tumor. Instead of being a passive component of the TME, nerves play a functional role in cancer formation and cancer cells specifically target those nerves to take advantage of their impact on expansion. As evidenced by several seminal reports, the nervous system plays a decisive part in the onset and advancement of cancer.²⁸ Ablation of particular nerve subtypes (sympathetic/parasympathetic) inhibits the expansion of tumors significantly.

Here, radiotheranostics were developed for innervated cancer oncology by leveraging the vesical protein targeting efficacy of neuroimaging agents. Our pilot study with ¹⁸F-SynVesT-1 showed substantial levels of absorption and retention in the brain, liver, and intestines because of its eminent lipophilicity, necessary for a neuroimaging agent to pass BBB. Notably, the unavoidable high concentration of SV2A in the brain might render neuro-oncological imaging of SV2A challenging with ¹⁸F-SynVesT-1 due to the high brain uptake. A sizable amount of renal excretion of ¹⁸F-activity was recognized, likely due to the less lipophilic ¹⁸F-SynVesT-1 metabolites. Therefore, although the proof-of-concept data was promising, further structural optimization was required for oncological benefits. To decrease extensive brain exposure, the basic molecular features of a radiotracer responsible for its passive transfer across the BBB were eliminated. In general, low molecular weight (<500 Da), small cross-sectional area (<80 Å²), poor hydrogen bonding capability, and absence of formal charge enhance passive transfer.^{29,30} Lipophilicity is an essential element influencing passive brain entrance determined by Log P.³⁰ Given the enhanced lipophilicity of the radiotracer, we could anticipate an increase in passive entrance into the brain. Our represented molecular design for innervated cancer theranostics includes increased hydrophilicity with modified Log P, high plasma free fraction (fp) and high molecular weight (>500 Da).

To retain a chemically identical structural construct during imaging and therapy, we chose metal-radionuclide (^{64/67}Cu) instead of radioactive nucleophiles (¹⁸F, ¹¹C, etc.) to retain analogous in vivo kinetics. The chelating ligand CB-TE2A comprises optimal metal chelation for superior in vivo stability. Taking advantage of the multivalent strategy, the representative SV2A targeting conjugates stretched to have bivalent SV2A targeting (AMTP) ligands to augment the diagnosis and therapeutic efficacy. The SV2A targeting AMTP ligand was conjugated with CB-TE2A through a polyethylene glycol (PEG₃) linker to prepare monovalent CB-TE2A-PEG₃-AMTP and bivalent CB-TE2A-(PEG₃-AMTP)₂ conjugate in a modular synthesis strategy that is readily adaptable for various tracer development. The reduced radiochemical yield for [⁶⁴Cu]Cu-CB-TE2A-(PEG₃-AMTP)₂ (~25% RCY) in comparison to [⁶⁴Cu]Cu-CB-TE2A-PEG₃-AMTP (~53% RCY) may be attributed to the intrinsic steric congestion in the bivalent conjugate. Small animal PET imaging in mice bearing H720 xenografts showed the tumor uptake of [⁶⁴Cu]Cu-CB-TE2A-PEG₃-AMTP peaked at 10 min p.i. (1.40±0.49% ID/g) with a tumor to muscle ratio of 1.88 followed by a rapid clearance (~80% clearance in 50 minutes). The monovalent nature of [⁶⁴Cu]Cu-CB-TE2A-PEG₃-AMTP with higher hydrophilicity (log P: −0.49) could be responsible for rapid tumor clearance. In contrast, a gradually increasing profile was observed for [⁶⁴Cu]Cu-CB-TE2A-(PEG₃-AMTP)₂ until 40 min p.i (1.84±0.34% ID/g) with higher tumor to muscle ratio of 2.33 and extended retention up to 70 min p.i. Furthermore, the comparative time-activity curves exhibited that the bivalent [⁶⁴Cu]Cu-CB-TE2A-(PEG₃-AMTP)₂ maintained significantly higher tumor uptake and retention than its monovalent counterpart throughout the scans. The tumor accumulation and tumor to muscle ratios for [⁶⁴Cu]Cu-CB-TE2A-(PEG₃-AMTP)₂ were noticeably higher than [⁶⁴Cu]Cu-CB-TE2A-PEG₃-AMTP and ¹⁸F-SynVesT-1 probably due to the bivalent nature and appropriate log P value of 0.37.

The represented multivalent effect for amplified ligand-receptor interactions and the lipophilic metamorphosis of [⁶⁴Cu]Cu-CB-TE2A-(PEG₃-AMTP)₂ was further assessed in SCID mice bearing dual tumor model (model 1: SV2A^{high} DU145-VC, SV2A^{low} DU145_sgPTP1B tumor and model 2: SV2A^{high} PC3-VC, SV2A^{low} PC3_sgPTP1B tumor) which displayed the SV2A selective tracer accumulation with extended retention. The tumor to muscle ratios were 3.83 and 3.57 for model 1 (SV2A^{high} DU145-VC vs. muscle) and model 2 (SV2A^{high} PC3-VC vs. muscle) at 60 min p.i. Additionally, both the conjugates showed significantly low brain accumulation to benefit the neurooncology outside brain with low exposure in the brain. PET imaging demonstrated rapid radiotracer accumulation/excretion via kidneys and an increasing trend of bladder accumulation indicating renal excretion. Overall the tumor uptake and retention data of bivalent [⁶⁴Cu]Cu-CB-TE2A-(PEG₃-AMTP)₂ created a strong fundamental platform for innervated cancer theranostic and warrants further preclinical radiotherapeutic efficacy study for translational purposes.

### Example 2: Preparation of DOTA-PEG₃-AMTP

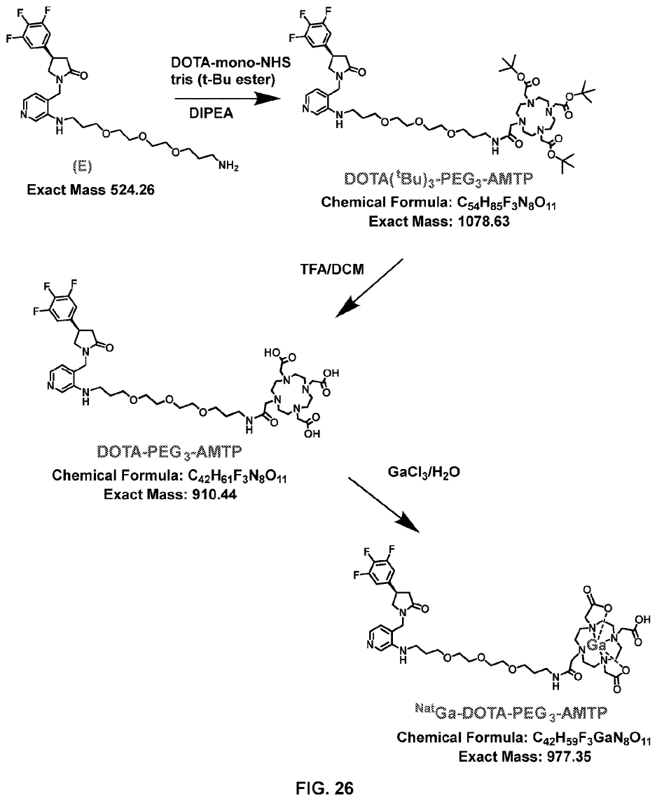

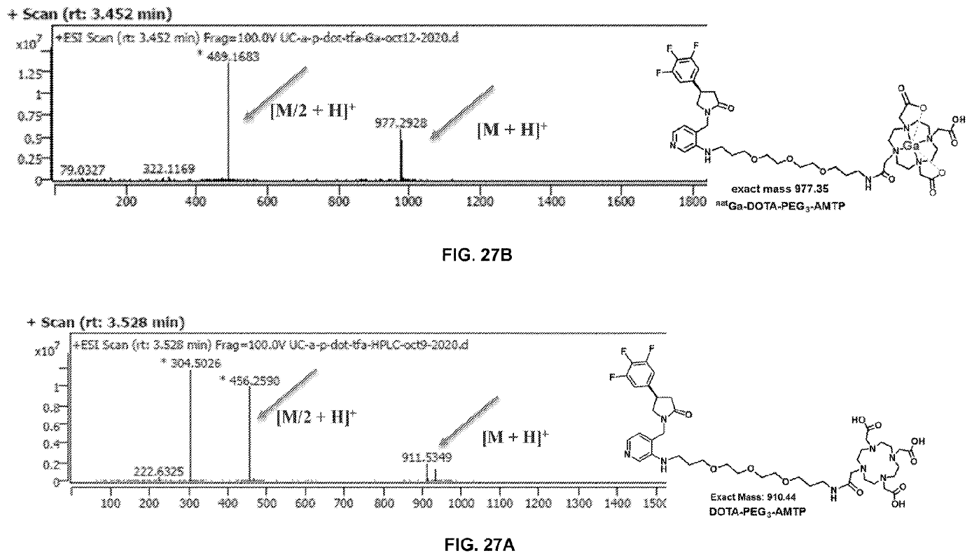

The preparation of DOTA-PEG₃-AMTP precursor was accomplished as shown in FIG. 26. The preparation of compound E is described in more detail above in Example 1. Compound E was reacted with DOTA-mono-NHS tris (t-Bu ester) under basic (DIPEA) condition to produce DOTA-PEG₃-AMTP-3′Bu. DOTA (^{t}Bu)₃-PEG₃-AMTP was reacted with 90% trifluoroacetic acid in dichloromethane yielding DOTA-PEG₃-AMTP. ^{nat}Ga chelated standard conjugate prepared by reacting DOTA-PEG₃-AMTP with aqueous GaCl₃ solution. Characterization of the conjugates by LC-MS is shown in FIGS. 27A and 27B.

DOTA (^{t}Bu)₃-PEG₃-AMTP
MS (ESI) m/z calcd for C₅₄H₈₅F₃N₈O₁₁: 1078.63; found: 1079.62 ([M+H]⁺, 540.31 [M/2+H]⁺), 360.54 [M/3+H]⁺).

### DOTA-PEG₃-AMTP

MS (ESI) m/z calcd for C₄₂H₆₁F₃N₈O₁₁: 910.44; found: 911.53 [M+H]⁺, 456.25 [M/2+H]⁺, 304.50 [M/3+H]⁺.
   ^{nat}Ga chelated standard of DOTA-PEG₃-AMTP
MS (ESI) m/z calcd for C₄₂H₅₉F₃GaN₈O₁₁: 977.35; found: 977.29 [M+H]⁺, 489.16 [M/2+H]⁺.

### Example 3: Preparation of [⁶⁸Ga]Ga-DOTA-PEG₃-AMTP

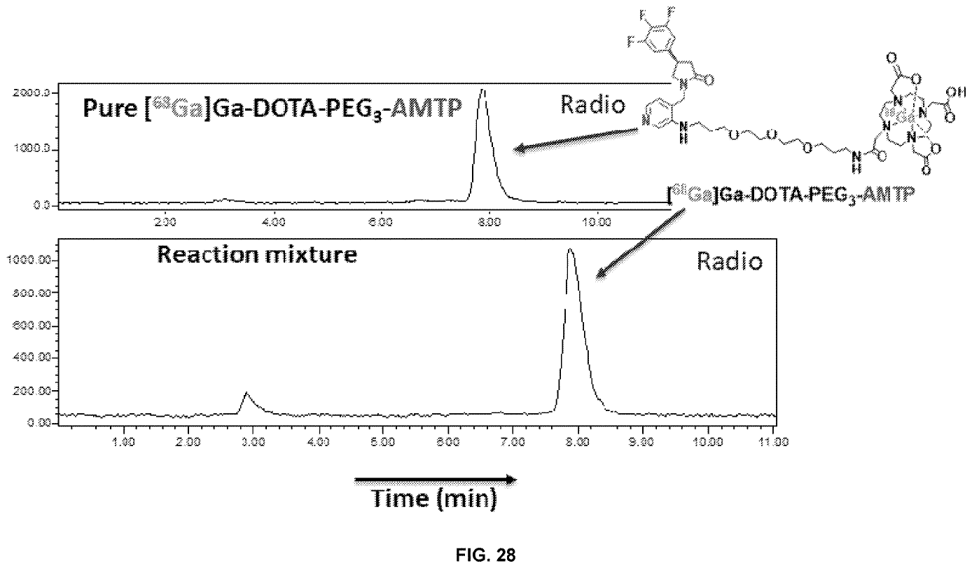

To a 1.5 mL vial containing 5 μg of DOTA-PEG₃-AMTP in 150 μL of 2 M HEPES (pH=7) solution was added 17.3 mCi of ⁶⁸GaCl₃ in 0.1 M HCl. The reaction mixture (pH=4.5) and was incubated at 99° C. for 10 min. 5 μL of 5 mM EDTA was added to the reaction mixture, which was allowed to incubate for another 5 min (EDTA was used to remove non-specifically bound or free ⁶⁸Ga from the reaction mixture). The reaction mixture was diluted to 30 mL with milli-Q water and the purification of [⁶⁸Ga]Ga-DOTA-PEG₃-AMTP was performed by passing the diluted mixture through a Sep-Pak C-18 light cartridge. After rinsing the cartridge two times with 5 mL water, the product [⁶⁸Ga]Ga-DOTA-PEG₃-AMTP was eluted out by pure 1 mL ethanol. The product was analyzed by radio-HPLC to determine the radiochemical purity ((>99%, FIG. 28). 8.50 mCi pure product was obtained at the end of synthesis with molar activity 1660 mCi/μmol and RCY ~90%. The partition coefficient log P of [⁶⁸Ga]Ga-DOTA-PEG₃-AMTP was-0.49 (n=3), determined by the ratio of decay-corrected radioactivity concentrations in n-octanol and PBS. The. In vitro stability of [⁶⁸Ga]Ga-DOTA-PEG₃-AMTP was analyzed with human serum which showed ~12% decomposition at 3.5 h.

The table indicates the comparison of the Examples 1-3.

| PET Conjugates | RCY | RCP | Molar Activity | logP |
|---|---|---|---|---|
| [⁶⁴Cu]Cu-CB-TE2A-(PEG₃- | ~22% | >99% | 467 mCi/μmol | 0.37 |
| AMTP)₂ | | | | |
| [⁶⁴Cu]Cu-CB-TE2A-PEG₃- | ~51% | >94% | 736 mCi/μmol | −0.49 |
| AMTP | | | | |
| [⁶⁸Ga]Ga-DOTA-PEG₃- | ~90% | >99% | 1660 mCi/μmol | −2.07 |
| AMTP | | | | |

### Example 8: PET Imaging of SV2A with [⁶⁸Ga]Ga-DOTA-PEG₃-AMTP in NEPC Xenograft Model

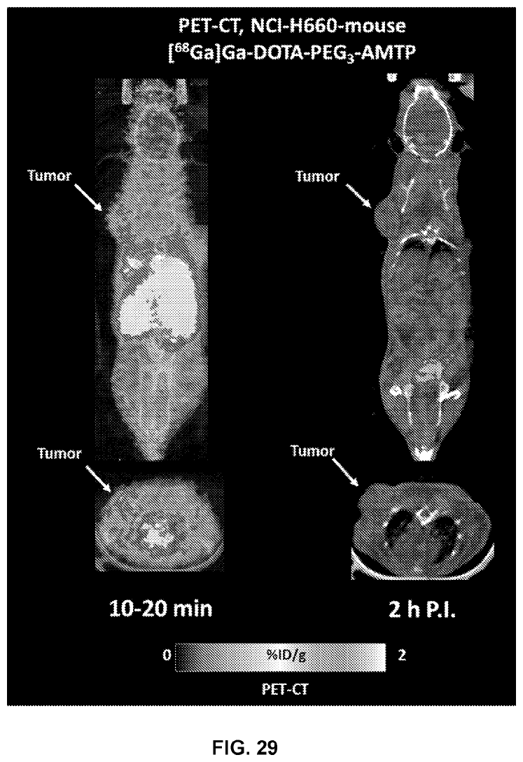

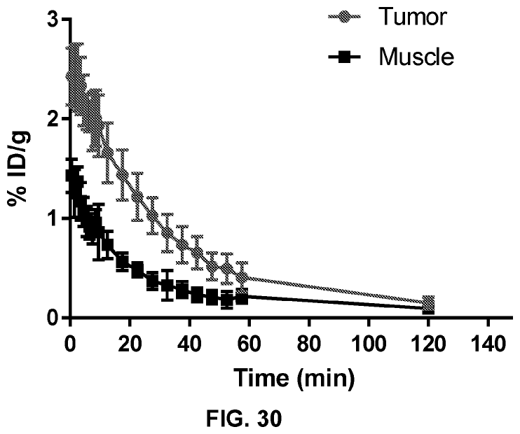

PET/CT imaging of SCID mice bearing SV2A⁺ NCI-H660 (high-SV2A expressing) tumor xenografts were evaluated by 0-60 min real-time dynamic PET scan followed by 7 min CT. An additional 20 min PET scan was performed at 2 h post injection (p.i.) followed by 7 min CT. The PET/CT images showed that SV2A⁺ NCI-H660 tumors were clearly observable (FIG. 29). Quantitative uptake analysis (FIG. 30) showed that [⁶⁸Ga]Ga-DOTA-PEG₃-AMTP has relatively higher tumor uptake than muscle at all-time points throughout the scan. The larger and hydrophilic molecular construction reduces the BBB penetration and showed minimal brain uptake in dynamic time points. Fast tracer clearance from the tumor reduces the potency of [⁶⁸Ga]Ga-DOTA-PEG₃-AMTP for farther pre-clinical evaluation.

### Example 11: IHC Results

The IHC staining of SV2A on the innervated tumors demonstrated the localization of the SV2A in the tumors. The expression SV2A in DU145 tumor is in both cytosol and the cell membrane while it is mostly in the cell membrane in H720 and H727. The optical density of the DAB staining fit the trend of the trace uptake in the tumors.

### Example 12: IHC Method

The paraffined slides were dewaxed and the antigen was retrieved with citrate buffer. Slides were blocked by intelliPATH™ Background Punisher (IP974G20, BioCare Medical, USA), followed by peroxidase inhibitor (IPB5000G20, BioCare Medical, USA) and mouse antigen killer (RBM961G, BioCare Medical, USA). The slides were stained by anti-SV2A antibody (HPA007863, Sigma, USA) for 1 hr at room temperature and stained with a universal secondary antibody conjugated with HRP (M2U522G, BioCare Medical, USA). The SV2A expression was visualized by DAB staining with hematoxylin to stain the cytosol and nucleus.

### REFERENCES

The following references are incorporated by reference in their entireties.
1. Tsang, J. Y.; Tse, G. M., Breast cancer with neuroendocrine differentiation: an update based on the latest WHO classification. Modern Pathology 2021, 34(6), 1062-1073.
2. Duan, K.; Mete, O., Algorithmic approach to neuroendocrine tumors in targeted biopsies: Practical applications of immunohistochemical markers. Cancer Cytopathology 2016, 124 (12), 871-884.
3. Yamada, Y.; Beltran, H., Clinical and Biological Features of Neuroendocrine Prostate Cancer. Current Oncology Reports 2021, 23 (2), 15.
4. Kleist, B.; Poetsch, M., Neuroendocrine differentiation: The mysterious fellow of colorectal cancer. World J Gastroenterol 2015, 21 (41), 11740-7.
5. Survival Rates for Prostate Cancer. American Cancer Society, Inc. Accessed on Aug. 27, 2020 via https://www.cancer.org/cancer/prostate-cancer/detection-diagnosis-staging/survival-rates.html.
6. Beltran, H.; Prandi, D.; Mosquera, J. M.; Benelli, M.; Puca, L.; Cyrta, J.; Marotz, C.; Giannopoulou, E.; Chakravarthi, B. V.; Varambally, S.; Tomlins, S. A.; Nanus, D. M.; Tagawa, S. T.; Van Allen, E. M.; Elemento, O.; Sboner, A.; Garraway, L. A.; Rubin, M. A.; Demichelis, F., Divergent clonal evolution of castration-resistant neuroendocrine prostate cancer. Nat Med 2016, 22 (3), 298-305.
7. Conteduca, V.; Aieta, M.; Amadori, D.; De Giorgi, U., Neuroendocrine differentiation in prostate cancer: current and emerging therapy strategies. Crit Rev Oncol Hematol 2014, 92 (1), 11-24.
8. Grigore, A. D.; Ben-Jacob, E.; Farach-Carson, M. C., Prostate Cancer and Neuroendocrine Differentiation: More Neuronal, Less Endocrine? Frontiers in Oncology 2015, 5, Article 37.
9. Palmgren, J. S.; Karavadia, S. S.; Wakefield, M. R., Unusual and underappreciated: small cell carcinoma of the prostate. Semin Oncol 2007, 34 (1), 22-9.
10. Davies, A. H.; Beltran, H.; Zoubeidi, A., Cellular plasticity and the neuroendocrine phenotype in prostate cancer. Nat Rev Urol 2018, 15 (5), 271-286.
11. Carlson, R. H., The Treatment Challenges of Neuroendocrine Prostate Cancer. Oncology Times 2015, 37 (4), 1, 18-19.
12. Beltran, H.; Rickman, D. S.; Park, K.; Chae, S. S.; Sboner, A.; MacDonald, T. Y.; Wang, Y.; Sheikh, K. L.; Terry, S.; Tagawa, S. T.; Dhir, R.; Nelson, J. B.; de la Taille, A.; Allory, Y.; Gerstein, M. B.; Perner, S.; Pienta, K. J.; Chinnaiyan, A. M.; Wang, Y.; Collins, C. C.; Gleave, M. E.; Demichelis, F.; Nanus, D. M.; Rubin, M. A., Molecular Characterization of Neuroendocrine Prostate Cancer and Identification of New Drug Targets. Cancer Discovery 2011, 1 (6), 487-495.
13. di Sant′Agnese, P. A., Neuroendocrine differentiation in carcinoma of the prostate. Diagnostic, prognostic, and therapeutic implications. Cancer 1992, 70 (1 Suppl), 254-68.
14. Zahalka, A. H.; Frenette, P. S., Nerves in cancer. Nat Rev Cancer 2020, 20 (3), 143-157.
15. Venkatesh, H. S.; Morishita, W.; Geraghty, A. C.; Silverbush, D.; Gillespie, S. M.; Arzt, M.; Tam, L. T.; Espenel, C.; Ponnuswami, A.; Ni, L.; Woo, P. J.; Taylor, K. R.; Agarwal, A.; Regev, A.; Brang, D.; Vogel, H.; Hervey-Jumper, S.; Bergles, D. E.; Suva, M. L.; Malenka, R. C.; Monje, M., Electrical and synaptic integration of glioma into neural circuits. Nature 2019, 573 (7775), 539-545.
16. Venkataramani, V.; Tanev, D. I.; Strahle, C.; Studier-Fischer, A.; Fankhauser, L.; Kessler, T.; Korber, C.; Kardorff, M.; Ratliff, M.; Xie, R.; Horstmann, H.; Messer, M.; Paik, S. P.; Knabbe, J.; Sahm, F.; Kurz, F. T.; Acikgoz, A. A.; Herrmannsdorfer, F.; Agarwal, A.; Bergles, D. E.; Chalmers, A.; Miletic, H.; Turcan, S.; Mawrin, C.; Hanggi, D.; Liu, H. K.; Wick, W.; Winkler, F.; Kuner, T., Glutamatergic synaptic input to glioma cells drives brain tumour progression. Nature 2019, 573 (7775), 532-538.
17. Zeng, Q.; Michael, I. P.; Zhang, P.; Saghafinia, S.; Knott, G.; Jiao, W.; McCabe, B. D.; Galvan, J. A.; Robinson, H. P. C.; Zlobec, I.; Ciriello, G.; Hanahan, D., Synaptic proximity enables NMDAR signalling to promote brain metastasis. Nature 2019, 573 (7775), 526-531.
18. Mauffrey, P.; Tchitchek, N.; Barroca, V.; Bemelmans, A.; Firlej, V.; Allory, Y.; Romeo, P. H.; Magnon, C., Progenitors from the central nervous system drive neurogenesis in cancer. Nature 2019, 569 (7758), 672-678.
19. Magnon, C.; Hall, S. J.; Lin, J.; Xue, X.; Gerber, L.; Freedland, S. J.; Frenette, P. S., Autonomic Nerve Development Contributes to Prostate Cancer Progression. Science 2013, 341 (6142), 1236361.
20. Ayala, G. E.; Dai, H.; Powell, M.; Li, R.; Ding, Y.; Wheeler, T. M.; Shine, D.; Kadmon, D.; Thompson, T.; Miles, B. J.; Ittmann, M. M.; Rowley, D., Cancer-related axonogenesis and neurogenesis in prostate cancer. Clin Cancer Res 2008, 14 (23), 7593-603.
21. Ayala, G. E.; Dai, H.; Ittmann, M.; Li, R.; Powell, M.; Frolov, A.; Wheeler, T. M.; Thompson, T. C.; Rowley, D., Growth and Survival Mechanisms Associated with Perineural Invasion in Prostate Cancer. Cancer Research 2004, 64 (17), 6082-6090.
22. Ayala, G. E.; Wheeler, T. M.; Shine, H. D.; Schmelz, M.; Frolov, A.; Chakraborty, S.; Rowley, D., In vitro dorsal root ganglia and human prostate cell line interaction: redefining perineural invasion in prostate cancer. Prostate 2001, 49 (3), 213-23.
23. Zhu, Y.; Liu, C.; Cui, Y.; Nadiminty, N.; Lou, W.; Gao, A. C., Interleukin-6 induces neuroendocrine differentiation (NED) through suppression of RE-1 silencing transcription factor (REST). The Prostate 2014, 74 (11), 1086-1094.
24. Beltran, H.; Jendrisak, A.; Landers, M.; Mosquera, J. M.; Kossai, M.; Louw, J.; Krupa, R.; Graf, R. P.; Schreiber, N. A.; Nanus, D. M.; Tagawa, S. T.; Marrinucci, D.; Dittamore, R.; Scher, H. I., The Initial Detection and Partial Characterization of Circulating Tumor Cells in Neuroendocrine Prostate Cancer. Clin Cancer Res 2016, 22 (6), 1510-9.
25. Choi, S. Y. C.; Ettinger, S. L.; Lin, D.; Xue, H.; Ci, X.; Nabavi, N.; Bell, R. H.; Mo, F.; Gout, P. W.; Fleshner, N. E.; Gleave, M. E.; Collins, C. C.; Wang, Y., Targeting MCT4 to reduce lactic acid secretion and glycolysis for treatment of neuroendocrine prostate cancer. Cancer Med 2018, 7 (7), 3385-3392.
26. Terry, S.; Beltran, H., The many faces of neuroendocrine differentiation in prostate cancer progression. Front Oncol 2014, 4, Article 60.
27. Parida, G. K.; Tripathy, S.; Datta Gupta, S.; Singhal, A.; Kumar, R.; Bal, C.; Shamim, S. A., Adenocarcinoma Prostate With Neuroendocrine Differentiation: Potential Utility of ¹⁸F-FDG PET/CT and ⁶⁸Ga-DOTANOC PET/CT Over ⁶⁸Ga-PSMA PET/CT. Clinical Nuclear Medicine 2018, 43 (4), 248-249.
28. Schmitt, A. M.; Blank, A.; Marinoni, I.; Komminoth, P.; Perren, A., Histopathology of NET: Current concepts and new developments. Best Pract Res Clin Endocrinol Metab 2016, 30 (1), 33-43.
29. Tomita, T., Significance of chromogranin A and synaptophysin in pancreatic neuroendocrine tumors. Bosn J Basic Med Sci 2020, 20 (3), 336-346.
30. Karsenty, G.; Rocha, J.; Chevalier, S.; Scarlata, E.; Andrieu, C.; Zouanat, F. Z.; Rocchi, P.; Giusiano, S.; Elzayat, E. A.; Corcos, J., Botulinum toxin type A inhibits the growth of LNCaP human prostate cancer cells in vitro and in vivo. Prostate 2009, 69 (11), 1143-50.
31. Bandala, C.; Cortes-Algara, A. L.; Mejia-Barradas, C. M.; Ilizaliturri-Flores, I.; Dominguez-Rubio, R.; Bazan-Mendez, C. I.; Floriano-Sanchez, E.; Luna-Arias, J. P.; Anaya-Ruiz, M.; Lara-Padilla, E., Botulinum neurotoxin type A inhibits synaptic vesicle 2 expression in breast cancer cell lines. Int J Clin Exp Patho 2015, 8 (7), 8411-8418.
32. Jiang, S.-H.; Hu, L.-P.; Wang, X.; Li, J.; Zhang, Z.-G., Neurotransmitters: emerging targets in cancer. Oncogene 2020, 39 (3), 503-515.
33. DeLellis, R. A.; Shin, S. J.; Treaba, D. O., Chapter 10-Immunohistology of Endocrine Tumors. In Diagnostic Immunohistochemistry (Third Edition), Dabbs, D. J., Ed. W.B. Saunders: Philadelphia, 2011; pp 291-339.
34. Bhargava, R.; Dabbs, D. J., Chapter 8-Immunohistology of Metastatic Carcinomas of Unknown Primary. In Diagnostic Immunohistochemistry (Third Edition), Dabbs, D. J., Ed. W.B. Saunders: Philadelphia, 2011; pp 206-255.
35. Jakobsen, A.-M.; Ahlman, H.; Wängberg, B.; Kolby, L.; Bengtsson, M.; Nilsson, O., Expression of synaptic vesicle protein 2 (SV2) in neuroendocrine tumours of the gastrointestinal tract and pancreas. The Journal of Pathology 2002, 196 (1), 44-50.
36. Georgantzi, K.; Tsolakis, A. V.; Jakobson, A.; Christofferson, R.; Janson, E. T.; Grimelius, L., Synaptic Vesicle Protein 2 and Vesicular Monoamine Transporter 1 and 2 Are Expressed in Neuroblastoma. Endocr Pathol 2019, 30 (3), 173-179.
37. Lu, R.; Fan, C.; Shangguan, W.; Liu, Y.; Li, Y.; Shang, Y.; Yin, D.; Zhang, S.; Huang, Q.; Li, X.; Meng, W.; Xu, H.; Zhou, Z.; Hu, J.; Li, W.; Liu, L.; Mo, X., Neurons generated from carcinoma stem cells support cancer progression. Signal Transduction and Targeted Therapy 2017, 2 (1), e16036.
38. Cortes-Algara, A.; Cardenas-Rodriguez, N.; Lara-Padilla, E.; Floriano-Sanchez, E.; Martinez-Contreras, R.; Anaya-Ruiz, M.; Uribe-Escamilla, R.; Alfaro-Rodriguez, A.; Ilizaliturri-Flores, I.; Perez-Santos, M.; Bandala, C., Synaptic vesicle protein isoforms (SV2A, SV2B, SV2C): expression in breast cancer and their association with risk factors and metastasis in Mexican women. Int J Clin Exp Patho 2017, 10 (2), 1998-2004.
39. Jaskula-Sztul, R.; Whitt, J. D.; Ou, J.; Liu, M. X.; Aburjania, Z.; Chen, H., Synaptic vesicle 2 receptors as a novel targets for neuroendocrine cancer therapy. Journal of Clinical and Translational Science 2018, 2 (S1), 28-29.
40. Stridsberg, M., The Use of Chromogranin, Synaptophysin and Islet Amyloid Polypeptide as Markers for Neuroendocrine Tumours. Upsala Journal of Medical Sciences 1995, 100 (3), 169-199.
41. Estrada, S.; Lubberink, M.; Thibblin, A.; Sprycha, M.; Buchanan, T.; Mestdagh, N.; Kenda, B.; Mercier, J.; Provins, L.; Gillard, M.; Tytgat, D.; Antoni, G., [¹¹C]UCB-A, a novel PET tracer for synaptic vesicle protein 2A. Nuclear Medicine and Biology 2016, 43 (6), 325-332.
42. Nabulsi, N. B.; Mercier, J.; Holden, D.; Carré, S.; Najafzadeh, S.; Vandergeten, M. C.; Lin, S. F.; Deo, A.; Price, N.; Wood, M.; Lara-Jaime, T.; Montel, F.; Laruelle, M.; Carson, R. E.; Hannestad, J.; Huang, Y., Synthesis and Preclinical Evaluation of ¹¹C-UCB-J as a PET Tracer for Imaging the Synaptic Vesicle Glycoprotein 2A in the Brain. J Nucl Med 2016, 57 (5), 777-84.
43. Becker, G.; Warnier, C.; Serrano, M. E.; Bahri, M. A.; Mercier, J.; Lemaire, C.; Salmon, E.; Luxen, A.; Plenevaux, A., Pharmacokinetic Characterization of [¹⁸F]UCB-H PET Radiopharmaceutical in the Rat Brain. Molecular Pharmaceutics 2017, 14 (8), 2719-2725.
44 Li, S.; Cai, Z.; Wu, X.; Holden, D.; Pracitto, R.; Kapinos, M.; Gao, H.; Labaree, D.; Nabulsi, N.; Carson, R. E.; Huang, Y., Synthesis and in Vivo Evaluation of a Novel PET Radiotracer for Imaging of Synaptic Vesicle Glycoprotein 2A (SV2A) in Nonhuman Primates. ACS Chem Neurosci 2019, 10 (3), 1544-1554.
45. Cai, Z.; Li, S.; Zhang, W.; Pracitto, R.; Wu, X.; Baum, E.; Finnema, S. J.; Holden, D.; Toyonaga, T.; Lin, S.-f.; Lindemann, M.; Shirali, A.; Labaree, D. C.; Ropchan, J.; Nabulsi, N.; Carson, R. E.; Huang, Y., Synthesis and Preclinical Evaluation of an ¹⁸F-Labeled Synaptic Vesicle Glycoprotein 2A PET Imaging Probe: [¹⁸F]SynVesT-2. ACS Chemical Neuroscience 2020, 11 (4), 592-603.
46. Cai, Z. X.; Li, S. Y.; Matuskey, D.; Nabulsi, N.; Huang, Y. Y., PET imaging of synaptic density: A new tool for investigation of neuropsychiatric diseases. Neurosci Lett 2019, 691, 44-50.
47. Becker, G.; Dammicco, S.; Bahri, M. A.; Salmon, E., The Rise of Synaptic Density PET Imaging. Molecules 2020, 25 (10), e2303.
48. Guan, B.; Zhou, N.; Wu, C.-Y.; Li, S.; Chen, Y.-A.; Debnath, S.; Hofstad, M.; Ma, S.; Raj, G. V.; He, D., Validation of SV2A-Targeted PET Imaging for Noninvasive Assessment of Neuroendocrine Differentiation in Prostate Cancer. International journal of molecular sciences 2021, 22 (23), 13085.
49. Kumar, A.; Mastren, T.; Wang, B.; Hsieh, J.-T.; Hao, G.; Sun, X., Design of a small-molecule drug conjugate for prostate cancer targeted theranostics. Bioconjugate chemistry 2016, 27 (7), 1681-1689.
50. Li, S.; Cai, Z.; Zhang, W.; Holden, D.; Lin, S.-f.; Finnema, S. J.; Shirali, A.; Ropchan, J.; Carre, S.; Mercier, J., Synthesis and in vivo evaluation of [18 F]UCB-J for PET imaging of synaptic vesicle glycoprotein 2A (SV2A). European journal of nuclear medicine and molecular imaging 2019, 46 (9), 1952-1965.
51. Hao, G.; Kumar, A.; Dobin, T.; Öz, O. K.; Hsieh, J.-T.; Sun, X., A multivalent approach of imaging probe design to overcome an endogenous anion binding competition for noninvasive assessment of prostate specific membrane antigen. Molecular pharmaceutics 2013, 10 (8), 2975-2985.

What is claimed is:

1. A compound of Formula (I): 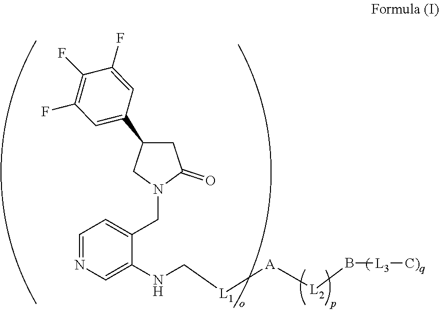
wherein L₁, L₂, and L₃ are linkers and are independently selected from a group consisting of at least one peptide linker, at least one polyethylene glycol (PEG) linker, a disulfide linker, an albumin binding entity, absent, and a combination thereof,
A is a metal chelator or a chemotherapeutic agent,
B is a heterocyclic ring, a polyamidoamine dendrimer, a peptide, a disulfide, or absent,
C is a chemotherapeutic agent, 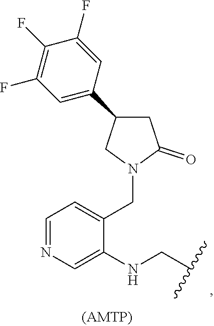 [SMILES: *CNc1cnccc1CN1C[C@@H](c2cc(F)c(F)c(F)c2)CC1=O]
or absent,
o is an integer from 0 to 6,
p is an integer from 0 or 1,
q is an integer from 1 to 6, and
wherein when o is 0, C is AMTP.

2. The compound of claim 1, wherein the polyethylene glycol (PEG) linker comprises from 1 to 12 ethylene glycol repeating units, an albumin binding entity, or a combination thereof.

3. The compound of either claim 1 or 2, wherein the albumin binding entity comprises a fragment consisting of an azo dye Evans Blue fragment, a 4-(p-iodophenyl)butyryl fragment, a naphthalene acyl sulfonamide fragment, a diphenylcyclohexanol phosphate ester fragment, a 9-fluorenylmethooxycarbonyl fragment, a Fmoc derivative linked to a 16-sulfanylhexadecanoic acid, a dicoumarol fragment, a divalent diflunisal-indomethacin moiety linked through a γGlu-Lys dipeptide coupled to a unit of 8-amino-3,6-dioxaoctanoic acid (O2Oc) fragment, a lithocholic acid coupled to a γGlu linker fragment, a lithocholic acid coupled to a γGlu fragment, a 6-(4-(4-iodophenyl)butanamido)hexanoate coupled to carboxyfluorescein through a d-Lys fragment, a A083/B134 fragment, a A099/B344 fragment, a linear peptide 89D03: Ac-WWEQDRDWDFDVFGGGTP-NH₂ fragment, an acylated heptapeptide F-tag: fluorescein-EYEK (palmitate) EYE-NH₂ fragment, a disulfide cyclized peptide SA21, Ac-RLIEDICLPRWGCLWEDD-NH₂ fragment, a head-to-tail cyclized peptide HSA-1: AK*K*PGK*AK*PG with variable lysine (K*) fragment, a bacterial ABD scaffold, human neonatal Fc receptor (FcRn), a bacterial protein Sso7d, a DARPin protein domain, a single-domain antibody, a fab domain, a nanobody, or a VNAR domain.

4. The compound of any one of claims 1-3, wherein the heterocyclic ring is selected from a group consisting of a triazine, a pyrimidine, a piperazine, a pyrazine, an imidazole, a pyrazole, and a pyridine.

5. The compound of claim 4, wherein the heterocyclic ring is a triazine.

6. The compound of any one of claims 1-5, wherein the polyamidoamine dendrimer (PAMAM dendrimer) is a generation 0 (G0) polyamidoamine dendrimer, a generation 1 (G1) polyamidoamine dendrimer, or a generation 2 (G2) polyamidoamine dendrimer.

7. The compound of any one of claims 1-6, wherein the peptide linker comprises 2 to about 10 amino acid residues, an albumin binding entity, or a combination thereof.

8. The compound of claim 7, wherein the peptide linker comprises a lysine residue.

9. The compound of any one of claims 1-8, wherein the chemotherapeutic agent is a highly potent chemodrug molecule.

10. The compound of claim 9, wherein the chemodrug molecule is an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

11. The compound of any one of claims 1-10, wherein the metal chelator is selected from a group consisting of 1,4,7-triazacyclononane-1,4,7-triacetic acid (NOTA), 5-(8-methyl-3,6, 10, 13, 16, 19-hexaaza-bicyclo[6.6.6]icosan-1-ylamino)-5-oxopentanoic acid (MeCOSar), 1,4,7,10-tetraazacyclododecane-1,4,7,10-tetraacetic acid (DOTA), 4,11-bis(carboxymethyl)-1,4,8,11tetraazabicyclo[6.6.2]hexadecane (CB-TE2A), and N,N′-bis-[2-hydroxy-5-(carboxyethyl)benzyl]ethylenediamine-N,N′-diacetic acid (HBED-CC).

12. The compound of any one of claims 1-11, wherein the metal chelator is incorporated to label the compounds with one metal radionuclide (M).

13. The compound of claim 12, wherein the radionuclide comprises ⁶⁷Ga, ¹¹¹In, ^{99m}Tc, ¹³¹I, ¹²³I, ¹²⁵I, ¹⁰³Pd, ⁶⁸Ga, ¹⁸F, ⁶⁰Cu, ⁶¹Cu, ⁶²Cu, ⁶⁴Cu, ⁹⁰Y, ⁶⁷Cu, ¹⁸⁶Re, ¹⁸⁸Re, ¹⁹⁸Au, ¹⁵³Sm, ¹⁷⁷Lu, ²¹³Bi, ²¹²Pb, ²²³Ra, ²¹¹At, or ²²⁵Ac.

14. The compound of any one of claims 1-13, wherein at least one fluorine of the compound of Formula (I) comprises ¹⁸F.

15. The compound of any one of claims 1-14, wherein the compound of Formula (I) is targeted to Synaptic Vesicle Glycoprotein 2A (SV2A).

16. The compound of any one of claims 1-15, wherein the compound of Formula (I) does not cross the blood-brain barrier.

17. The compound of any one of claims 1-16, wherein the compound of Formula (I) maintains >90% intact in human serum at 37° C. for at least 4 hours.

18. The compound of any one of claims 1-17, wherein the A is NOTA; B is absent; C is absent; L₁ is a linker comprising from 1 to 12 ethylene glycol repeating units; L₂ and L₃ are absent; o is 3; p is 0; and q is 0; as shown in the compound of Formula (II):

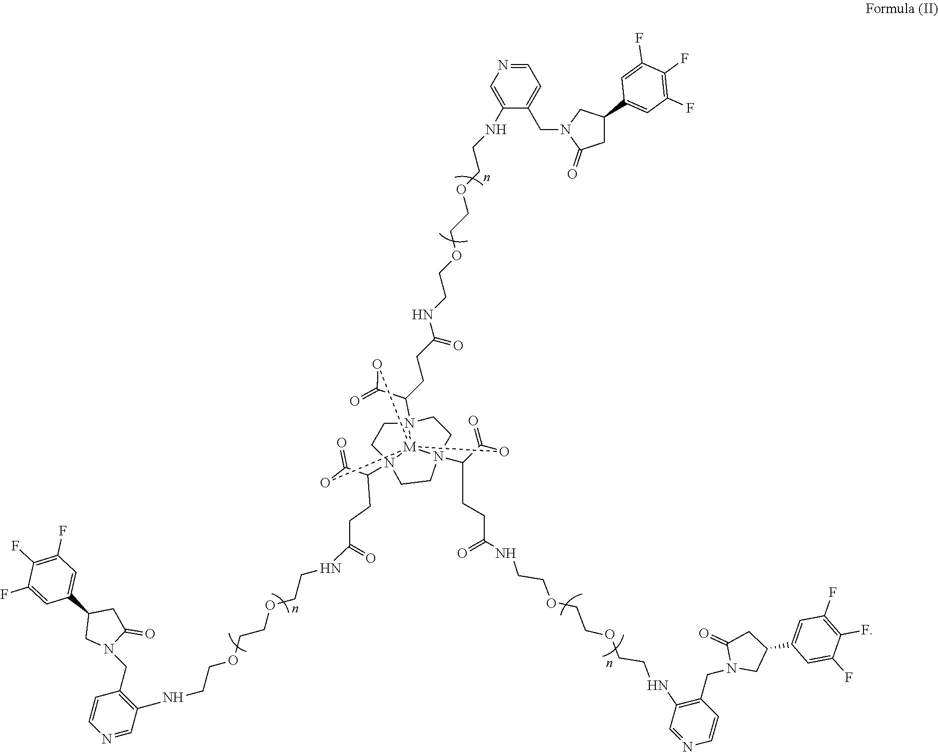

19. The compound of any one claims 1-17, wherein A is a metal chelator selected from the group consisting of MeCOSar, DOTA, CB-TE2A, and NOTA; B is a peptide; C is AMTP; L₁ is absent; L₂ is a peptide linker; L₃ is a linker comprising from 1 to 12 ethylene glycol repeating units; o is 0; p is 1; and m is 2; as shown in the compound of Formula (III):

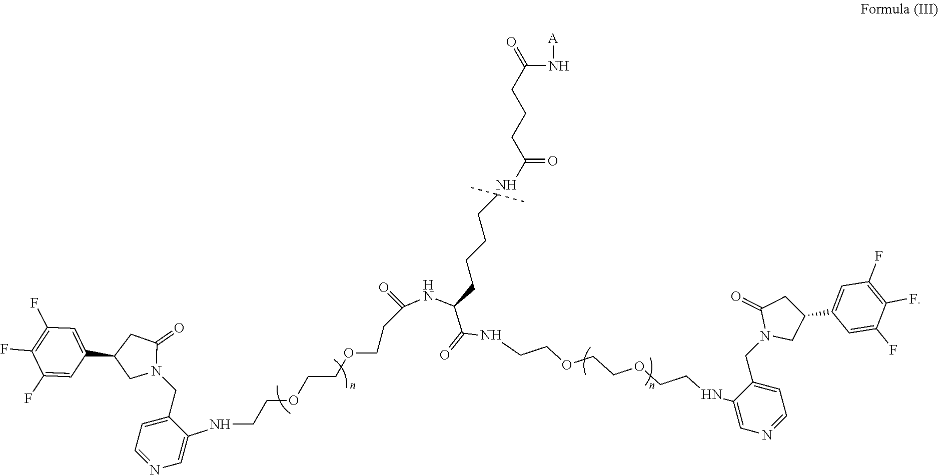

20. The compound of any one claims 1-17, wherein A is a metal chelator selected from the group consisting of MeCOSar, DOTA, CB-TE2A, and NOTA; B is triazine; C is AMTP; L₁ is absent; L₂ and L₃ are linkers comprising from 1 to 12 ethylene glycol repeating units; o is 0; p is 1; and q is 2; as shown in the compound of Formula (IV):

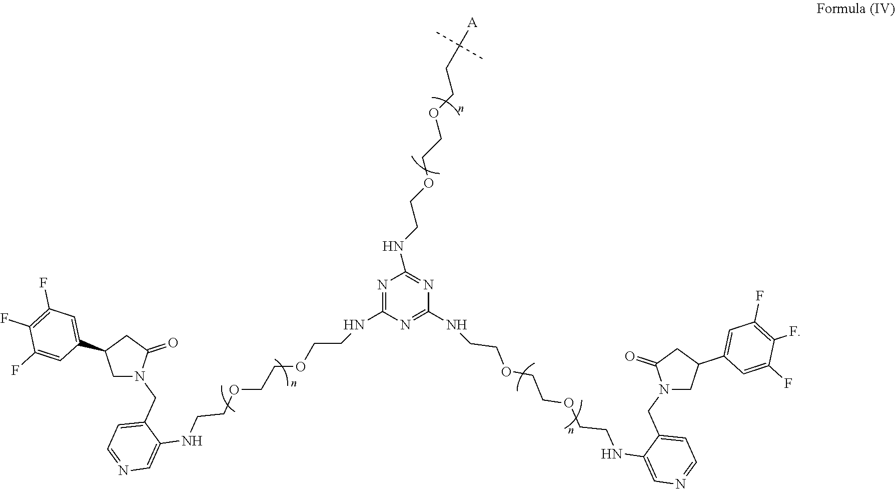

21. The compound of any one claims 1-17, wherein A is a metal chelator selected from the group consisting of MeCOSar, DOTA, CB-TE2A, and NOTA; B is a peptide; C is AMTP; L₁ is absent; L₂ is a peptide linker and an albumin binding entity; L₃ is a peptide linker; o is 0; p is 1; and q is an integer from 1 to 6; as shown in the compound of Formula (V):

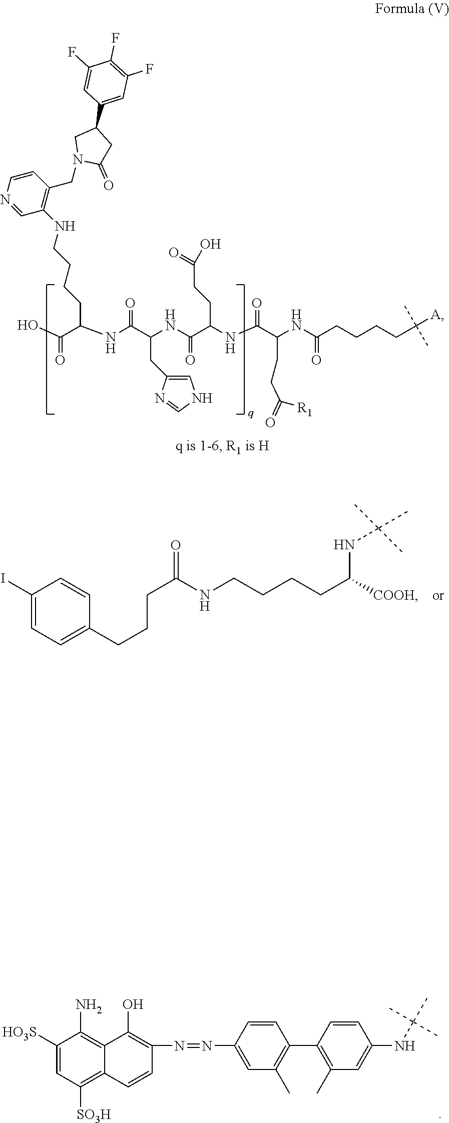

22. The compound of any one claims 1-17, wherein A is a metal chelator selected from the group consisting of MeCOSar, DOTA, CB-TE2A, and NOTA; B is a peptide; C is AMTP; L₁ is absent; L₂ is a peptide linker and an albumin binding entity; L₃ is a peptide linker; o is 0; p is 1; and q is an integer from 1 to 6; as shown in the compound of Formula (VI):

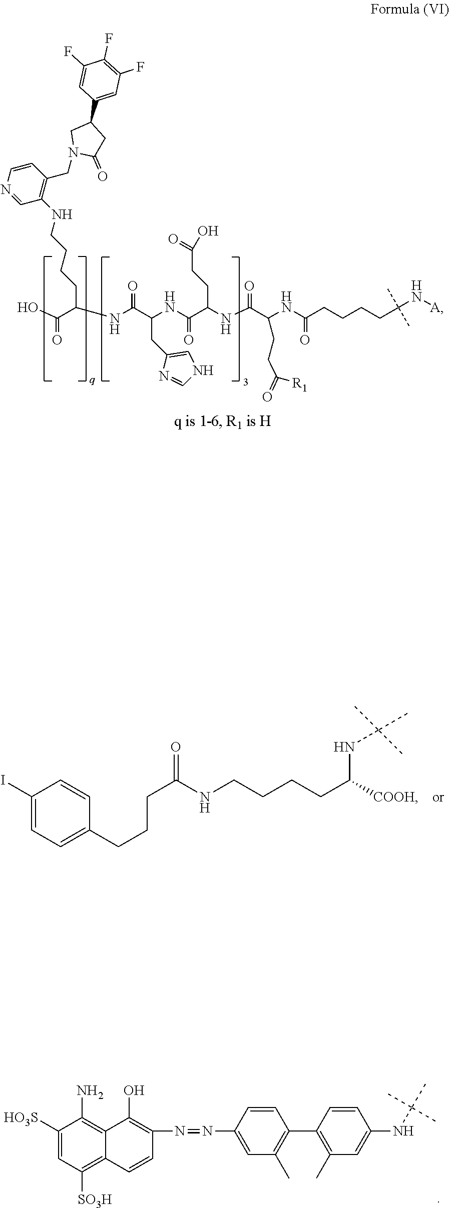

23. The compound of any one claims 1-17, wherein A is a metal chelator selected from the group consisting of MeCOSar, DOTA, CB-TE2A, and NOTA; B is absent; C is absent; L₁ is an albumin binding entity and an ethylene glycol linker comprising 3 ethylene glycol repeating units; L₂ and L₃ are absent; o is 1; p is 0; and q is 0; as shown in the compound of Formula (VII):

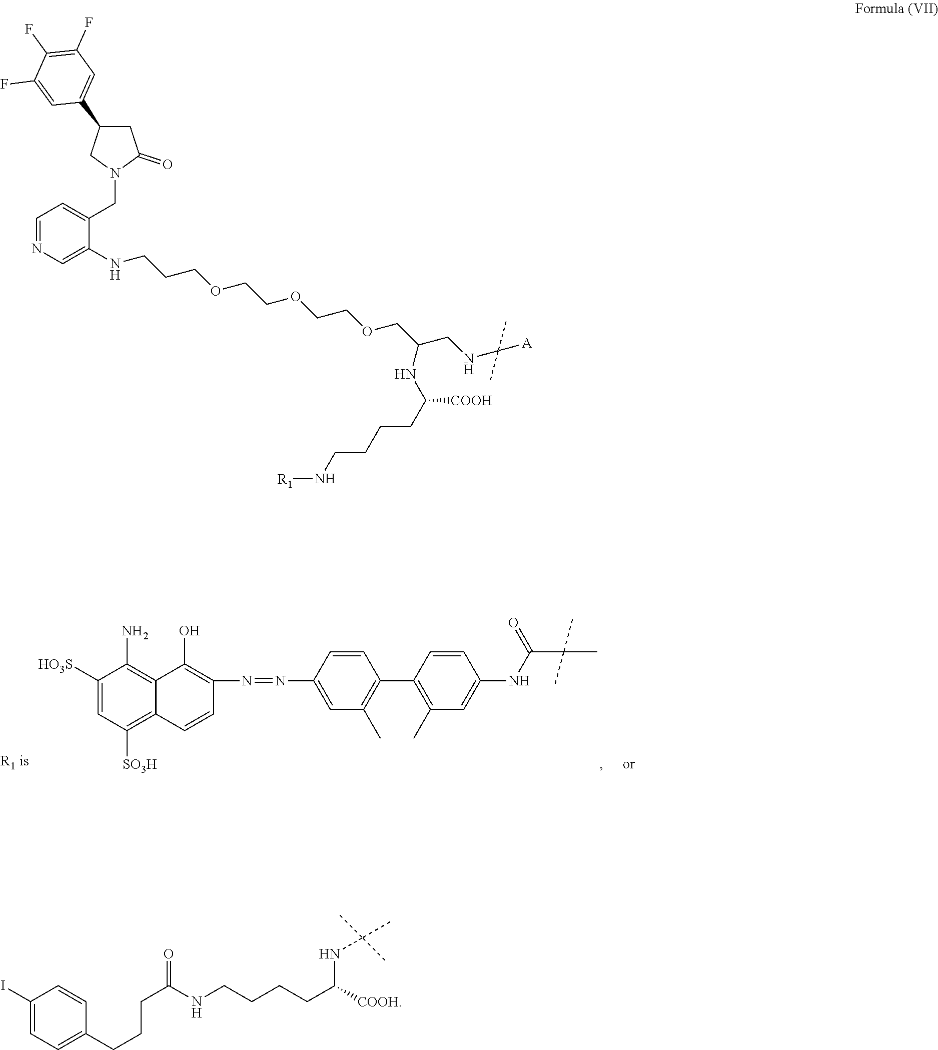

24. The compound of any one of claims 1-17, wherein the A is NOTA; B is triazine; C is AMTP; and L₁, L₂, and L₃ are linkers comprising 3 ethylene glycol repeating units; o is 2; p is 1; and q is 2; as shown in the compound of Formula (VIII):

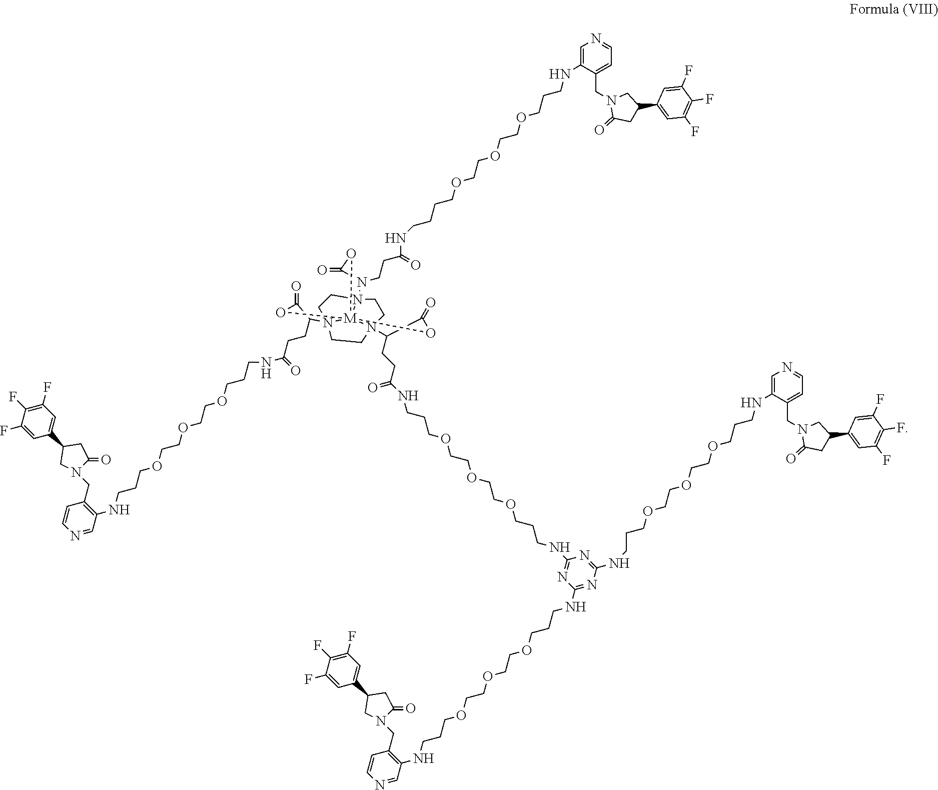

25. The compound of any one of claims 1-17, wherein the A is HBED-CC; B is absent; C is absent; L₁ is a linker comprising 3 ethylene glycol repeating unit and a peptide; L₂ and L₃ are absent; and o is 1; p is 0; and q is 0; as shown in the compound of Formula (IX):

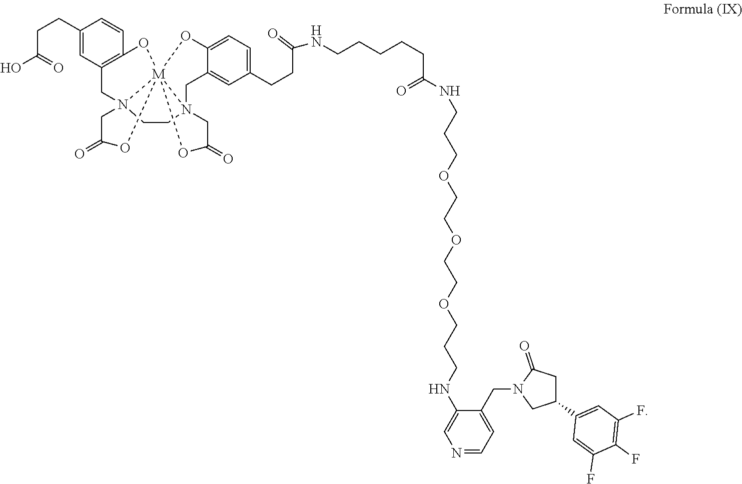

26. The compound of any one of claims 1-17, wherein the A is HBED-CC; B is absent; C is absent; L₁ is a linker comprising 3 ethylene glycol repeating units and a peptide; L₂ and L₃ are absent; o is 2; p is 0; and q is 0; as shown in the compound of Formula (X):

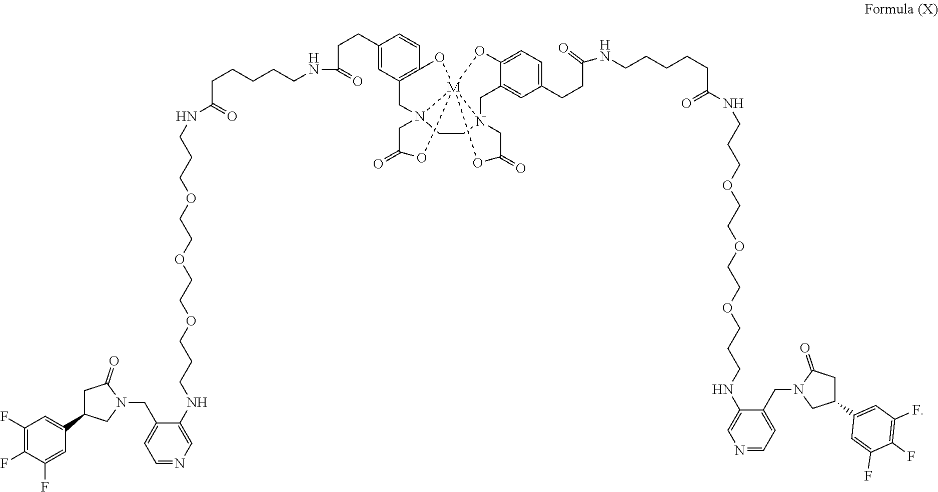

27. The compound of any one of claims 1-17, wherein the A is DM1; B is absent; C is absent; L₁ is a linker comprising 3 ethylene glycol repeating units and a disulfide group; L₂ and L₃ are absent; o is 1; p is 0; and q is 0; as shown in the compound of Formula (XI):

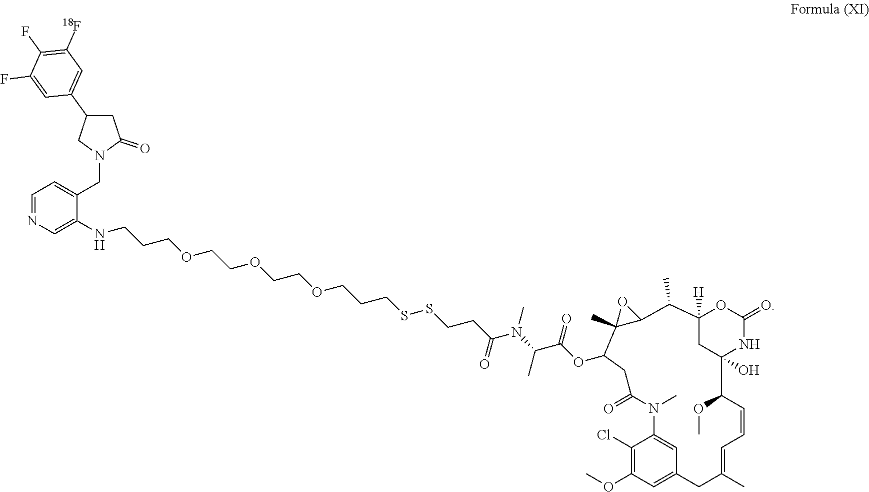

28. The compound of any one of claims 1-17, wherein the A is FTY720; B is absent; C is absent; L₁ is a linker comprising 3 ethylene glycol repeating unit and a disulfide group; L₂ and L₃ are absent; o is 1; p is 0; and q is 0; as shown in the compound of Formula (XII):

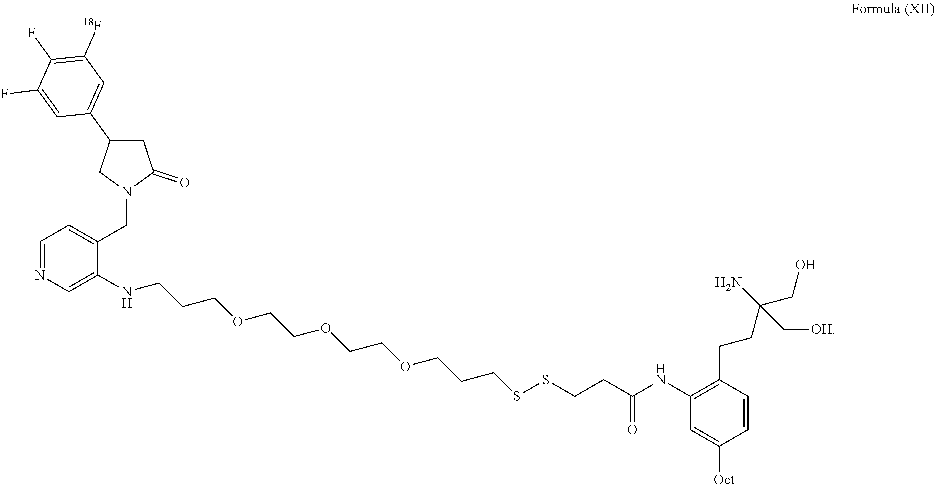

29. The compound of any one of claims 1-17, wherein the A is NOTA; B is a peptide; C is DM1 or FTY720; L₁ is a linker comprising from 1 to 12 ethylene glycol repeating units; L₂ is a linker comprising from 1 to 12 ethylene glycol repeating units; L₃ is a disulfide linker; o is 2; p is 1; and q is 1; as shown in the compound of Formula (XIII):

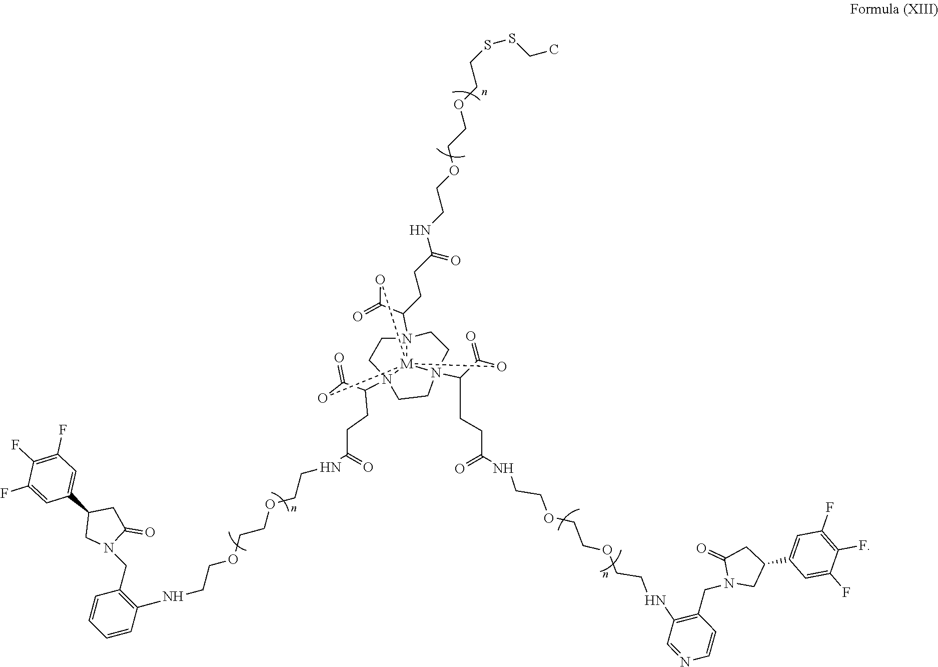

30. The compound of any one of claims 1-17, wherein the A is DM1 or FTY720; B is triazine; C is [¹⁸F]AMTP; L₁ is absent; L₂ is a linker comprising from 1 to 12 ethylene glycol repeating units and a disulfide group; L₃ is a linker comprising from 1 to 12 ethylene glycol repeating units; o is 2; p is 1; and q is 2; as shown in the compound of Formula (XIV):

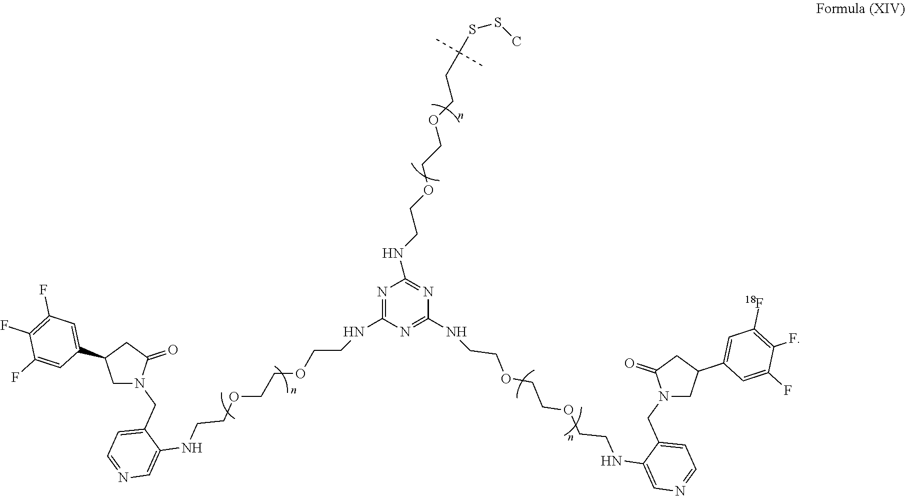

31. The compound of any one of claims 1-17, wherein the A is DM1 or FTY720; B is a peptide; C is AMTP; L₁ is absent; L₂ is a disulfide group and a peptide linker or a peptide linker, an albumin binding entity, and a disulfide group; L₃ is a peptide linker; o is 0; p is 1; q is an integer from 1 to 6; as shown in the compound of Formula (XV):

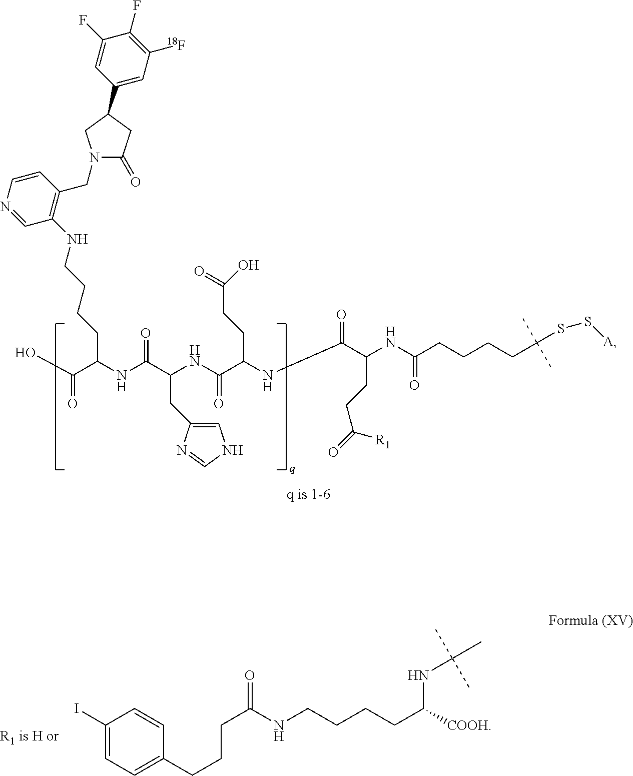

32. A pharmaceutical composition comprising a compound of any one of claims 1-31 and at least one pharmaceutically acceptable excipient or carrier.

33. A method of treating and/or detecting a cancer, the method comprising administering an effective amount of a compound according to any one of claims 1-31 or a pharmaceutical composition according to claim 32 to a subject in need thereof, wherein the subject in need thereof has or is suspected of one or more cancers.

34. The method of claim 33, wherein the one or more cancers comprises one or more innervated cancers, one or more cancers with neuroendocrine differentiation, or any combination thereof.

35. The method of claim 34, wherein the one or more innervated cancers or one or more primary metastases comprises breast cancers, cervical cancers, colon cancer, gastric cancers, gliomas, head-and-neck cancers, melanomas, ovarian cancers, pancreatic cancers, prostate cancers, thyroid cancers, or any combination thereof.

36. The method of claim 33, wherein the one or more cancers or one or more primary metastases with neuroendocrine differentiation comprises small-cell carcinomas, neoplasms, carcinoid, neuroendocrine carcinoma, large cell neuroendocrine carcinomas, prostate cancers, or any combination thereof.

37. The method of any one of claims 33-36, wherein the administering is intravenous or other suitable mode.

38. A method of imaging at least one region of a subject's body, the method comprising:
administering to the subject at least one compound according to any one of claims 1-31 or a pharmaceutical composition according to claim 32; and
detecting the at least one compound by nuclear medicine imaging in at least one region of the body of the subject;
thereby generating an image of the at least one region of the body of the subject.

39. The method of claim 38, wherein the nuclear medicine imaging comprises Single Photon Emission Computed Tomography (SPECT), Positron Emission Tomography (PET), or a combination thereof.

40. A method of monitoring and/or evaluating effectiveness of treatment or therapy for a cancer in a subject's body, the method comprising:
(a) administering to the subject at least one compound according to any one of claims 1-31 or a pharmaceutical composition according to claim 32;
(b) detecting the at least one compound by PET or SPECT, or a combination thereof in at least one region of the body of the subject; and
(c) determining a level of SV2A in the at least one region of the body of the subject, and comparing it to a control reference from the subject before receiving the therapy or treatment,
wherein, if the level of SV2A in the at least one region of the body of the subject is lower than in the control reference, the treatment or therapy is at least partially effective for the subject.

41. The method of claim 40, wherein the cancer comprises one or more innervated cancers, one or more cancers with neuroendocrine differentiation, primary and distal metastases, or any combination thereof.

42. The method of either claim 40 or 41, wherein the treatment or therapy for a cancer and/or primary and distal metastases comprises administration of one or more somatostatin analogs, chemotherapy, targeted therapy, immunotherapy, peptide receptor radionuclide therapy (PRRT), radiotherapy, or any combination thereof.
